(19) United States
(12) Patent Application Publication
(10) Pub. No.: US 2023/0022596 A1
(43) Pub. Date: Jan. 26, 2023

(54) NEOANTIGEN-ELICITING ANTIBODY DRUG CONJUGATES FOR THE IMMUNOTHERAPY OF CANCER

(71) Applicants: CLARENCE HURT, Los Altos, CA (US); Luis Soares, Ormond Beach, FL (US)

(72) Inventors: CLARENCE HURT, Los Altos, CA (US); Luis Soares, Ormond Beach, FL (US)

(21) Appl. No.: 17/846,002

(22) Filed: Jun. 21, 2022

Related U.S. Application Data
(60) Provisional application No. 63/212,562, filed on Jun. 18, 2021.

(51) Int. Cl. A61K 47/68

(52) U.S. Cl. CPC A61K 47/6889

(57) ABSTRACT

Novel neoantigen-eliciting antibody drug conjugates are disclosed. These compounds or pharmaceutically acceptable salts, hydrates, solvates, isomers, or tautomers thereof are useful for the treatment of disorders, including but not limited to pancreatic cancer and other check point positive cancers. More particularly, these compounds may comprise biologically active polypeptides or hormones modified to include the attachment of therapeutic compounds using linkers. The compounds of the disclosure, or pharmaceutically acceptable salts, hydrates, solvates, isomers, or tautomers thereof, also comprise therapeutic compounds connected to linkers.

### CROSS REFERENCE TO RELATED APPLICATIONS

This Non-Provisional patent application claims priority to the co-pending United States patent provisional application having the Ser. No. 63/212,562, filed Jun. 18, 2021, which is incorporated in its entirety by reference.

### TECHNICAL FIELD

The present disclosure relates generally to chemical compounds. More particularly, embodiments relate to pharmaceuticals. Even more particularly, embodiments relate to neoantigen-eliciting antibody drug conjugates.

### BACKGROUND

The fundamental immunological difference between individuals lies in the molecular typing of proteins known by the acronym HLA (from the English Human Leukocyte Antigen) or more universally, MHC (Major Histocompatibility Complex). MHC are proteins expressed on the surface of leukocytes known as antigen presenting cells (APC for Antigen Presenting Cell) that interact directly with another class of proteins expressed on the surface of T-type lymphocytes (CD4 and CD8 T cells) known by the acronym TCR (T cell receiver). However, such an interaction is conditional on a previous event in which a protein fragment derived from any intracellular protein and generated by proteolysis associates with a recognition site on the MHC molecule.

This combination MHC+protein fragment, but none of them in isolation, is then recognized by the TCR. Both MHC and TCR are encoded by highly polymorphic genes and each individual carries 2 alleles of 7 different types of MHC. The genes that code for the TCR (alpha and beta genes) are capable of undergoing somatic rearrangement of blocks of DNA providing the encoding of thousands of variants. However, certain structural characteristics must be maintained to ensure the ability to interact with MHCs. As each individual carries only one genetic code, all this diversity is limited by the coding sequence of each one, and there are no two identical individuals in this respect with the exception of identical twins. This is the only reason why organ or tissue transplantation between individuals is rejected. The protein fragments that are generated by proteolysis and bind to MHC are mostly generated from proteins normally expressed by the individual during his life. This repertoire is, therefore, fixed, and changes only when the organism is invaded by viruses, bacteria or other infectious agents that now living inside the host, produce their own proteins, necessary for their metabolism and reproduction, which in turn will undergo proteolysis generating protein fragments that will bind to the MHC. In some cases, protein fragments generated from ingested or inhaled proteins also bind to MHC and are recognized by the TCR (allergic phenomenon).

This recognition of the MHC+protein fragment by the TCR causes the activation of the CD4 and CD8 T cells that start to reproduce in large numbers and to secrete several products and this process is known as “immune response”. These products produced by T cells act on the cells that host the infectious agents causing the destruction of these agents. Therefore, activation of the T cell from the engagement of the TCR by the MHC+fragment is the triggering element of the immune response. However, these protein fragments generated by proteolysis occur through rules that are also genetically determined because the proteolytic enzymes responsible are fixed and obey specific biochemical rules. Thus, only a few protein fragments are generated from the proteolysis of a particular protein species and of these only one or two fragments have chemical and biophysical characteristics that allow their association with MHC and, therefore, recognition by the TCR.

Evolutionarily, a mechanism has been developed that avoids the constant activation of T cells by this repertoire of fragments generated from the proteolysis of the individual's own proteins. Without this mechanism, an immune response against the individual would cause destruction of the individual's tissues and death. This mechanism is based on the elimination, during the perinatal period, of all T cells that carry a TCR capable of recognizing the MHC+SELF protein fragment, i.e., protein fragment of endogenous origin. This happens during the maturation of T cells originating from precursors from the bone marrow during their passage through the small organ known as the thymus. The thymus has a specific structure and function adapted to the ability to express, on some scale, all the proteins encoded by the individual's genome and to generate the protein fragments that bind to the MHC.

Any T cells that have TCR that recognize MHC+SELF protein fragment at this stage are permanently eliminated from the body. Given the nature of the biochemical rules that allow protein fragments to bind to MHC and the relatively small number of MHC variants (7 genes) responsible for binding about an estimated 1014 distinct protein fragments, certain characteristics of this universe of fragments inevitably become similar, for example as the need for the third amino acid in the fragment to have a hydrophobic characteristic and the eighth amino acid to have a positive electrical characteristic. Statistically, thousands of these fragments will have such characteristics, including fragments derived from viruses or bacteria. In order to avoid the elimination of cells from the system due to the recognition of these common characteristics, a ranking system was established, where only T cells containing TCR recognizing MHC+fragment with high affinity or high avidity are eliminated. Calculations show that the affinity required to induce the death of self-reactive T cells is variable and analogic, and therefore some cells with self-reactive potential will be preserved and others with anti-virus (and other organisms) will be eliminated.

This evolutionary solution that at the same time allows the retention of possibly essential cells, the immune defense against viruses inadvertently allows the development of responses against the individual, thus potentially causing autoimmune diseases. To avoid the development of such autoimmune diseases, nature has developed a set of mechanisms, known as \hat{ } checkpoint inhibition \hat{ } that prevent residual T cells with self-reactive TCR from being activated. Checkpoint inhibition is achieved through a series of ligand-receptor pairs in which a ligand, typically present in an antigen presenting cell or a cancer cell binds to a receptor present in the surface of a T cell or a B cell. Such binding results in a negative signal that cancels out other positive signals coming through other receptors in the T or B cells. Consequently, activation of these immune cells is inhibited. It is, for example, a common mechanism coopted by cancer cells as a mechanism of immune evasion.

The problem with cancer: Cancer results from the genetic transformation of a normal cell into a cell that no longer respects organismal instructions for reproduction and functioning and starts to operate autonomously. This genetic transformation results from mutations normally acquired through carcinogenic agents such as ultraviolet or ionizing radiation, heavy metals and other environmental and chemical toxins. This alteration manifested by the permanent alteration of the nucleotide sequence that makes up the genome is generally of a random nature, but it is preferentially manifested in genes that exercise constant activity, such as those controlling cell proliferation and metabolic circuits, common to all cells of the organism.

These mutations, when they occur in gene coding-regions, generally cause one of two possible changes: (a) the mutation causes an interruption in the reading frame of the gene causing the expression of a truncated or non-functional protein or without a specific activity. (b) the mutation causes the expression of a protein with increased or unregulated activity. Unfortunately, such point mutations in some proteins (initially seven to ten) in a universe of almost 100,000 encoded proteins generally do not happen at positions in the protein that coincidentally are part of a protein fragment generated by proteolysis capable of binding to MHC or if they do, not in residues of importance like those that need to be electrically positively charged, or hydrophobic or bulky, for example. In other words, almost all new mutations are not immunologically relevant. It is estimated that only 1 in 100 mutations has immunogenic potential. Therefore, most cancers are not initially immunogenic and are ignored by the immune system, which causes underperformance of the therapeutic modality known as immunotherapy, especially anti-checkpoint therapy. However, it has been observed over the years that older tumors and, therefore, accumulating more mutations, tend to overexpress checkpoint inhibitor proteins in a likely selectable attempt to inhibit any immune responses against new mutations that may have an immunogenic character. From this fact, the idea of using reagents capable of blocking the action of these checkpoint inhibitors arose.

Prior art is exemplified by antibodies to tumor targets such as HER2 or CD30 alone, with the intent of causing antibody-dependent cell lysis or in ADC form where the same antibodies serve as carriers for a cytotoxic small molecule payload such as with the anti-CD30-MMAE ADC Brentuximab vedotin. In the case of anti-checkpoint antibodies, the only documented application in the ADC format is the Bolt Biotherapeutics anti-PDL1 carrying a TLR7/8 agonist in order to activate the innate immune system. This modality depends however on the adaptive immune system primming as there is a requirement for a pre-existing tumor antigen or the arise of random neoantigens within the tumor. The Enigmo technology complete innovation therefore relies on the direct, active induction of tumor neoantigen by the ADC payload and at the same time preserving the inhibition blocking activity of the anti-checkpoint antibody or the tumor-neutralizing activity of the anti-tumor target antibody (as in the anti-HER2 antibody for example. Thus, in order to turn non-immunogenic tumors into immunogenic ones we will make use of a series antibody drug conjugates (ADCs) or protein drug conjugates (PDCs) that target checkpoint ligands expressed on tumor cells.

These cells express a variety of checkpoint ligands including PDL-1, HVEM, Galectin 9, GITRL, B7-1 and B7-2 that in their turn will bind to antibodies and other ligands that include anti-PDL-1 (for PDL-1 binding), anti-HVEM (for HVEM binding), BTLA-FC (for HVEM-binding), TIM3-FC (for Galectin 9 binding), PD1-FC (for PDL-1 binding), CTLA-4-FC (for B7-1 and B7-2 binding) and GITR-FC (for GITRL binding). These reagents have the double function of (1) blocking the binding of the target to its counter-ligand expressed on T cells and (2) transporting the Enigmo drug payload to the target cancer cell expressing the target checkpoint ligand.

These reagents can be used either alone, for example, anti-PDL-1-Enigmo ADC against a specific tumor type expressing PDL-1 or as combinatorial engineered proteins with more the one specificity, for example, an anti-PDL-1-anti-HVEM bispecific antibody-Enigmo ADC used against a tumor expressing either PDL-1 or HVEM or both ligands. Other modalities include trispecific reagents such as anti-PDL-1-anti-HVEM-TIM3-FC-Enigmo fusion protein targeting any tumor expressing any of the target checkpoints (PDL1 or Galectin 9 or HVEM) alone or in any combination, and so on. In other instances, the Enigmo payload can be transported by an antibody to a target that is not a checkpoint ligand but is a tumor-specific target such as HER2, CEA, CD19, CD20, CD30, CD33, Nectin-4, CD22, CD38, CD79b, Trop-2, Epcam. Thus, the target tumor for Enigmo applications can be any tumor that expresses a checkpoint ligand or expresses a tumor-specific target or any combination of checkpoints and tumor-specific targets that can be addressed by any of the reagents listed above alone, or in combination, for example an anti-HER2-anti-PDL1-Enigmo bispecific antibody ADC.

Today at least half of all biological drugs in development are aimed at interfering with checkpoint inhibitors. Checkpoint therapy has achieved great clinical success in a fraction of neoplasms hitherto intractable with conventional drugs and radiation. Despite this success, only a minority fraction of cancers with positive markers of expression of checkpoint inhibitors are effectively treated with these drugs and the reason for this relatively low coverage is a direct result of the total mutation burden registered by any given cancer.

A good number of studies by several different groups have shown that in general cancers with mutation burden above 100 are treatable while those under 100 are not effectively treatable by anti-checkpoint therapy. This number is in accordance with the observations showing that on average, only 1 in 100 mutations leads to the generation of a protein fragment with an immunogenic characteristic. Therefore, the estimate is that the generation of immunogenic protein fragments from at least one immunogenic mutation is sufficient to initiate an immune response capable of controlling the growth of tumors when drugs that block checkpoints are used as therapy. Another condition that is usually observed in the case of immunotherapy is the result evolutionary pressures on the tumors, which in many cases presents a genetic or epigenetic heterogeneity that allows the tumor to up or down-regulate the expression of certain checkpoints to escape the action of a particular anti-checkpoint drug. In these cases, the most sought over strategy is to use combination checkpoint therapy or, using antibodies for multiple checkpoint receptors in the form of bispecific antibodies as a path to trying stifle the tumors escape mechanisms under these conditions. Despite all of these improvements in therapy design and reagents, the bottleneck of this therapeutic modality is the fact that most tumors do not accumulate enough mutations to become immunogenic. Embodiments of this invention address this issue.

### BRIEF SUMMARY OF THE INVENTION

In one embodiment the application discloses compounds having the structure of Formula (I):

X₂ₐ—(X₃)ₘ (I),

and pharmaceutically acceptable salts, solvates, hydrates, isomers, or tautomers thereof, wherein:
m is 1, 2, 3, 4, 5, 6, 7, 8, 9, or 10;
X₂ₐ is a linker; and
X₃ is a therapeutic compound.

In one embodiment the present disclosure provides compounds having the structure of Formula (II):

X₁—[X₂—(X₃)ₘ]ₙ (II),

and pharmaceutically acceptable salts, solvates, hydrates, isomers, or tautomers thereof, wherein:
m is 1, 2, 3, 4, 5, 6, 7, 8, 9, or 10;
n is 1, 2, 3, 4, 5, 6, 7, 8, 9, 10, or 11;
X₁ is a biologically active polypeptide or hormone;
X₂ is a linker; and
X₃ is a therapeutic compound.

In another embodiment the present disclosure provides compounds having the structure of Formula (III):

X₁—[X₂—(X₃)ₘ]ₙ (III),

and pharmaceutically acceptable salts, solvates, hydrates, isomers, or tautomers thereof, wherein:
m is 1, 2, 3, 4, 5, 6, 7, 8, 9, or 10;
n is 1, 2, 3, 4, 5, 6, 7, 8, 9, 10, or 11;
X₁ is bispecific antibodies and soluble checkpoint FC's thereof;
X₂ is a linker; and
X₃ is a therapeutic compound.

### DETAILED DESCRIPTION OF THE DISCLOSURE

Before describing selected embodiments of the present disclosure in detail, it is to be understood that the present invention is not limited to the embodiments described herein. The disclosure and description herein are illustrative and explanatory of one or more presently preferred embodiments and variations thereof, and it will be appreciated by those skilled in the art that various changes in the design, organization, means of operation, structures and location, methodology, and use of mechanical equivalents may be made without departing from the spirit of the invention.

As well, it should be understood that the diagrams are intended to illustrate and disclose presently preferred embodiments to one of skill in the art, but are not intended to be manufacturing level drawings or renditions of final products and may include simplified conceptual views to facilitate understanding or explanation. As well, the relative size and arrangement of the components may differ from that shown and still operate within the spirit of the invention.

Moreover, it will be understood that various directions such as “upper”, “lower”, “bottom”, “top”, “left”, “right”, “first”, “second” and so forth are made only concerning explanation in conjunction with the drawings, and that components may be oriented differently, for instance, during transportation and manufacturing as well as operation. Because many varying and different embodiments may be made within the scope of the concept(s) herein taught, and because many modifications may be made in the embodiments described herein, it is to be understood that the details herein are to be interpreted as illustrative and non-limiting.

The articles “a” and “an” are used in this disclosure and may refer to one or more than one (e.g., to at least one) of the grammatical object of the article. By way of example, “an element” may mean one element or more than one element.

The term “and/or” is used in this disclosure and may mean either “and” or “or” unless indicated otherwise.

Unless specifically stated, as used herein, the term “about” may refer to a range of values ±10% of a specified value. For example, the phrase “about 200” may include ±10% of 200, or from 180 to 220. When stated otherwise, the term about may refer to a range of values that include ±20%, ±10%, or ±5%, etc.

The terms “peptide,” “polypeptide,” and “protein” may be used interchangeably herein, and may refer to a polymeric form of amino acids of any length, which can include coded and non-coded amino acids, chemically or biochemically modified or derivatized amino acids, full-length polypeptides, fragments of polypeptides, variants of polypeptides, truncated polypeptides, fusion polypeptides, or polypeptides having modified peptide backbones. The polypeptides disclosed herein may also be variants differing from a specifically recited “reference” polypeptide (e.g., a wild-type polypeptide) by amino acid insertions, deletions, mutations, and/or substitutions.

Historically, attempts to make the tumor immunogenic relied on technologies based on viral infection of tumor cells to display viral antigens (Oncolytics) or through vaccination using modified cancer cells expressing immunomodulatory cytokines (GVAX) in an attempt to breakdown tolerance to self-antigens expressed by the tumor. These are at several stages of clinical development. Recent developments included the use of ADCs consisting of an anti-tumor antibody (e.g. anti-HER2 or anti-CD30) and a linked small molecule payload consisting of a TLR agonist in order to stimulate the innate immune system to present tumor antigens in a immunologically relevant context (Bolt Therapeutics). With the exception of the Oncolytics approach the vaccine and the ADC approaches are not technically attempting to make the tumor immunogenic because this requires the generation of new antigens by modification of self which is not facilitated by such therapies. The tumor viral infection approach has the drawback that every tumor cell needs to be infected by the virus, a very difficult task for a solid tumor and that all healthy cells do not get infected. This requires the engineering of viruses with special conditional genetics that will vary according to tumor type and individuals. For example, virus that can only establish an infection in p53 loss-of-function tumors. This is what makes this approach more a matter of personalized medicine as in the case with the CAR-T immunotherapy.

In one embodiment, the unique position includes our initiative in associating the anti-checkpoint reagents with the small molecules of the Enigmo class in a drug of the ADC class (Antibody-drug conjugates). Preliminary data (see below) was generated in a melanoma model in mice with Enigmo class ADCs anti-checkpoint antibodies. It is noted that the tumor when expressing a proven exogenous immunogenic protein (OVA) is rejected by the recipient animal when the animal is previously immunized against OVA. However, if the tumor also expresses checkpoint inhibitors together with OVA, there is no rejection of the tumor, exemplifying the classic case of immune escape by the tumor via increased expression of checkpoints. In this case, treatment with antibodies for checkpoint significantly restores tumor rejection. However, the response varies depending on the identity of the checkpoint expressed together with OVA.

In wild type tumors the growth is continuous, and the animal succumbs to the disease in a few weeks. Some checkpoints are expressed at baseline levels that are almost undetectable in these tumors and the use of anti-checkpoints has no impact on tumor growth. However, in a striking way, in tumors expressing high levels of checkpoint markers, but with wild-type immunogenic content, the action of anti-checkpoints is practically null, exemplifying another classic scenario where tumors express high levels of checkpoints, but do not have enough mutation burden to generate immunogenic fragments and therefore fail anti-checkpoint therapy.

The application of the anti-checkpoint-enigmo drug induces tumor rejection with high efficacy and this rejection is attributable to the immune response by CD4 and CD8 T cells since the previous elimination of these cells from the recipient completely abolishes rejection by the anti-checkpoint-enigmo proving that the enigmo drugs induce the expression of proteins with altered amino acid sequences in sufficient number to cause immunogenicity and consequent immunological rejection. In short, the anti-checkpoint-enigmo drug makes non-reactive tumor therapy into a reactive and highly treatable tumor therapy, potentially recovering the pool of the greater proportion of patients who would be non-reactive despite the high expression of checkpoint markers. Embodiments of this invention has the potential to treat any tumor, in any person in whom the tumor expresses checkpoint inhibitors or any tumor specific antigen that can be targeted by a ligand such as an antibody or soluble receptor, etc. carrying an enigmo payload.

One embodiment includes a class of molecules that generally causes an expansion of the genetic coding sequences, that is, they increase either the number of ORFs (open reading frames) read by the ribosome from a single species of messenger RNA or the length of the ORF. Alternatively, some variants of this class cause mutations in the genetic code and consequently the production of proteins with altered amino acid sequences in relation to the wild type. Although such alterations are normally undesirable due to their pro-tumorigenic potential (think of U.V and gamma-type radiation) they are also sources of inexhaustible immunological variability and it is this characteristic that defines the revolutionary rationale of this concept.

In some embodiments, conservative substitutions may be made in the amino acid sequence of a polypeptide without disrupting the three-dimensional structure or function of the polypeptide. Conservative substitutions may be accomplished by the skilled artisan by substituting amino acids with similar hydrophobicity, polarity, and R-chain length for one another. Additionally, by comparing aligned sequences of homologous proteins from different species, conservative substitutions may be identified by locating amino acid residues that have been mutated between species without altering the basic functions of the encoded proteins. The term “conservative amino acid substitution” may refer to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polypeptide has a certain percent “sequence identity” to another polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world wide web at sites including ncbi.nlm.nili.gov/BLAST,ebi.ac.uk/Tools/msa/tcoffee/ebi.ac.uk/Tools/msa/muscle/mafft.cbrc.jp/alignment/software/. See, e.g., Altschul et al. (1990), J. Mol. Biol. 215:403-10. This reference is incorporated by reference in its entirety.

“Optionally substituted” may refer to the replacement of hydrogen with a monovalent or divalent radical. Suitable substitution groups include, for example, hydroxyl, nitro, amino, imino, cyano, halo, thio, thioamido, amidino, oxo, oxamidino, methoxamidino, imidino, guanidino, sulfonamido, carboxyl, formyl, lower alkyl, halo lower alkyl, lower alkoxy, halo lower alkoxy, lower alkoxyalkyl, alkylcarbonyl, arylcarbonyl, aralkylcarbonyl, heteroarylcarbonyl, heteroaralkylcarbonyl, alkylthio, aminoalkyl, cyanoalkyl, and the like as defined herein. The substitution group can itself be substituted. The group substituted onto the substitution group can be, for example, carboxyl, halo; nitro, amino, cyano, hydroxyl, lower alkyl, lower alkoxy, aminocarbonyl, —SR, thioamido, —SO₃H, —SO₂R or cycloalkyl, where R is typically hydrogen, hydroxyl or lower alkyl. When the substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substitutents can be straight chain, branched or cyclic arrangements of covalently bonded carbon or heteroatoms.

“Lower alkyl” as used herein may refer to branched or straight chain alkyl groups comprising one to ten carbon atoms that independently are unsubstituted or substituted, e.g., with one or more halogen, hydroxyl or other groups. Examples of lower alkyl groups include, but are not limited to, methyl, ethyl, propyl, isopropyl, n-butyl, t-butyl, n-hexyl, neopentyl, trifluoromethyl, pentafluoroethyl, and the like.

“Alkylenyl” may refer to a divalent straight chain or branched chain saturated aliphatic radical having from 1 to 20 carbon atoms. Typical alkylenyl groups employed in compounds of the present disclosure are lower alkylenyl groups that have from 1 to about 6 carbon atoms in their backbone.

“Alkenyl” may refer herein to straight chain, branched, or cyclic radicals having one or more double bonds and from 2 to 20 carbon atoms.

“Alkynyl” may refer herein to straight chain, branched, or cyclic radicals having one or more triple bonds and from 2 to 20 carbon atoms.

“Halo lower alkyl” may refer to a lower alkyl radical substituted with one or more halogen atoms.

“Lower alkoxy” as used herein may refer to RO— wherein R is lower alkyl. Representative examples of lower alkoxy groups include methoxy, ethoxy, t-butoxy, trifluoromethoxy and the like.

“Lower alkythio” as used herein may refer to RS— wherein R is lower alkyl.

“Alkoxyalkyl” refers to the group -alk₁-O-alk₂, where alk₁ is alkylenyl or alkenyl, and alk₂ is alkyl or alkenyl.

“Lower alkoxyalkyl” refers to an alkoxyalkyl as defined herein, where alk₁ is lower alkylenyl or lower alkenyl, and alk₂ is lower alkyl or lower alkenyl.

“Aryloxyalkyl” refers to the group alkylenyl-O-aryl.

“Aralkoxyalkyl” refers to the group alkylenyl-O-aralkyl, where aralkyl is a lower aralkyl.

“Cycloalkyl” refers to a monoor polycyclic, lower alkyl substituent. Typical cycloalkyl substituents have from 3 to 8 backbone (i.e., ring) atoms in which each backbone atom is optionally substituted carbon. When used in context with cycloalkyl substituents, the term polycyclic refers herein to fused, nonfused cyclic carbon structures and spirocycles. Examples of cycloalkyl groups include, but are not limited to, cyclopropyl, cyclobutyl, cyclopentyl, cyclohexyl, adamantyl, bornyl, norbornyl, and the like.

“Cycloheteroalkyl” refers herein to cycloalkyl substituents that have from 1 to 5, and more typically from 1 to 4 heteroatoms (i.e., non-carbon atoms such as nitrogen, sulfur, and oxygen) in the ring structure, with the balance of atoms in the ring being optionally substituted carbon. Representative heterocycloalkyl moieties include, for example, morpholino, piperazinyl, piperidinyl, pyrrolidinyl, methylpryolidinyl, pyrrolidinone-yl, and the like.

“(Cycloalkyl)alkyl” and “(cycloheteroalkyl)alkyl” refer to alkyl chains substituted with cycloalkyl and cycloheteroalkyl groups respectively.

“Haloalkoxy” refers to an alkoxy radical substituted with one or more halogen atoms. The term “halo lower alkoxy” refers to a lower alkoxy radical substituted with one or more halogen atoms.

“Halo” refers herein to a halogen radical, such as fluorine, chlorine, bromine, or iodine.

“Aryl” refers to monocyclic and polycyclic aromatic groups, or fused ring systems having at least one aromatic ring, having from 3 to 14 backbone carbon atoms. Examples of aryl groups include without limitation phenyl, naphthyl, dihydronaphtyl, tetrahydronaphthyl, and the like.

“Aralkyl” refers to an alkyl group substituted with an aryl group. Typically, aralkyl groups employed in compounds of the present disclosure have from 1 to 6 carbon atoms incorporated within the alkyl portion of the aralkyl group. Suitable aralkyl groups employed in compounds of the present disclosure include, for example, benzyl, picolyl, and the like.

“Heteroaryl” refers herein to aryl groups having from one to four heteroatoms as ring atoms in an aromatic ring with the remainder of the ring atoms being aromatic or non-aromatic carbon atoms. When used in connection with aryl substituents, the term polycyclic refers herein to fused and non-fused cyclic structures in which at least one cyclic structure is aromatic, such as, for example, benzodioxozolo, naphthyl, and the like. Exemplary heteroaryl moieties employed as substituents in compounds of the present disclosure include pyridyl, pyrimidinyl, thiazolyl, indolyl, imidazolyl, oxadiazolyl, tetrazolyl, pyrazinyl, triazolyl, thiophenyl, furanyl, quinolinyl, purinyl, benzothiazolyl, benzopyridyl, and benzimidazolyl, and the like.

“Amino” refers herein to the group —NH₂. The term “lower alkylamino” refers herein to the group —NRR¹ where R and R¹ are each independently selected from hydrogen or lower alkyl. The term “arylamino” refers herein to the group —NRR¹ where R is aryl and R¹ is hydrogen, lower alkyl, aryl, or aralkyl. The term “aralkylamino” refers herein to the group —NRR¹ where R is aralkyl and R¹ is hydrogen, lower alkyl, aryl, or aralkyl. The terms “heteroarylamino” and “heteroaralkylamino” are defined by analogy to arylamino and aralkylamino.

“Aminocarbonyl” refers herein to the group —C(O)NH₂. The terms “lower alkylaminocarbonyl,” “arylaminocarbonyl,” “aralkylaminocarbonyl,” “heteroarylaminocarbonyl,” and “heteroaralkylaminocarbonyl” refer to —C(O)NRR¹ where R and R¹ independently are hydrogen and optionally substituted lower alkyl, aryl, aralkyl, heteroaryl, and heteroaralkyl respectively by analogy to the corresponding terms herein.

“Thio” refers to —SH. The terms lower alkylthio, arylthio, heteroarylthio, cycloalkylthio, cycloheteroalkylthio, aralkylthio, heteroaralkylthio, (cycloalkyl)alkylthio, and (cycloheteroalkyl)alkylthio refer to —SR, where R is optionally substituted lower alkyl, aryl, heteroaryl, cycloalkyl, cycloheteroalkyl, aralkyl, heteroaralkyl, (cycloalkyl)alkyl, and (cycloheteroalkyl)alkyl respectively.

“Sulfonyl” refers herein to the group —SO₂—. The terms lower alkylsulfonyl, arylsulfonyl, textitheteroaryl sulfonyl, cycloalkyl sulfonyl, cycloheteroalkyl sulfonyl, aralkyl sulfonyl, heteroaralkylsulfonyl, (cycloalkyl)alkylsulfonyl, and (cycloheteroalkyl)alkylsulfonyl refer to —SO₂R where R is optionally substituted lower alkyl, aryl, heteroaryl, cycloalkyl, cycloheteroalkyl, aralkyl, heteroaralkyl, (cycloalkyl)alkyl, and (cycloheteroalkyl)alkyl respectively.

“Sulfinyl” refers herein to the group —SO—. The terms lower alkylsulfinyl, arylsulfinyl, heteroarylsulfinyl, cycloalkyl sulfinyl, cycloheteroalkyl sulfinyl, aralkyl sulfinyl, heteroaralkylsulfinyl, (cycloalkyl)alkylsulfinyl, and (cycloheteroalkyl)alkylsulfinyl refer to —SOR where R is optionally substituted lower alkyl, aryl, heteroaryl, cycloalkyl, cycloheteroalkyl, aralkyl, heteroaralkyl, (cycloalkyl)alkyl, and (cycloheteroalkyl)alkyl respectively.

“Nitrilo” refers to —CN.

“Formyl” refers to —C(O)H.

“Carboxyl” refers to —C(O)OH.

“Carbonyl” refers to the divalent group —C(O)—. The terms lower alkylcarbonyl, arylcarbonyl, heteroarylcarbonyl, cycloalkylcarbonyl, cycloheteroalkylcarbonyl, aralkycarbonyl, heteroaralkylcarbonyl, (cycloalkyl)alkylcarbonyl, and (cycloheteroalkyl)alkylcarbonyl refer to —C(OR)—, where R is optionally substituted lower alkyl, aryl, heteroaryl, cycloalkyl, cycloheteroalkyl, aralkyl, heteroaralkyl, (cycloalkyl)alkyl, and (cycloheteroalkyl)alkyl respectively.

“Thiocarbonyl” refers to the group —C(S)—. The terms lower alkylthiocarbonyl, arylthiocarbonyl, heteroarylthiocarbonyl, cycloalkylthiocarbonyl, cycloheteroalkylthiocarbonyl, aralkylthiocarbonyloxlthiocarbonyl, heteroaralkylthi ocarbonyl, (cycloalkyl)alkylthiocarbonyl, and (cycloheteroalkyl)alkylthiocarbonyl refer to —C(S)R—, where R is optionally substituted lower alkyl, aryl, heteroaryl, cycloalkyl, cycloheteroalkyl, aralkyl, heteroaralkyl, (cycloalkyl)alkyl, and (cycloheteroalkyl)alkyl respectively.

“Carbonyloxy” refers generally to the group —C(O)O—. The terms lower alkylcarbonyloxy, arylcarbonyloxy, heteroaryl carbonyloxy, cycloalkylcarbonyl oxy, cycloheteroalkylcarbonyloxy, aralkylcarbonyloxy, heteroaralkyl carbonyloxy, (cycloalkyl)alkylcarbonyloxy, (cycloheteroalkyl)alkylcarbonyloxy refer to —C(O)OR, where R is optionally substituted lower alkyl, aryl, heteroaryl, cycloalkyl, cycloheteroalkyl, aralkyl, heteroaralkyl, (cycloalkyl)alkyl, and (cycloheteroalkyl)alkyl respectively.

“Oxycarbonyl” refers to the group —OC(O)—. The terms lower alkyloxycarbonyl, aryloxycarbonyl, heteroaryloxycarbonyl, cycloalkyloxycarbonyl, cycloheteroalkyloxycarbonyl, aralkyloxycarbonyloxycarbonyl, heteroaralkyloxycarbonyl, (cycloalkyl)alkyloxycarbonyl, (cycloheteroalkyl)alkyloxycarbonyl refer to —OC(O)R, where R is optionally substituted lower alkyl, aryl, heteroaryl, cycloalkyl, cycloheteroalkyl, aralkyl, heteroaralkyl, (cycloalkyl)alkyl, and (cycloheteroalkyl)alkylrespectively.

“Carbonylamino” refers to the group —NHC(O)—. The terms lower alkylcarbonylamino, arylcarbonylamino, heteroarylcarbonylamino, cycloalkylcarbonylamino, cycloheteroalkylcarbonylamino, aralkylcarbonylamino, heteroaralkylcarbonylamino, (cycloalkyl)alkylcarbonylamino, and (cycloheteroalkyl)alkylcarbonylamino refer to —NHC(O)R—, where R is optionally substituted lower alkyl, aryl, heteroaryl, cycloalkyl, cycloheteroalkyl, aralkyl, heteroaralkyl, (cycloalkyl)alkyl, or (cycloheteroalkyl)alkyl respectively. In addition, the present disclosure includes n-substituted carbonylamino (—NR¹C(O)R), where R¹ is optionally substituted lower alkyl, aryl, heteroaryl, aralkyl, or heteroaralkyl and R retains the previous defintion.

“Carbonylthio” refers to the group —C(O)S—. The terms lower alkylcarbonylthio, arylcarbonylthio, heteroarylcarbonylthio, cycloalkylcarbonylthio, cycloheteroalkylcarbonylthio, aralkylcarbonylthio, heteroaralkylcarbonylthio, (cycloalkyl)alkylcarbonylthio, (cycloheteroalkyl)alkylcarbonylthio refer to —C(O)SR, where R is optionally substituted lower alkyl, aryl, heteroaryl, cycloalkyl, cycloheteroalkyl, aralkyl, heteroaralkyl, (cycloalkyl)alkyl, and (cycloheteroalkyl)alkyl respectively.

“Guanidino” or “Guanidyl” refers to moieties derived from guanidine, H₂N—C(═NH) —NH₂. Such moieties include those bonded at the nitrogen atom carrying the formal double bond (the 2-position of the guanidine, e.g., diaminomethyleneamino, ((H₂N)₂—C═NH—) and those bonded at either of the nitrogen atoms carrying a formal single bond (the 1 or 3-positions of the guanidine, e.g., H₂NC(═NH)—NH—). The hydrogen atoms at either nitrogen can be replaced with a suitable substituent, such as lower alkyl, aryl, or lower aralkyl.

“Amidino” refers to the moieties R—C(═N)—NR¹— (the radical being at the N¹ nitrogen) and R—(NR¹)CN— (the radical being at the N² nitrogen), where R and R¹ can be hydrogen, lower alkyl, aryl, or lower aralkyl.

“Imino” refers to the group —C(═NR)—, where R can be hydrogen or optionally substituted lower alkyl, aryl, heteroaryl, or heteroaralkyl respectively. The terms “imino lower alkyl,” “iminocycloalkyl,” “iminocycloheteroalkyl,” “iminoaralkyl,” “iminoheteroaralkyl,” “(cycloalkyl)iminoalkyl,” “(cycloiminoalkyl)alkyl,” “(cycloiminoheteroalkyl)alkyl,” and “(cycloheteroalkyl)iminoalkyl” refer to optionally substituted lower alkyl, cycloalkyl, cycloheteroalkyl, aralkyl, heteroaralkyl, (cycloalkyl)alkyl, and (cycloheteroalkyl)alkyl groups that include an imino group, respectively.

“Oximino” refers to the group —C(═NOR)—, where R can be hydrogen (“hydroximino”) or optionally substituted lower alkyl, aryl, heteroaryl, or heteroaralkyl respectively. The terms “oximino lower alkyl,” “oximinocycloalkyl,” “oximinocycloheteroalkyl,” “oximinoaralkyl,” “oximinoheteroaralkyl,” “(cycloalkyl)oximinoalkyl,” “(cyclooximinoalkyl)alkyl,” “(cyclooximinoheteroalkyl)alkyl,” and “(cycloheteroalkyl)oximinoalkyl” refer to optionally substituted lower alkyl, cycloalkyl, cycloheteroalkyl, aralkyl, heteroaralkyl, (cycloalkyl)alkyl, and (cycloheteroalkyl)alkyl groups that include an oximino group, respectively.

“Methylene” as used herein refers to an unsubstituted, monosubstituted, or disubstituted carbon atom having a formal sp³ hybridization (i.e., —CRR¹—, where R and R¹ are hydrogen or independent substituents).

“Methine” as used herein refers to an unsubstituted or substituted carbon atom having a formal sp² hybridization (i.e., CR═ or ═CR—, where R is hydrogen or a substituent).

### Compounds of the Disclosure

The present disclosure relates to compounds (e.g., Formulae II and III), or pharmaceutically acceptable salts, hydrates, solvates, isomers, or tautomers thereof, useful for the treatment of disorders, including but not limited to pancreatic cancer and other check point positive cancers. More particularly, the compounds of the disclosure (e.g., Formulae II and III), or pharmaceutically acceptable salts, hydrates, solvates, isomers, or tautomers thereof, may comprise biologically active polypeptides or hormones modified to include the attachment of therapeutic compounds using linkers. The compounds of the disclosure, or pharmaceutically acceptable salts, hydrates, solvates, isomers, or tautomers thereof, also comprise therapeutic compounds connected to linkers.

In an aspect, the present disclosure provides compounds having the structure of Formula (I):

X₂ₐ—(X₃)ₘ (I),

and pharmaceutically acceptable salts, solvates, hydrates, isomers, or tautomers thereof, wherein:
m is 1, 2, 3, 4, 5, 6, 7, 8, 9, or 10;
X₂ₐ is a linker; and
X₃ is a therapeutic compound.

As shown in Formula (I), multiple X₃ moieties, the number defined by m, may be attached to X₂ₐ.

In some embodiments of the compound of Formula (I), m is 1. In some embodiments of the compound of Formula (I), m is 2, 3, 4, 5, 6, 7, or 8. In some embodiments of the compound of Formula (I), m is 1, 2, or 3. In some embodiments of the compound of Formula (I), m is 2, 3, 4, or 5. In some embodiments of the compound of Formula (I), m is 2. In some embodiments of the compound of Formula (I), m is 3. In some embodiments of the compound of Formula (I), m is 4. In some embodiments of the compound of Formula (I), m is 5. In some embodiments of the compound of Formula (I), m is 6. In some embodiments of the compound of Formula (I), m is 7. In some embodiments of the compound of Formula (I), m is 8. In some embodiments of the compound of Formula (I), m is 9. In some embodiments of the compound of Formula (I), m is 10. In some embodiments, m is an integer between 1 and 10.

X₂ₐ is a linker, which can take many forms as shown herein. In some embodiments of the compound of Formula (I), X₂ₐ is a linker which has not yet been reacted with a biologically active polypeptide or hormone. In some embodiments of the compound of Formula (I), X₂ₐ is a cleavable linker. In some embodiments of the compound of Formula (I), X₂ₐ is a non-cleavable linker. As used herein, a “cleavable linker” may refer to a linker comprising a lysosomal and/or endosomal-specific enzyme cleavage site, such as a β-glucuronidase site, a β-galactosidase site, or a cathepsin site. With a cleavable linker, the therapeutic compound may be liberated only after internalization of the conjugate by the cell, in proximity to the therapeutic compound's intracellular target. This targeted liberation may enable use of toxic, potent therapeutic compounds for treatment of disorders. “Non-cleavable linkers” may refer to linkers conjugated to therapeutic compounds that will act directly absent release.

In some embodiments of the compound of Formula (I), X₂ₐ is a linker listed in Table 1, wherein the right side of X₂ₐ, as drawn, is bound to X₃. In Table 1, R groups=H, glycol ether, or an additional glycol linker alkyl, aryl, and arylalkyl, alkynes, alkenes, substituted phenyl groups, aromatic heterocycles, glycol ether, or an additional glycol linker attaching to the therapeutic compound.

TABLE 1
Exemplary X₂ₐ Linker Linkers

| Compound | |
|---|---|
| Number | Structure |
| L-1 Cleavable Linker | 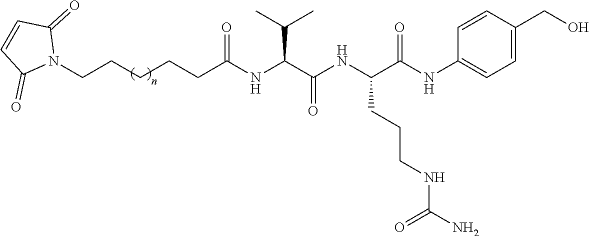 |
| L-2 Cleavable Linker | 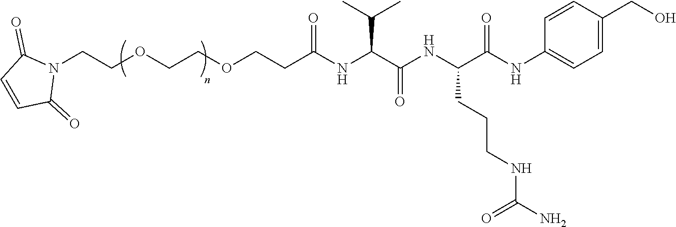 |
| L-3 Cleavable Linker | 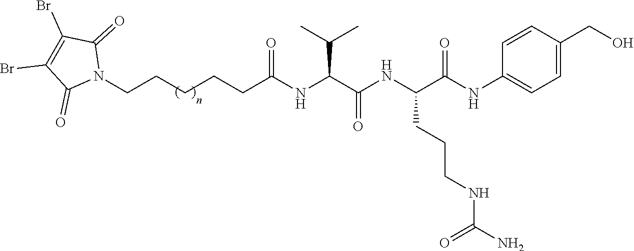 |
| L-4 Cleavable Linker | 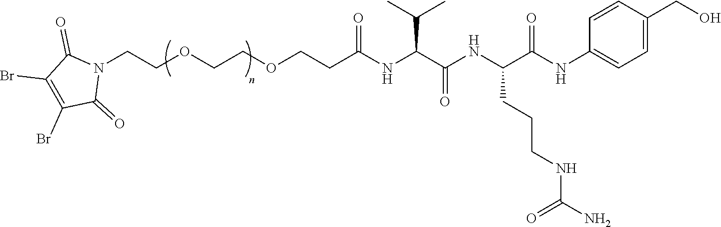 |
| L-5 Cleavable Linker | 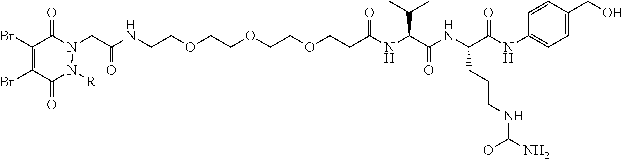 |
| L-6 Cleavable Linker | 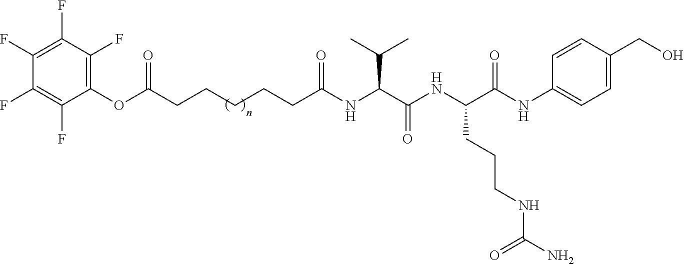 |
| L-7 Cleavable Linker | 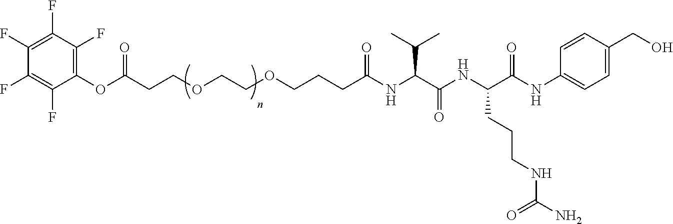 |
| L-8 Cleavable Linker | 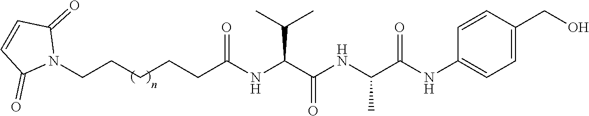 |
| L-9 Cleavable Linker | 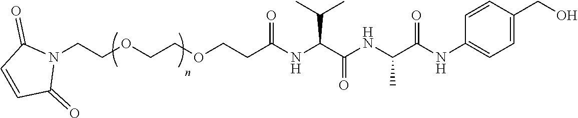 |
| L-10 Cleavable Linker | 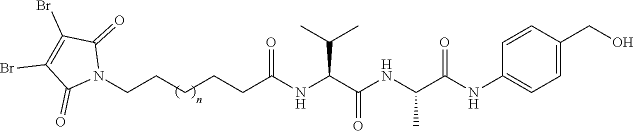 |
| L-11 Cleavable Linker | 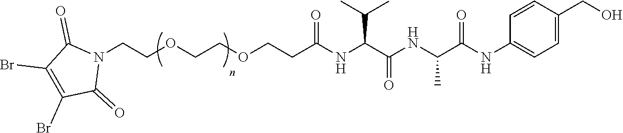 |
| L-12 Cleavable Linker | 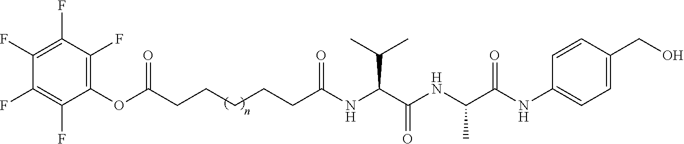 |
| L-13 Cleavable Linker | 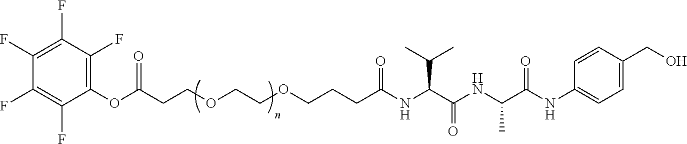 |
| L-14 Cleavable Linker | 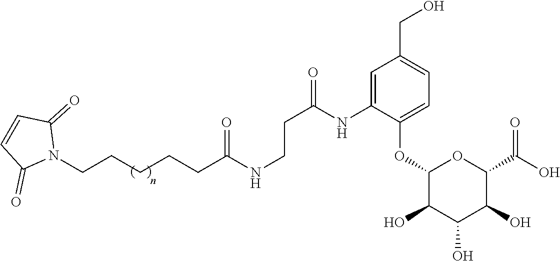 |
| L-15 Cleavable Linker | 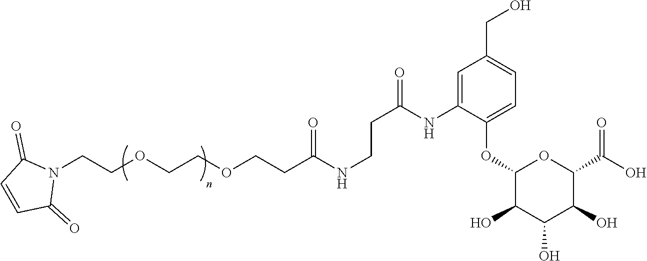 |
| L-16 Cleavable Linker | 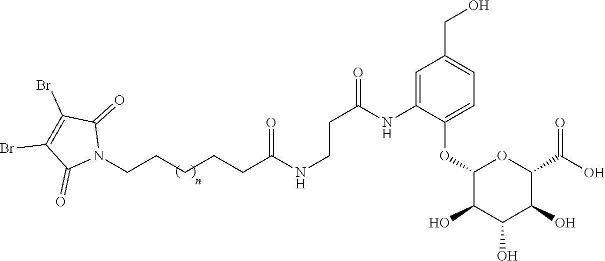 |
| L-16 Cleavable Linker | 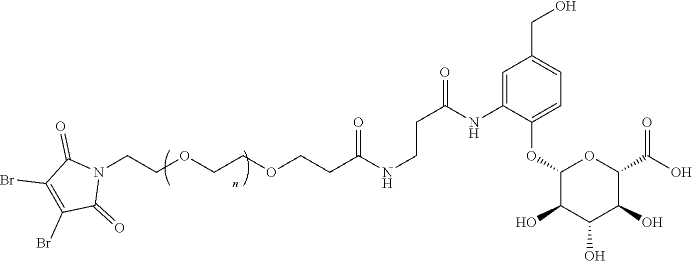 |
| L-17 Cleavable Linker | 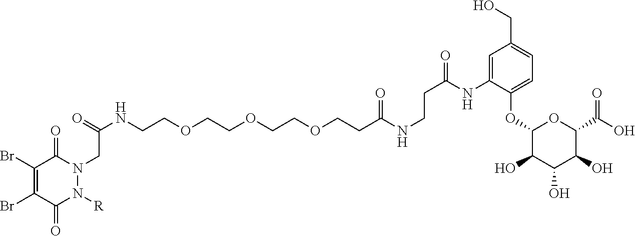 |
| L-18 Cleavable Linker | 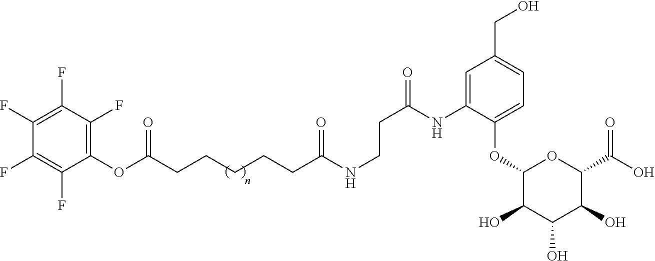 |
| L-19 Cleavable Linker | 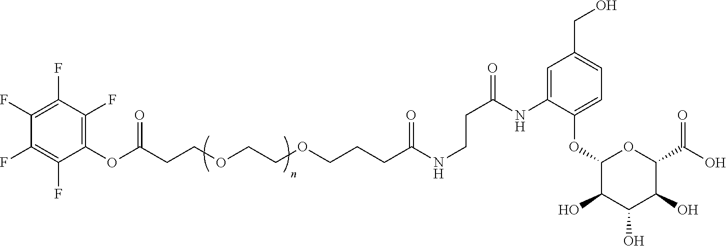 |
| L-20 Non- Cleavable Linker | 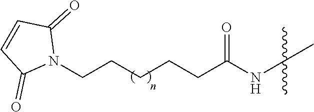 |
| L-21 Non- Cleavable Linker | 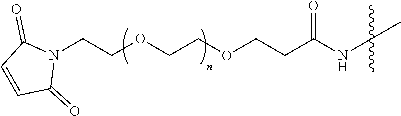 |
| L-22 Non- Cleavable Linker | 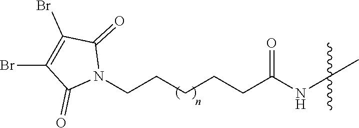 |
| L-23 Non- Cleavable [symbol] | 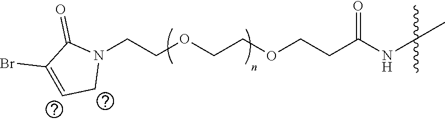 |
| L-24 Non- Cleavable Linker | 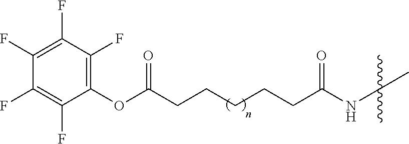 |
| L-25 Non- Cleavable Linker | 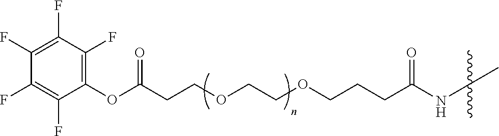 |

[symbol]

indicates data missing or illegible when filed

The Enigmo class of small molecule payloads can be classified in three groups, according to the proposed mechanism of action of each group.

In Group 1 are the small molecules that cause mutagenesis in DNA through intercalation. Especially those that cause a type of mutation called a transversion, a point mutation in DNA in which a single (two ring) purine (A or G) is changed for a (one ring) pyrimidine (T or C), or vice versa. Amongst them, the acridine dye series derivative Quinacrine is preferred given its long clinical history as an anti-malarial and anti-Lupus drug. Others are acridine orange, acridine yellow and proflavine. These transversions have the potential to change the DNA coding sequence and therefore the encoded proteins primary amino acid sequence, therefore a potential source of new antigenic targets.

In Group 2 are the small molecules that bind RNA and decrease the ribosome fidelity for the AUG start codon, allowing for alternative start codons such as UUG that may reside either upstream or downstream of the canonical AUG site. Consequently, potential upstream open reading frames are translated and are a source of new protein amino acid sequences with antigenic potential. Likewise, downstream initiation sites will produce truncated proteins that may not fold properly or assume conformations that allow for differential proteolysis as compared to the wild-type protein, potentially generating new antigenic epitopes. Additionally, initiation at non-AUG sites in a different reading frame will generate a completely different primary protein sequence and therefore a source of completely new antigens. Two small molecules have been identified with such properties, Isoquinoline-1-carboxylic acid and 7-amino-5-iodo-8 quinolinol hydrochloride. In addition, other isoquinoline alkaloids such as sanguinarine, berberine and coralline have the potential of being effective in this setting given their unique interaction capability with poly(A) mRNA tails.

In Group 3 are the small molecules that suppress stop codon-induced termination of RNA translation, promoting ribosomal stop codon read-trough. These will promote the biosynthesis of proteins with never before expressed C-terminal extensions that can potentially span hundreds of amino acids, therefore a potential source of completely new antigens. Examples are the aminoglycosides family, including G418 and gentamicin.

Examples of therapeutic compounds, or pharmaceutically acceptable salts, solvates, hydrates, isomers, or tautomers thereof, useful in the disclosure are detailed in Table 2. In Table 2, R₁=H, alkyl, aryl, arylalkyl, arylalkyne, arylalkene, heterocyclic, heteroaryl, substituted phenyl groups, aromatic heterocycles, glycol ether, or an additional glycol linker. R₂ groups=H, alkyl, aryl, and arylalkyl, alkynes, alkenes, substituted phenyl groups, aromatic heterocycles, glycol ether, or an additional glycol linker and the like.

TABLE 2
Exemplary Therapeutic Compounds for X₃

| Compound Name | Drug ID | Structure | Activity |
|---|---|---|---|
| N4-(6-chloro-2- methoxyacridin-9-yl)- N1,N1-diethylpentane- 1,4-diamine dihydrochloride | Quinacrine | 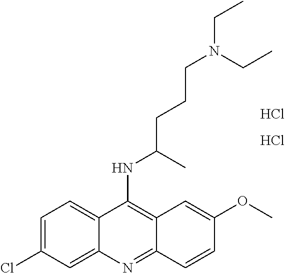 [SMILES: CCN(CC)CCCC(C)Nc1c2ccc(Cl)cc2nc2ccc(OC)cc12 ; Cl ; Cl] | Mutagen |
| 1-((2-(diethylamino) ethyl)amino)-4- (hydroxymethyl)-9H- thioxanthen-9- one | Hycanthone | 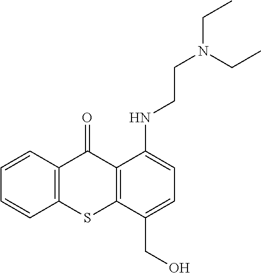 [SMILES: CCN(CC)CCNc1ccc(CO)c2sc3ccccc3c(=O)c12] | Mutagen |
| N3,N3,N6,N6- tetramethylacridine- 3,6-diamine hydrochloride | Acridine Orange | 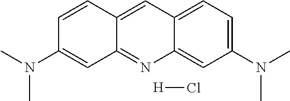 | Mutagen |
| 2,7-dimethylacridine- 3,6-diamine | Acridine Yellow | 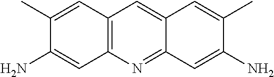 | Mutagen |
| acridine-3,6-diamine | Proflavin | 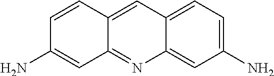 [SMILES: Nc1ccc2cc3ccc(N)cc3nc2c1] | Mutagen |
| Isoquinoline-1- carboxylic Acid | Isoquinolinic Acid | 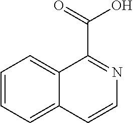 [SMILES: O=C(O)c1nccc2ccccc12] | Shift-Frame Mutants |
| 7-Amino-5-iodo- 8-quinolinol | 7-Amino-5- iodo- quinolinol | 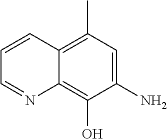 | Shift-Frame Mutants |
| 13-methyl- [1,3]dioxolo[4′,5′:4,5] benzo[1,2- c][1,3]dioxolo[4,5-i] phenanthridin-13 ium | Sanguinarine | 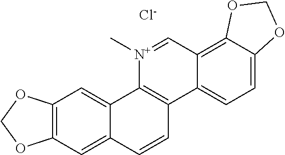 [SMILES: C[n+]1cc2c3c(ccc2c2ccc4cc5c(cc4c21)OCO5)OCO3 ; [Cl-]] | Shift-Frame Mutants |
| 9,10-dimethoxy- 5,6-dihydro- [1,3]dioxolo[4,5-g] isoquinolino[3,2- a]isoquinolin-7-ium | Berberine | 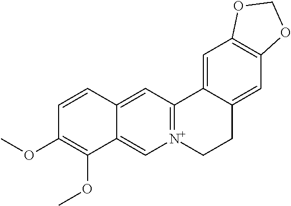 [SMILES: COc1ccc2cc3[n+](cc2c1OC)CCc1cc2c(cc1-3)OCO2] | Shift-Frame Mutants |
| 4-(bis(4- hydroxyphenyl) methylene)cyclohexa- 2,5-dien-1-one | Corallin | 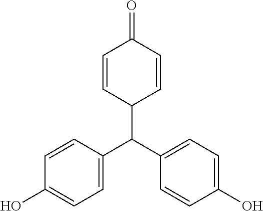 [SMILES: O=C1C=CC(C(c2ccc(O)cc2)c2ccc(O)cc2)C=C1] | Shift-Frame Mutants |
| (2R,3R,5R)-2- (((1S,2S,3R,4S)-4,6- diamino-3-(((2R,3R,6S)- 3-amino-6- (2-aminoethyl) tetrahydro-2H-pyran- 2-yl)oxy)-2- hydroxycyclohexyl)oxy)- 5-methyl-4- (methylamino)tetrahydro- 2H-pyran-3,5-diol | Gentamicin Sulfate | 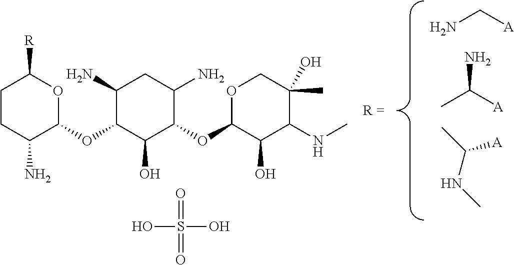 | Stop Codon Suppressor |
| (2R,3S,4R,5R,6S)- 5-amino-6- (((1R,2S,3S,4R,6S)- 4,6-diamino-3- (((2R,3R,4R,5R)- 3,5-dihydroxy-5- methyl-4-(methylamino) tetrahydro- 2H-pyran-2-yl)oxy)-2- hydroxycyclohexyl) oxy)-2-((R)-1- hydroxyethyl) tetrahydro-2H-pyran- 3,4-diol sulfate | G418 | 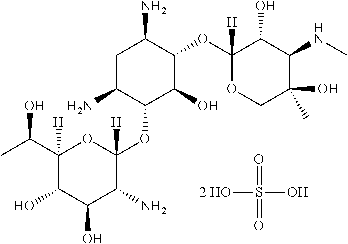 | Stop Codon Suppressor |
| N-(5-amino-3-(2- aminoundecanamido) pentanamido)- N-methylglycine | TCP-1109 | 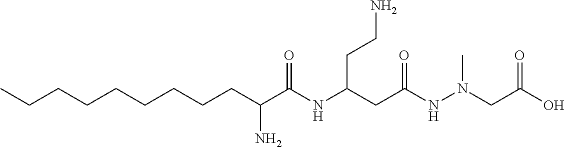 [SMILES: CCCCCCCCCC(N)C(=O)NC(CCN)CC(=O)NN(C)CC(=O)O] | Stop Codon Suppressor |
| 4-(benzo[d][1,3]dioxol- 5-ylmethyl)-N- (3-ethoxypropyl) piperazine-1- carbothioamide | GJ071 | 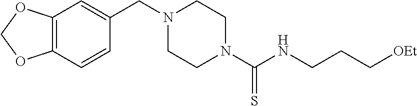 [SMILES: CCOCCCNC(=S)N1CCN(Cc2ccc3c(c2)OCO3)CC1] | Stop Codon Suppressor |
| N-(4-fluorophenyl)- 2-((4-(3- methoxyphenyl)-5- (pyridin-2-yl)-4H- 1,2,4-triazol-3-yl) thio)acetamide | GJ072 | 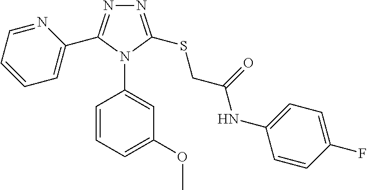 [SMILES: COc1cccc(-n2c(SCC(=O)Nc3ccc(F)cc3)nnc2-c2ccccn2)c1] | Stop Codon Suppressor |
| 2-amino-7-isopropyl- 5-oxo-5H- chromeno[2,3-b] pyridine-3- carboxylic acid | Amlexanox | 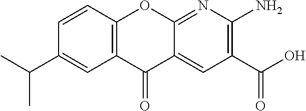 [SMILES: CC(C)c1ccc2oc3nc(N)c(C(=O)O)cc3c(=O)c2c1] | Stop Codon Suppressor |
| (2S,3R,4S,5R)-2- ((6-amino-5- nitropyrimidin-4- yl)amino)-5- (hydroxymethyl) tetrahydrofuran-3,4- diol | Clitocine | 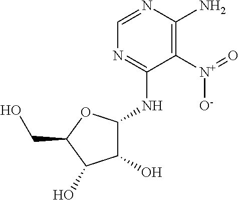 [SMILES: Nc1ncnc(N[C@H]2O[C@H](CO)[C@@H](O)[C@H]2O)c1[N+](=O)[O-]] | Stop Codon Suppressor |
| 7H-purine-2,6-diamine | DAP | 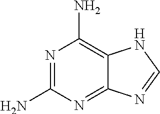 [SMILES: Nc1nc(N)c2[nH]cnc2n1] | Stop Codon Suppressor |
| (E)-4-(tert-butyl)-3-(3- nitrostyryl)phenol | NV2445 | 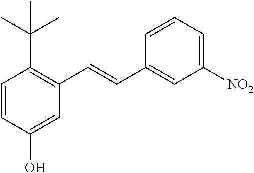 [SMILES: CC(C)(C)c1ccc(O)cc1/C=C/c1cccc([N+](=O)[O-])c1] | Stop Codon Suppressor |

In some embodiments, the compound of Formula (I) is a compound listed in Table 3, or a pharmaceutically acceptable salt, solvate, hydrate, isomer, or tautomer thereof. In Table 3, R groups=H, alkyl, aryl, and arylalkyl, alkynes, alkenes, substituted phenyl groups, aromatic heterocycles, glycol ether, or an additional glycol linker attaching to the therapeutic compound; and p is 1, 2, 3, 4, 5, or 6.

TABLE 3
Exemplary Compounds of Formula (I)

| Com- | |
|---|---|
| pound | Structure |
| A-1 | 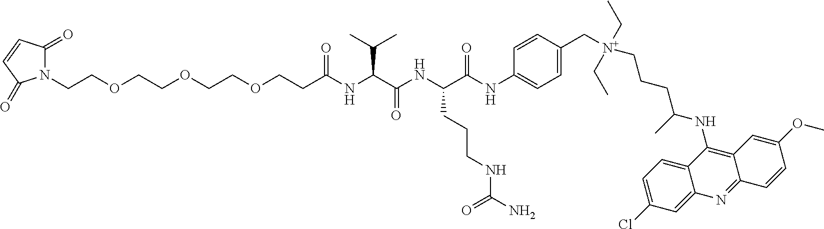 [SMILES: CC[N+](CC)(CCCC(C)Nc1c2ccc(Cl)cc2nc2ccc(OC)cc12)Cc1ccc(NC(=O)[C@H](CCCNC(N)=O)NC(=O)[C@@H](NC(=O)CCOCCOCCOCCN2C(=O)C=CC2=O)C(C)C)cc1] |
| A-2 | 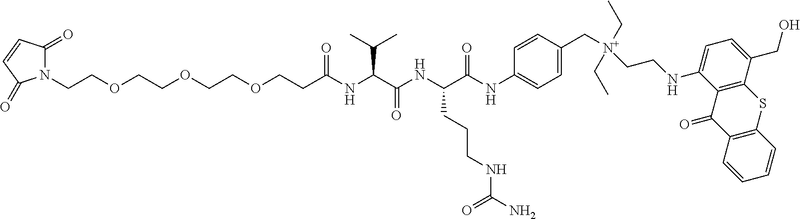 [SMILES: CC[N+](CC)(CCNc1ccc(CO)c2sc3ccccc3c(=O)c12)Cc1ccc(NC(=O)[C@H](CCCNC(N)=O)NC(=O)[C@@H](NC(=O)CCOCCOCCOCCN2C(=O)C=CC2=O)C(C)C)cc1] |
| A-3 | 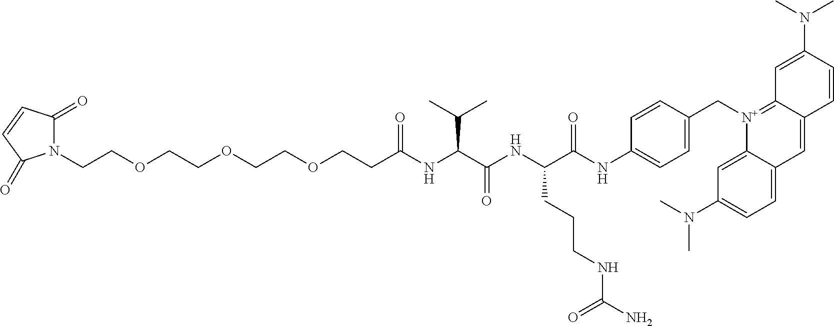 [SMILES: CC(C)[C@H](NC(=O)CCOCCOCCOCCN1C(=O)C=CC1=O)C(=O)N[C@@H](CCCNC(N)=O)C(=O)Nc1ccc(C[n+]2c3cc(N(C)C)ccc3cc3ccc(N(C)C)cc32)cc1] |
| A-4 | 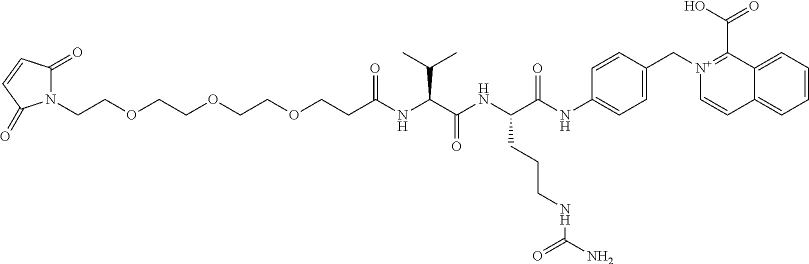 [SMILES: CC(C)[C@H](NC(=O)CCOCCOCCOCCN1C(=O)C=CC1=O)C(=O)N[C@@H](CCCNC(N)=O)C(=O)Nc1ccc(C[n+]2ccc3ccccc3c2C(=O)O)cc1] |
| A-5 | 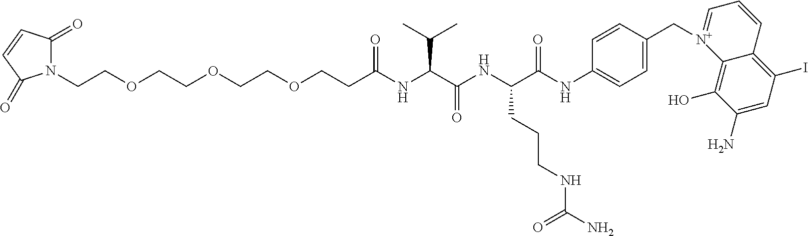 [SMILES: CC(C)[C@H](NC(=O)CCOCCOCCOCCN1C(=O)C=CC1=O)C(=O)N[C@@H](CCCNC(N)=O)C(=O)Nc1ccc(C[n+]2cccc3c(I)cc(N)c(O)c32)cc1] |
| A-6 | 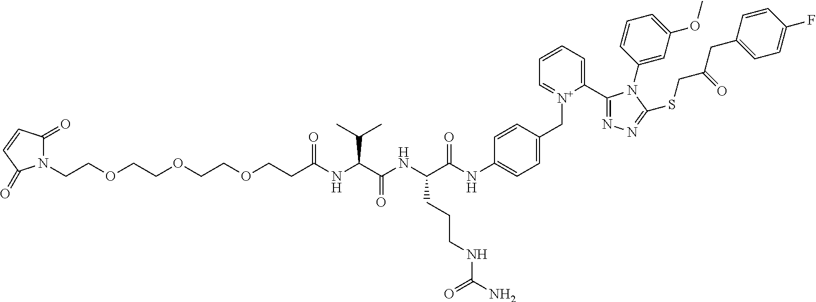 [SMILES: COc1cccc(-n2c(SCC(=O)Cc3ccc(F)cc3)nnc2-c2cccc[n+]2Cc2ccc(NC(=O)[C@H](CCCNC(N)=O)NC(=O)[C@@H](NC(=O)CCOCCOCCOCCN3C(=O)C=CC3=O)C(C)C)cc2)c1] |
| A-7 | 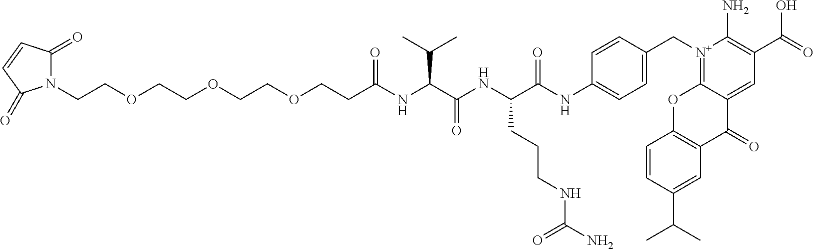 [SMILES: CC(C)c1ccc2oc3c(cc(C(=O)O)c(N)[n+]3Cc3ccc(NC(=O)[C@H](CCCNC(N)=O)NC(=O)[C@@H](NC(=O)CCOCCOCCOCCN4C(=O)C=CC4=O)C(C)C)cc3)c(=O)c2c1] |
| A-8 | 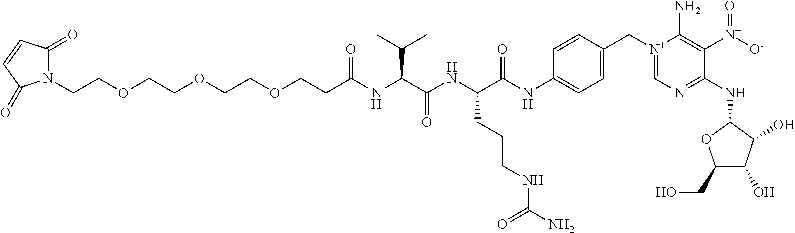 [SMILES: CC(C)[C@H](NC(=O)CCOCCOCCOCCN1C(=O)C=CC1=O)C(=O)N[C@@H](CCCNC(N)=O)C(=O)Nc1ccc(C[n+]2cnc(N[C@H]3O[C@H](CO)[C@@H](O)[C@H]3O)c([N+](=O)[O-])c2N)cc1] |
| A-9 | 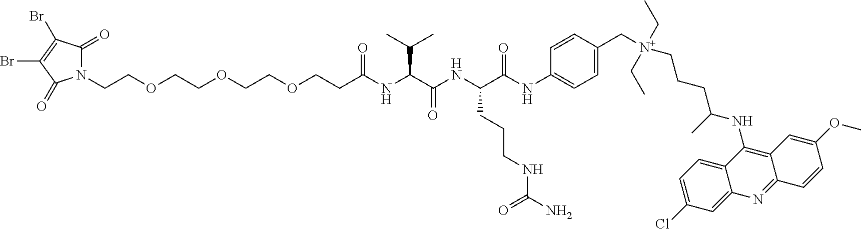 [SMILES: CC[N+](CC)(CCCC(C)Nc1c2ccc(Cl)cc2nc2ccc(OC)cc12)Cc1ccc(NC(=O)[C@H](CCCNC(N)=O)NC(=O)[C@@H](NC(=O)CCOCCOCCOCCN2C(=O)C(Br)=C(Br)C2=O)C(C)C)cc1] |
| A-10 | 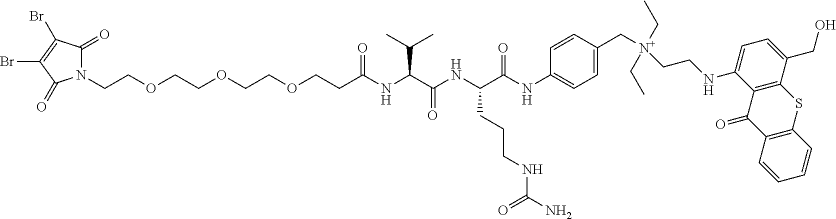 [SMILES: CC[N+](CC)(CCNc1ccc(CO)c2sc3ccccc3c(=O)c12)Cc1ccc(NC(=O)[C@H](CCCNC(N)=O)NC(=O)[C@@H](NC(=O)CCOCCOCCOCCN2C(=O)C(Br)=C(Br)C2=O)C(C)C)cc1] |
| A-11 | 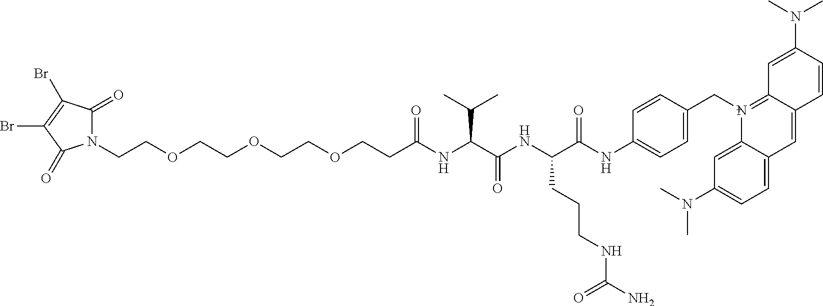 [SMILES: CC(C)[C@H](NC(=O)CCOCCOCCOCCN1C(=O)C(Br)=C(Br)C1=O)C(=O)N[C@@H](CCCNC(N)=O)C(=O)Nc1ccc(C[n+]2c3cc(N(C)C)ccc3cc3ccc(N(C)C)cc32)cc1] |
| A-12 | 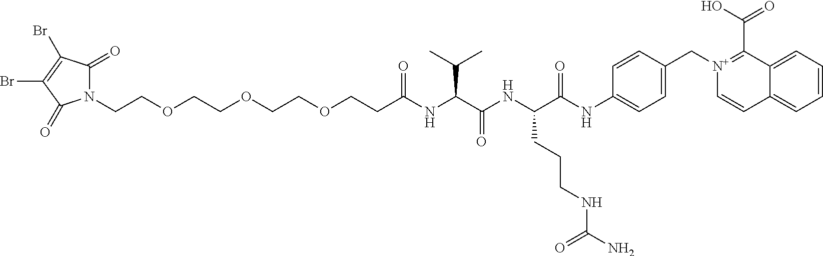 [SMILES: CC(C)[C@H](NC(=O)CCOCCOCCOCCN1C(=O)C(Br)=C(Br)C1=O)C(=O)N[C@@H](CCCNC(N)=O)C(=O)Nc1ccc(C[n+]2ccc3ccccc3c2C(=O)O)cc1] |
| A-13 | 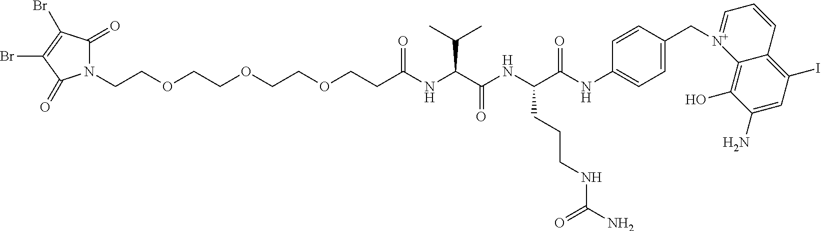 [SMILES: CC(C)[C@H](NC(=O)CCOCCOCCOCCN1C(=O)C(Br)=C(Br)C1=O)C(=O)N[C@@H](CCCNC(N)=O)C(=O)Nc1ccc(C[n+]2cccc3c(I)cc(N)c(O)c32)cc1] |
| A-14 | 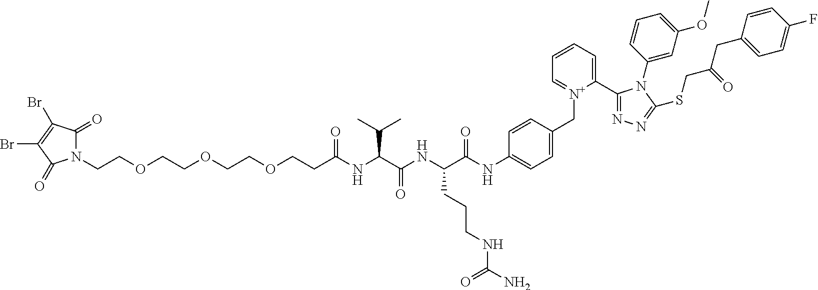 [SMILES: COc1cccc(-n2c(SCC(=O)Cc3ccc(F)cc3)nnc2-c2cccc[n+]2Cc2ccc(NC(=O)[C@H](CCCNC(N)=O)NC(=O)[C@@H](NC(=O)CCOCCOCCOCCN3C(=O)C(Br)=C(Br)C3=O)C(C)C)cc2)c1] |
| A-15 | 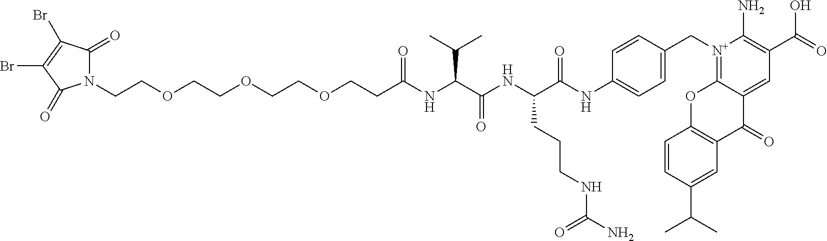 [SMILES: CC(C)c1ccc2oc3c(cc(C(=O)O)c(N)[n+]3Cc3ccc(NC(=O)[C@H](CCCNC(N)=O)NC(=O)[C@@H](NC(=O)CCOCCOCCOCCN4C(=O)C(Br)=C(Br)C4=O)C(C)C)cc3)c(=O)c2c1] |
| A-16 | 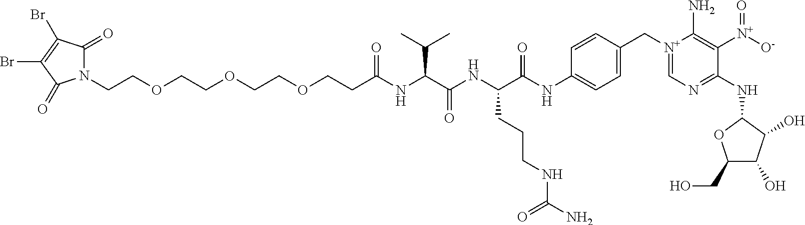 [SMILES: CC(C)[C@H](NC(=O)CCOCCOCCOCCN1C(=O)C(Br)=C(Br)C1=O)C(=O)N[C@@H](CCCNC(N)=O)C(=O)Nc1ccc(C[n+]2cnc(N[C@H]3O[C@H](CO)[C@@H](O)[C@H]3O)c([N+](=O)[O-])c2N)cc1] |
| A-17 | 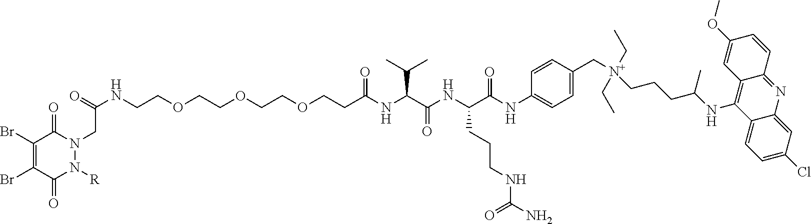 |
| A-18 | 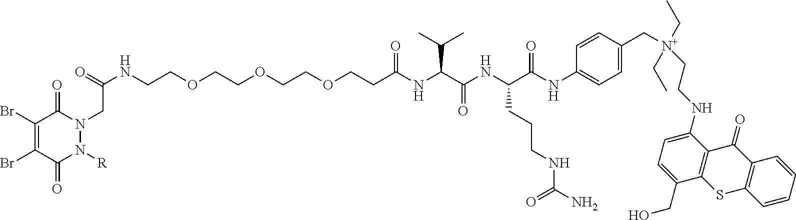 |
| A-19 | 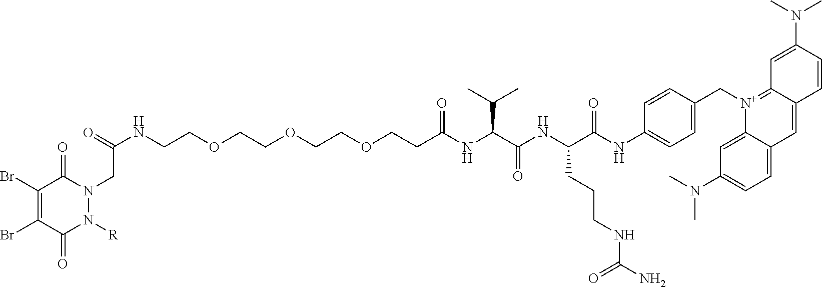 |
| A-20 | 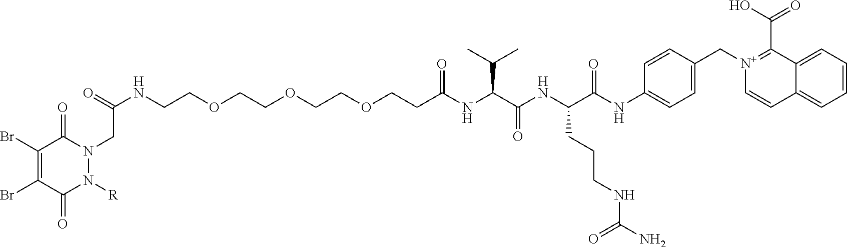 |
| A-21 | 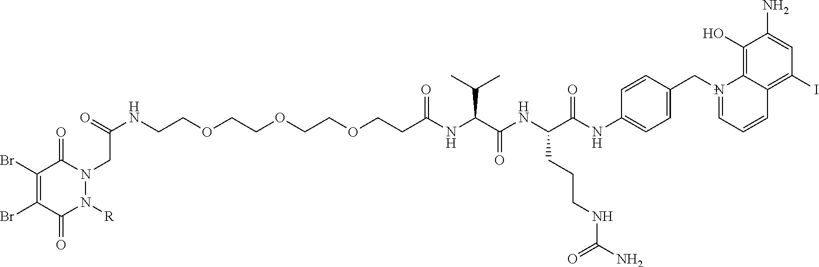 |
| A-22 | 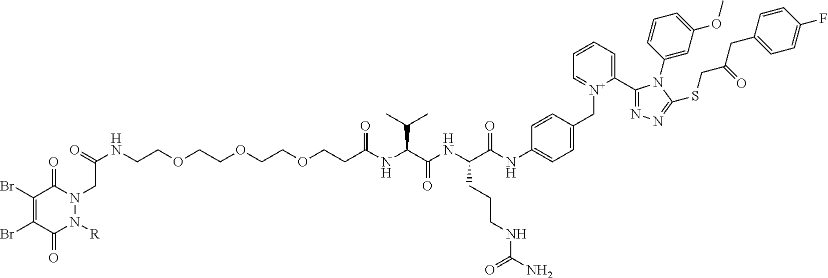 |
| A-23 | 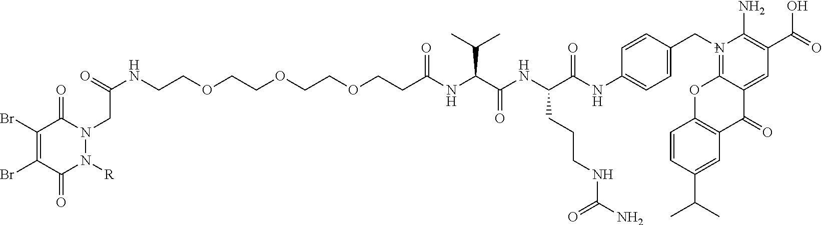 |
| A-24 | 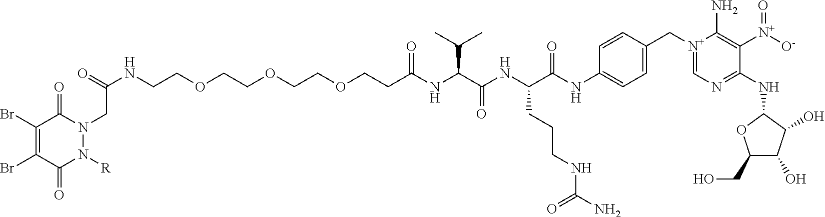 |
| A-25 | 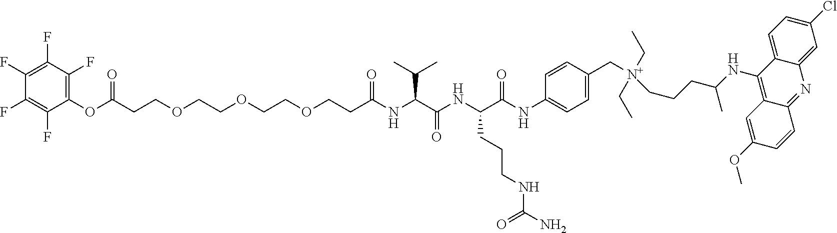 [SMILES: CC[N+](CC)(CCCC(C)Nc1c2ccc(Cl)cc2nc2ccc(OC)cc12)Cc1ccc(NC(=O)[C@H](CCCNC(N)=O)NC(=O)[C@@H](NC(=O)CCOCCOCCOCCC(=O)Oc2c(F)c(F)c(F)c(F)c2F)C(C)C)cc1] |
| A-26 | 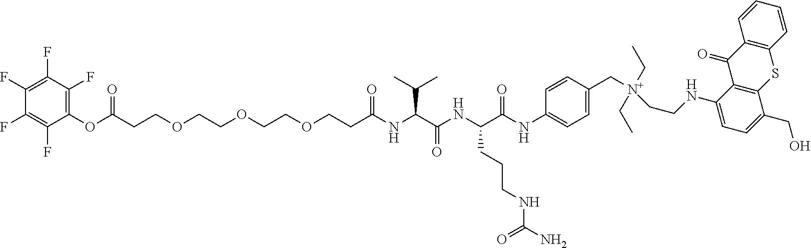 [SMILES: CC[N+](CC)(CCNc1ccc(CO)c2sc3ccccc3c(=O)c12)Cc1ccc(NC(=O)[C@H](CCCNC(N)=O)NC(=O)[C@@H](NC(=O)CCOCCOCCOCCC(=O)Oc2c(F)c(F)c(F)c(F)c2F)C(C)C)cc1] |
| A-27 | 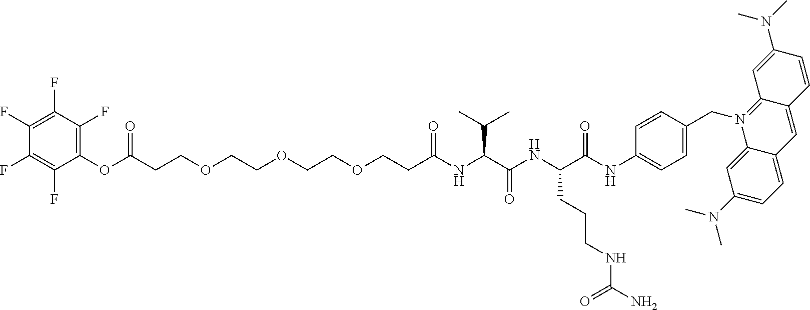 [SMILES: CC(C)[C@H](NC(=O)CCOCCOCCOCCC(=O)Oc1c(F)c(F)c(F)c(F)c1F)C(=O)N[C@@H](CCCNC(N)=O)C(=O)Nc1ccc(C[n+]2c3cc(N(C)C)ccc3cc3ccc(N(C)C)cc32)cc1] |
| A-28 | 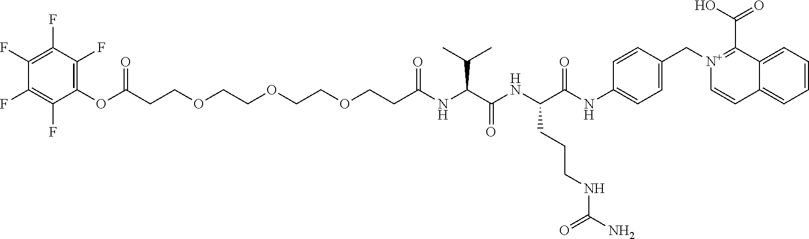 [SMILES: CC(C)[C@H](NC(=O)CCOCCOCCOCCC(=O)Oc1c(F)c(F)c(F)c(F)c1F)C(=O)N[C@@H](CCCNC(N)=O)C(=O)Nc1ccc(C[n+]2ccc3ccccc3c2C(=O)O)cc1] |
| A-29 | 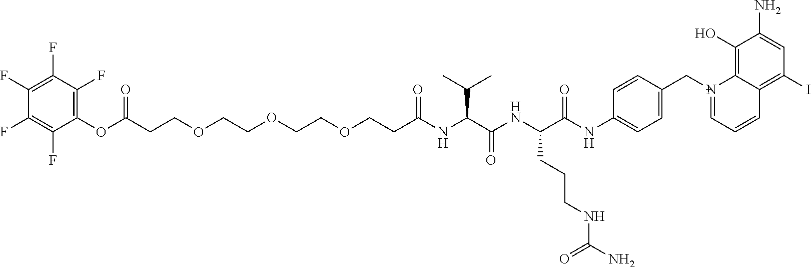 [SMILES: CC(C)[C@H](NC(=O)CCOCCOCCOCCC(=O)Oc1c(F)c(F)c(F)c(F)c1F)C(=O)N[C@@H](CCCNC(N)=O)C(=O)Nc1ccc(C[n+]2cccc3c(I)cc(N)c(O)c32)cc1] |
| A-30 | 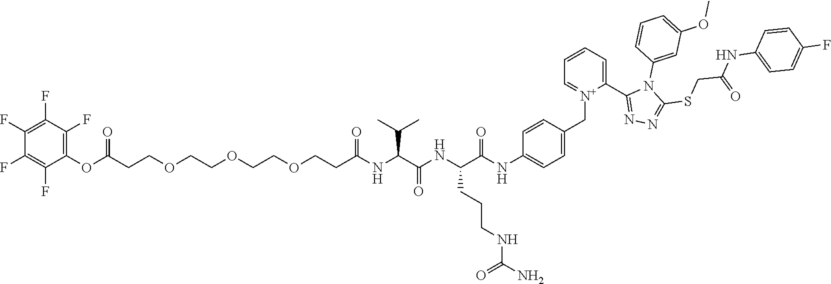 [SMILES: COc1cccc(-n2c(SCC(=O)Nc3ccc(F)cc3)nnc2-c2cccc[n+]2Cc2ccc(NC(=O)[C@H](CCCNC(N)=O)NC(=O)[C@@H](NC(=O)CCOCCOCCOCCC(=O)Oc3c(F)c(F)c(F)c(F)c3F)C(C)C)cc2)c1] |
| A-31 | 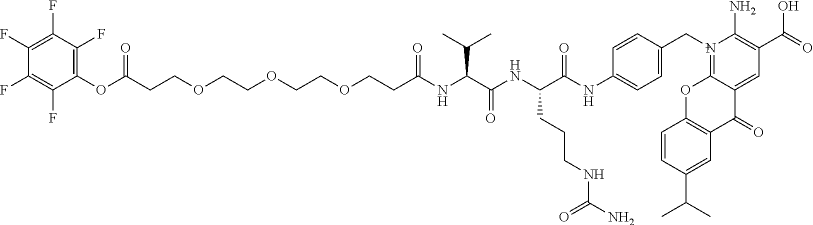 [SMILES: CC(C)c1ccc2oc3c(cc(C(=O)O)c(N)[n+]3Cc3ccc(NC(=O)[C@H](CCCNC(N)=O)NC(=O)[C@@H](NC(=O)CCOCCOCCOCCC(=O)Oc4c(F)c(F)c(F)c(F)c4F)C(C)C)cc3)c(=O)c2c1] |
| A-32 | 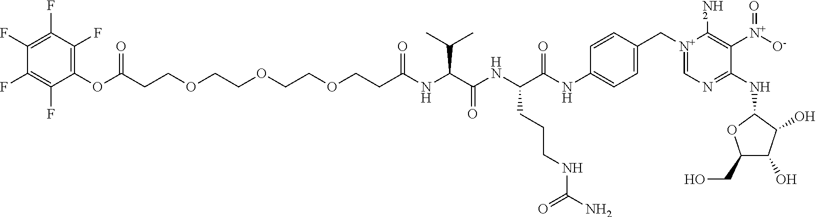 [SMILES: CC(C)[C@H](NC(=O)CCOCCOCCOCCC(=O)Oc1c(F)c(F)c(F)c(F)c1F)C(=O)N[C@@H](CCCNC(N)=O)C(=O)Nc1ccc(C[n+]2cnc(N[C@H]3O[C@H](CO)[C@@H](O)[C@H]3O)c([N+](=O)[O-])c2N)cc1] |
| A-33 | 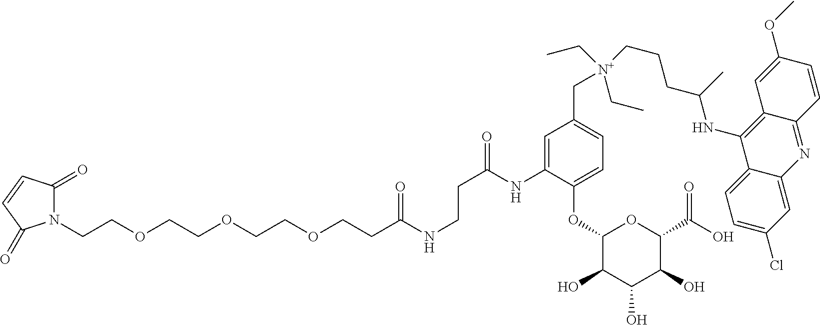 [SMILES: CC[N+](CC)(CCCC(C)Nc1c2ccc(Cl)cc2nc2ccc(OC)cc12)Cc1ccc(O[C@@H]2O[C@H](C(=O)O)[C@@H](O)[C@H](O)[C@H]2O)c(NC(=O)CCNC(=O)CCOCCOCCOCCN2C(=O)C=CC2=O)c1] |
| A-34 | 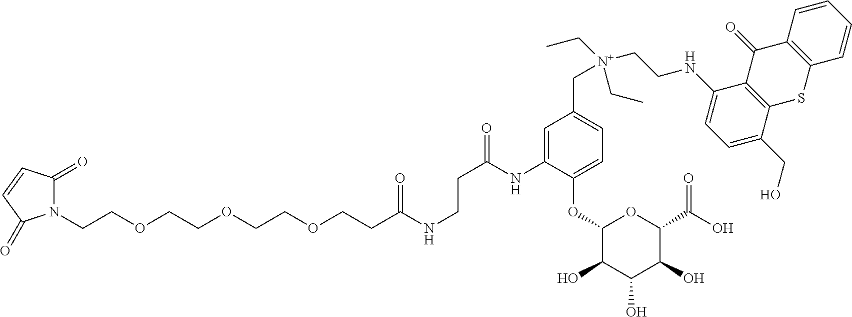 [SMILES: CC[N+](CC)(CCNc1ccc(CO)c2sc3ccccc3c(=O)c12)Cc1ccc(O[C@@H]2O[C@H](C(=O)O)[C@@H](O)[C@H](O)[C@H]2O)c(NC(=O)CCNC(=O)CCOCCOCCOCCN2C(=O)C=CC2=O)c1] |
| A-35 | 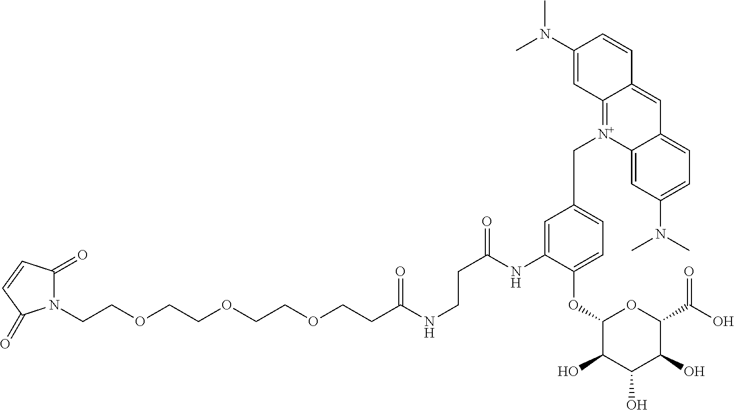 [SMILES: CN(C)c1ccc2cc3ccc(N(C)C)cc3[n+](Cc3ccc(O[C@@H]4O[C@H](C(=O)O)[C@@H](O)[C@H](O)[C@H]4O)c(NC(=O)CCNC(=O)CCOCCOCCOCCN4C(=O)C=CC4=O)c3)c2c1] |
| A-36 | 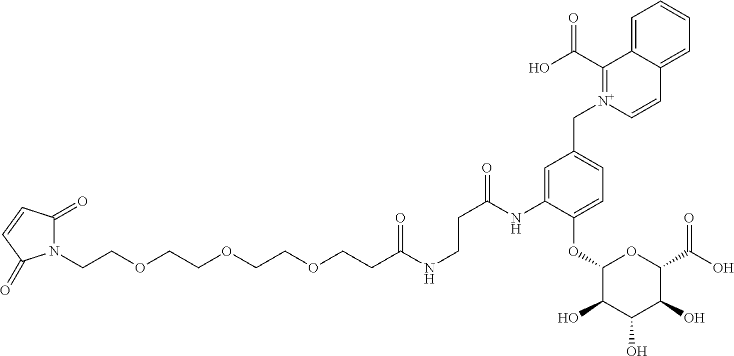 [SMILES: O=C(CCOCCOCCOCCN1C(=O)C=CC1=O)NCCC(=O)Nc1cc(C[n+]2ccc3ccccc3c2C(=O)O)ccc1O[C@@H]1O[C@H](C(=O)O)[C@@H](O)[C@H](O)[C@H]1O] |
| A-37 | 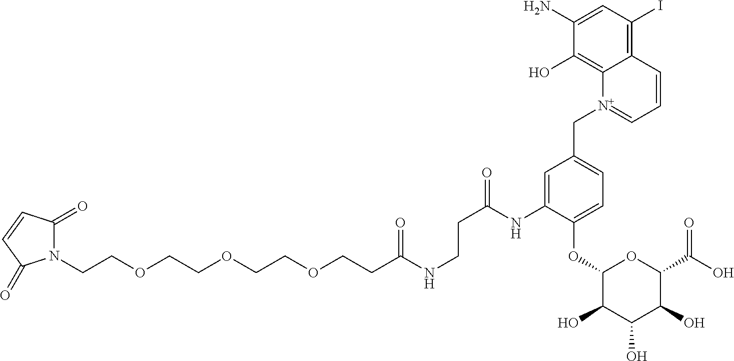 [SMILES: Nc1cc(I)c2ccc[n+](Cc3ccc(O[C@@H]4O[C@H](C(=O)O)[C@@H](O)[C@H](O)[C@H]4O)c(NC(=O)CCNC(=O)CCOCCOCCOCCN4C(=O)C=CC4=O)c3)c2c1O] |
| A-38 | 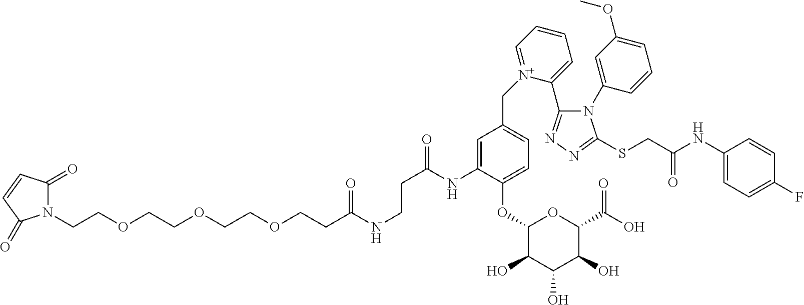 [SMILES: COc1cccc(-n2c(SCC(=O)Nc3ccc(F)cc3)nnc2-c2cccc[n+]2Cc2ccc(O[C@@H]3O[C@H](C(=O)O)[C@@H](O)[C@H](O)[C@H]3O)c(NC(=O)CCNC(=O)CCOCCOCCOCCN3C(=O)C=CC3=O)c2)c1] |
| A-39 | 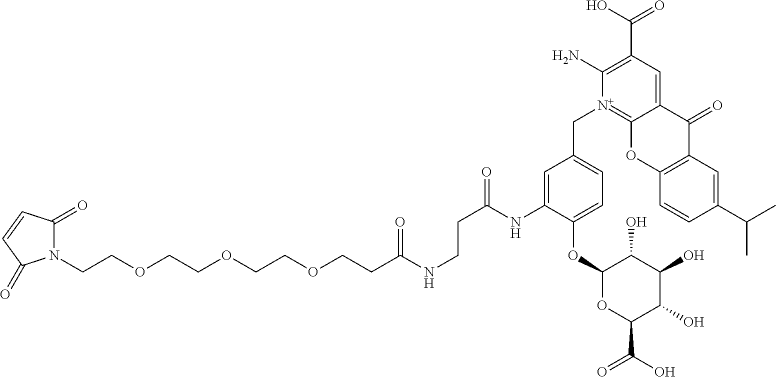 [SMILES: CC(C)c1ccc2oc3c(cc(C(=O)O)c(N)[n+]3Cc3ccc(O[C@@H]4O[C@H](C(=O)O)[C@@H](O)[C@H](O)[C@H]4O)c(NC(=O)CCNC(=O)CCOCCOCCOCCN4C(=O)C=CC4=O)c3)c(=O)c2c1] |
| A-40 | 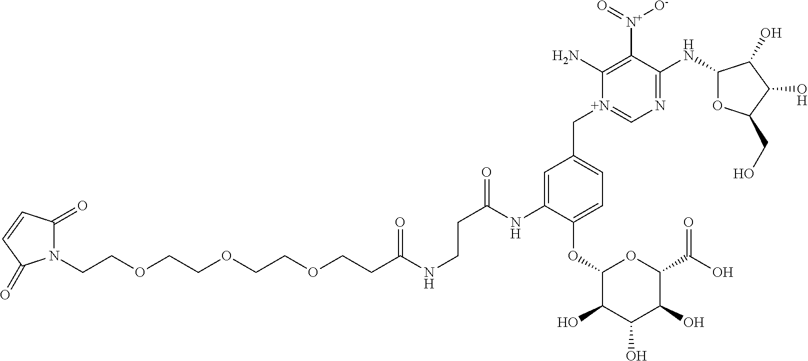 [SMILES: Nc1c([N+](=O)[O-])c(N[C@H]2O[C@H](CO)[C@@H](O)[C@H]2O)nc[n+]1Cc1ccc(O[C@@H]2O[C@H](C(=O)O)[C@@H](O)[C@H](O)[C@H]2O)c(NC(=O)CCNC(=O)CCOCCOCCOCCN2C(=O)C=CC2=O)c1] |
| A-41 | 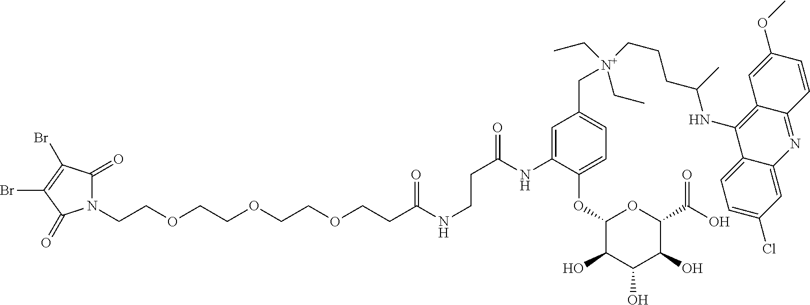 [SMILES: CC[N+](CC)(CCCC(C)Nc1c2ccc(Cl)cc2nc2ccc(OC)cc12)Cc1ccc(O[C@@H]2O[C@H](C(=O)O)[C@@H](O)[C@H](O)[C@H]2O)c(NC(=O)CCNC(=O)CCOCCOCCOCCN2C(=O)C(Br)=C(Br)C2=O)c1] |
| A-42 | 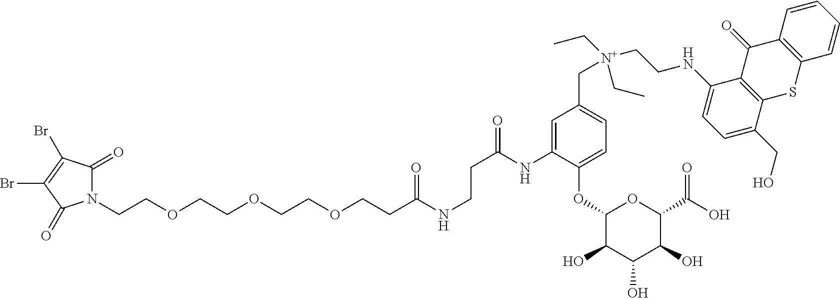 [SMILES: CC[N+](CC)(CCNc1ccc(CO)c2sc3ccccc3c(=O)c12)Cc1ccc(O[C@@H]2O[C@H](C(=O)O)[C@@H](O)[C@H](O)[C@H]2O)c(NC(=O)CCNC(=O)CCOCCOCCOCCN2C(=O)C(Br)=C(Br)C2=O)c1] |
| A-43 | 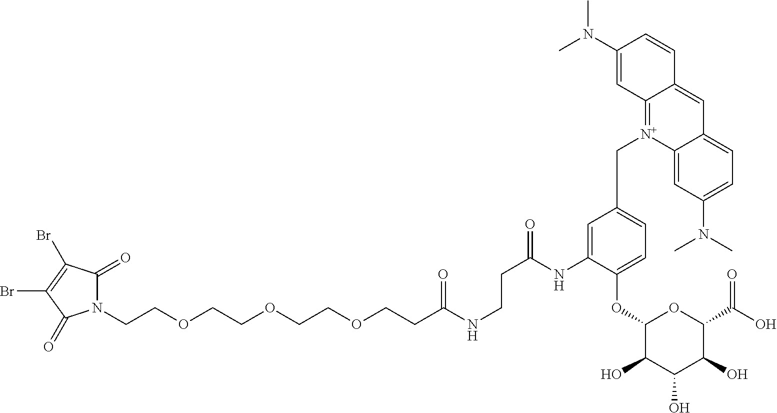 [SMILES: CN(C)c1ccc2cc3ccc(N(C)C)cc3[n+](Cc3ccc(O[C@@H]4O[C@H](C(=O)O)[C@@H](O)[C@H](O)[C@H]4O)c(NC(=O)CCNC(=O)CCOCCOCCOCCN4C(=O)C(Br)=C(Br)C4=O)c3)c2c1] |
| A-44 | 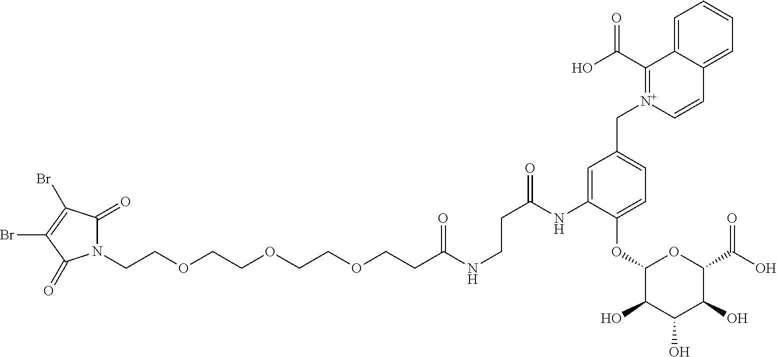 [SMILES: O=C(CCOCCOCCOCCN1C(=O)C(Br)=C(Br)C1=O)NCCC(=O)Nc1cc(C[n+]2ccc3ccccc3c2C(=O)O)ccc1O[C@@H]1O[C@H](C(=O)O)[C@@H](O)[C@H](O)[C@H]1O] |
| A-45 | 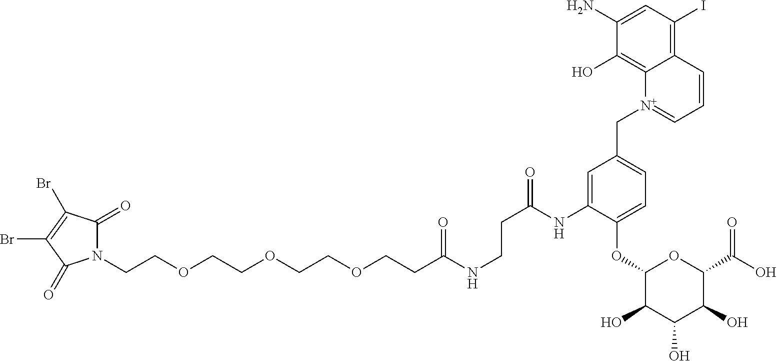 [SMILES: Nc1cc(I)c2ccc[n+](Cc3ccc(O[C@@H]4O[C@H](C(=O)O)[C@@H](O)[C@H](O)[C@H]4O)c(NC(=O)CCNC(=O)CCOCCOCCOCCN4C(=O)C(Br)=C(Br)C4=O)c3)c2c1O] |
| A-46 | 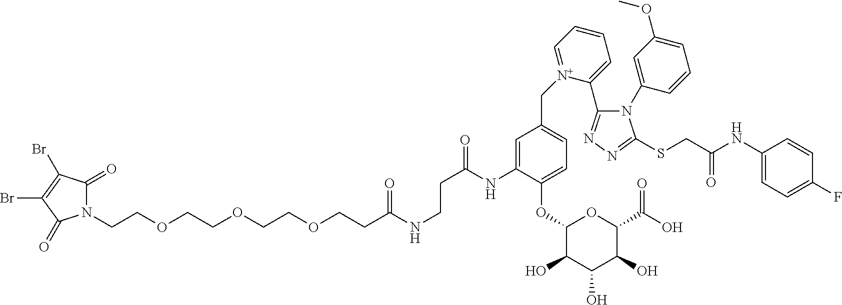 [SMILES: COc1cccc(-n2c(SCC(=O)Nc3ccc(F)cc3)nnc2-c2cccc[n+]2Cc2ccc(O[C@@H]3O[C@H](C(=O)O)[C@@H](O)[C@H](O)[C@H]3O)c(NC(=O)CCNC(=O)CCOCCOCCOCCN3C(=O)C(Br)=C(Br)C3=O)c2)c1] |
| A-47 | 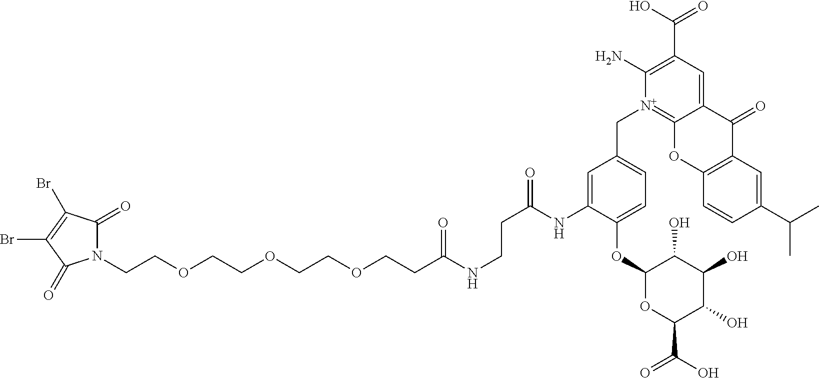 [SMILES: CC(C)c1ccc2oc3c(cc(C(=O)O)c(N)[n+]3Cc3ccc(O[C@@H]4O[C@H](C(=O)O)[C@@H](O)[C@H](O)[C@H]4O)c(NC(=O)CCNC(=O)CCOCCOCCOCCN4C(=O)C(Br)=C(Br)C4=O)c3)c(=O)c2c1] |
| A-48 | 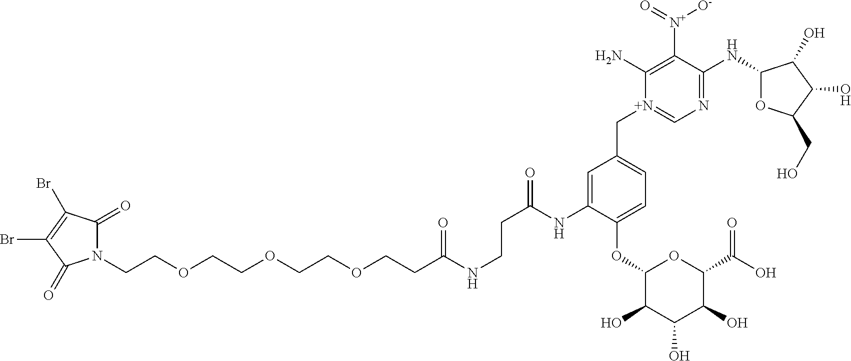 [SMILES: Nc1c([N+](=O)[O-])c(N[C@H]2O[C@H](CO)[C@@H](O)[C@H]2O)nc[n+]1Cc1ccc(O[C@@H]2O[C@H](C(=O)O)[C@@H](O)[C@H](O)[C@H]2O)c(NC(=O)CCNC(=O)CCOCCOCCOCCN2C(=O)C(Br)=C(Br)C2=O)c1] |
| A-49 | 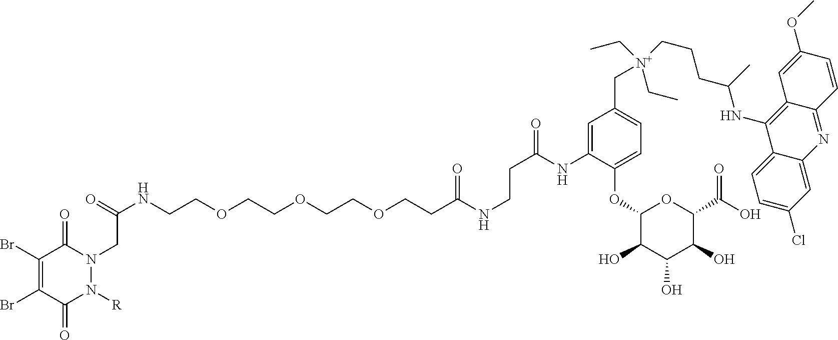 |
| A-50 | 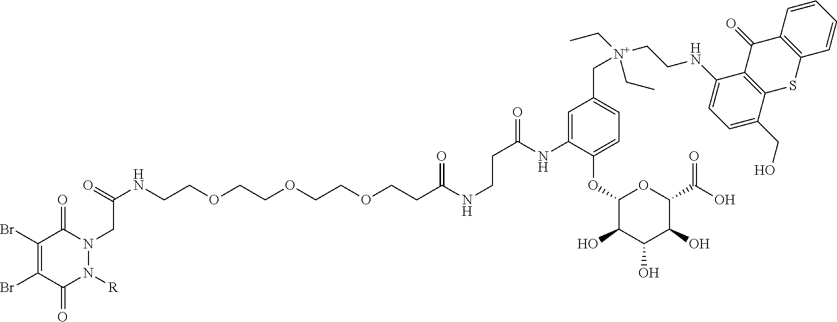 |
| A-51 | 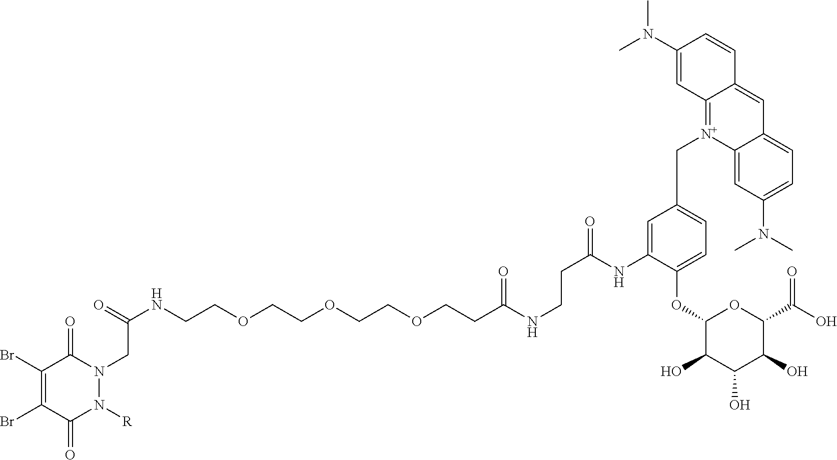 |
| A-52 | 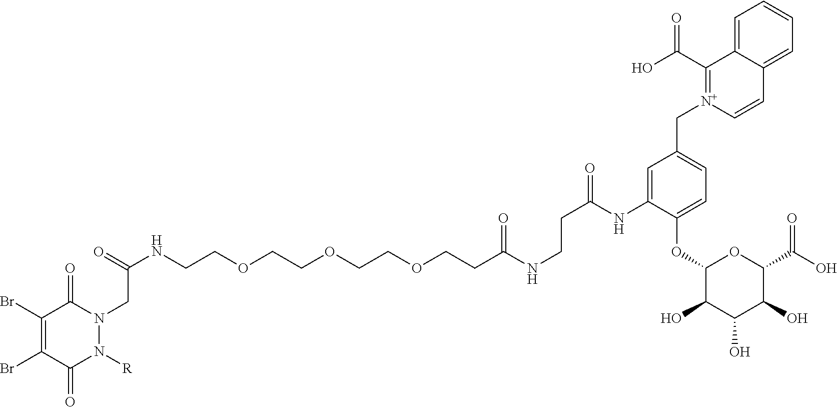 |
| A-53 | 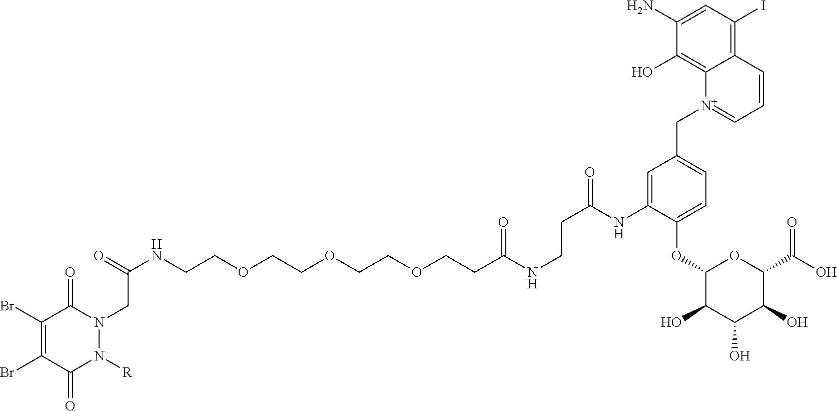 |
| A-54 | 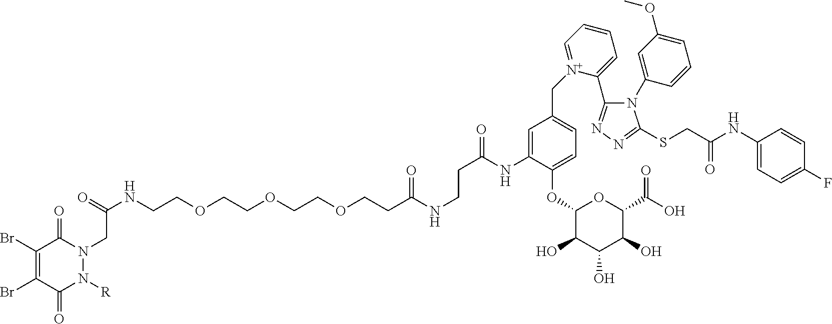 |
| A-55 | 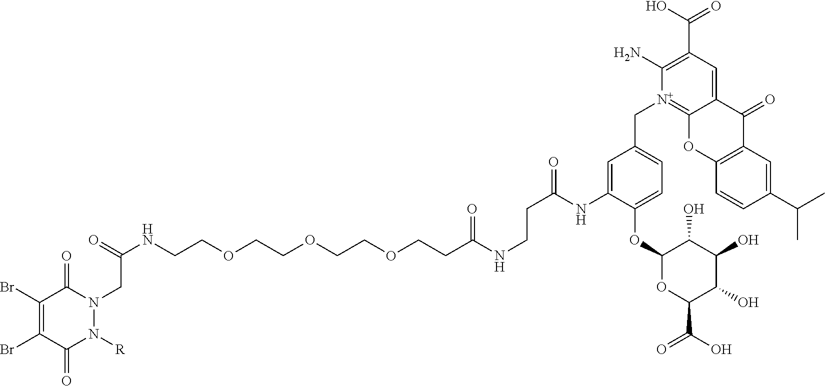 |
| A-56 | 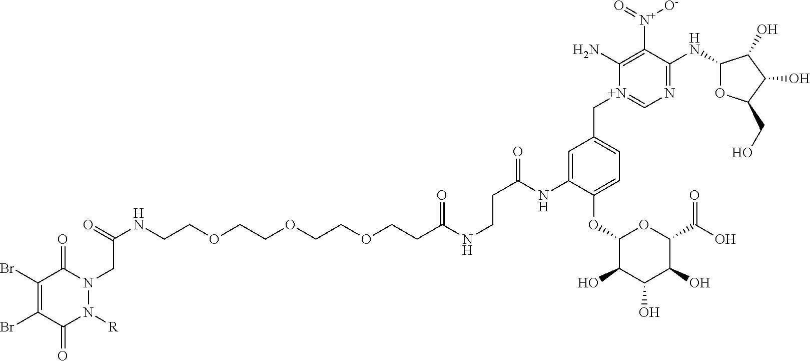 |
| A-57 | 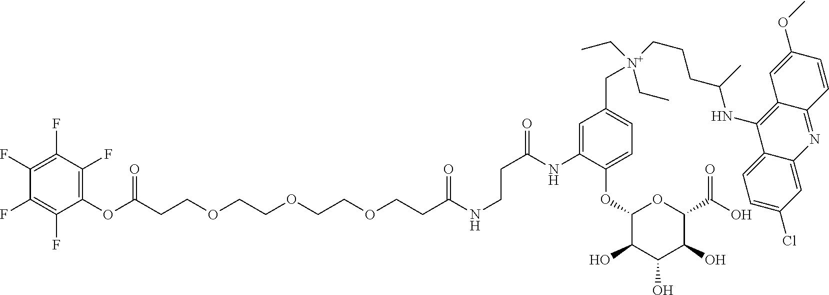 [SMILES: CC[N+](CC)(CCCC(C)Nc1c2ccc(Cl)cc2nc2ccc(OC)cc12)Cc1ccc(O[C@@H]2O[C@H](C(=O)O)[C@@H](O)[C@H](O)[C@H]2O)c(NC(=O)CCNC(=O)CCOCCOCCOCCC(=O)Oc2c(F)c(F)c(F)c(F)c2F)c1] |
| A-58 | 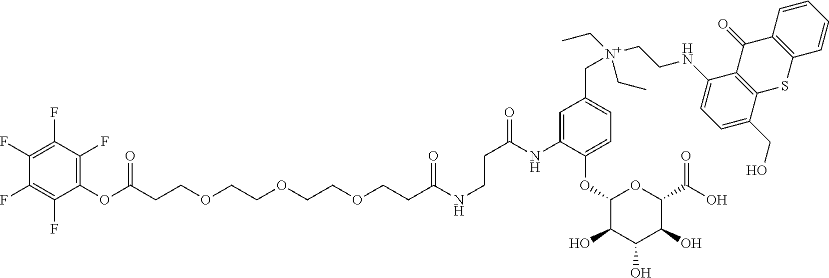 [SMILES: CC[N+](CC)(CCNc1ccc(CO)c2sc3ccccc3c(=O)c12)Cc1ccc(O[C@@H]2O[C@H](C(=O)O)[C@@H](O)[C@H](O)[C@H]2O)c(NC(=O)CCNC(=O)CCOCCOCCOCCC(=O)Oc2c(F)c(F)c(F)c(F)c2F)c1] |
| A-59 | 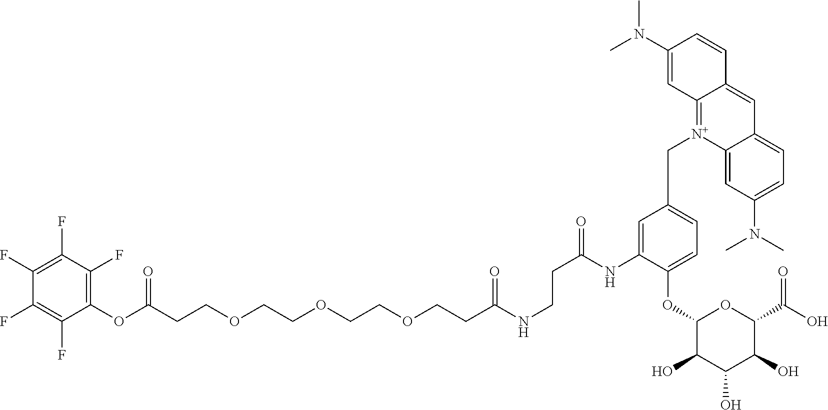 [SMILES: CN(C)c1ccc2cc3ccc(N(C)C)cc3[n+](Cc3ccc(O[C@@H]4O[C@H](C(=O)O)[C@@H](O)[C@H](O)[C@H]4O)c(NC(=O)CCNC(=O)CCOCCOCCOCCC(=O)Oc4c(F)c(F)c(F)c(F)c4F)c3)c2c1] |
| A-60 | 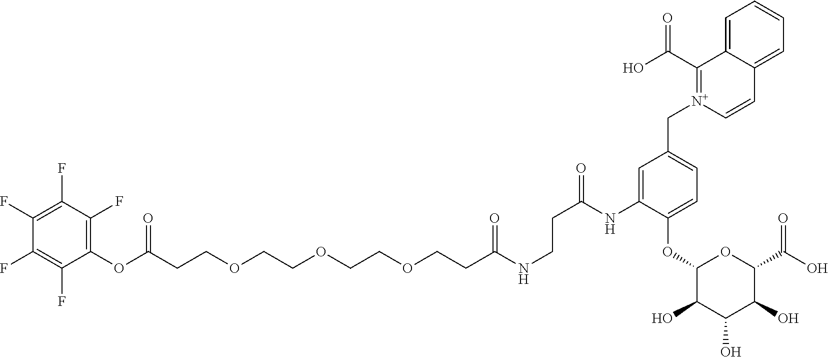 [SMILES: O=C(CCOCCOCCOCCC(=O)Oc1c(F)c(F)c(F)c(F)c1F)NCCC(=O)Nc1cc(C[n+]2ccc3ccccc3c2C(=O)O)ccc1O[C@@H]1O[C@H](C(=O)O)[C@@H](O)[C@H](O)[C@H]1O] |
| A-61 | 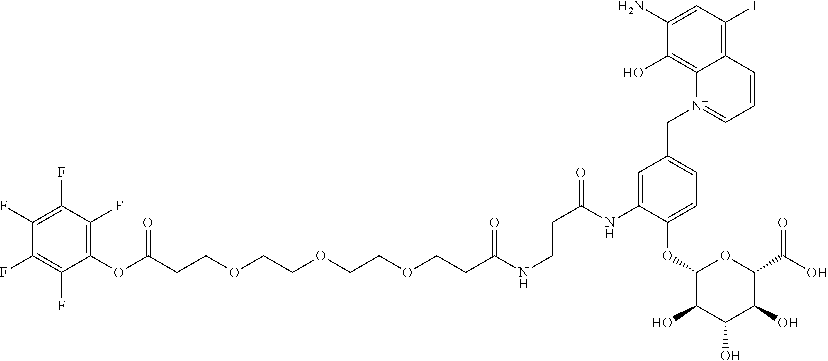 [SMILES: Nc1cc(I)c2ccc[n+](Cc3ccc(O[C@@H]4O[C@H](C(=O)O)[C@@H](O)[C@H](O)[C@H]4O)c(NC(=O)CCNC(=O)CCOCCOCCOCCC(=O)Oc4c(F)c(F)c(F)c(F)c4F)c3)c2c1O] |
| A-62 | 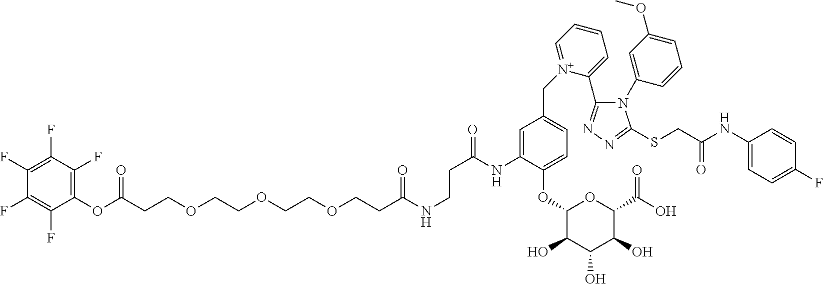 [SMILES: COc1cccc(-n2c(SCC(=O)Nc3ccc(F)cc3)nnc2-c2cccc[n+]2Cc2ccc(O[C@@H]3O[C@H](C(=O)O)[C@@H](O)[C@H](O)[C@H]3O)c(NC(=O)CCNC(=O)CCOCCOCCOCCC(=O)Oc3c(F)c(F)c(F)c(F)c3F)c2)c1] |
| A-63 | 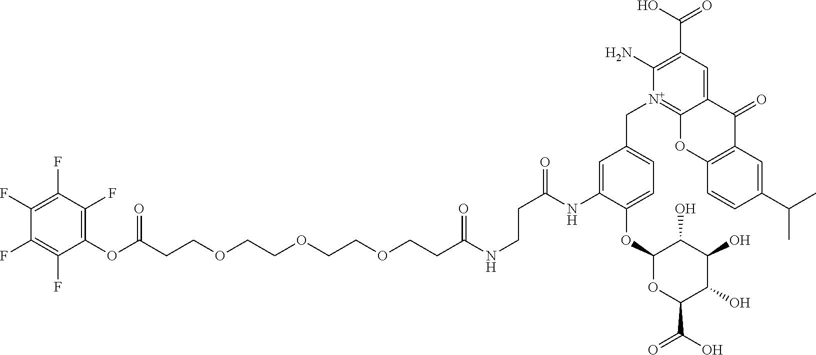 [SMILES: CC(C)c1ccc2oc3c(cc(C(=O)O)c(N)[n+]3Cc3ccc(O[C@@H]4O[C@H](C(=O)O)[C@@H](O)[C@H](O)[C@H]4O)c(NC(=O)CCNC(=O)CCOCCOCCOCCC(=O)Oc4c(F)c(F)c(F)c(F)c4F)c3)c(=O)c2c1] |
| A-64 | 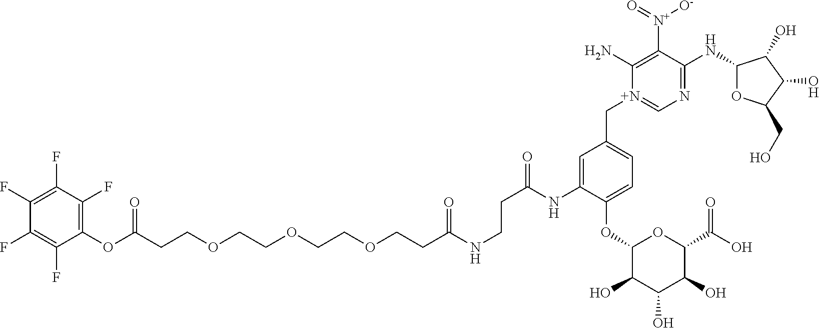 [SMILES: Nc1c([N+](=O)[O-])c(N[C@H]2O[C@H](CO)[C@@H](O)[C@H]2O)nc[n+]1Cc1ccc(O[C@@H]2O[C@H](C(=O)O)[C@@H](O)[C@H](O)[C@H]2O)c(NC(=O)CCNC(=O)CCOCCOCCOCCC(=O)Oc2c(F)c(F)c(F)c(F)c2F)c1] |

In some embodiments, the compound of Formula (I), or a pharmaceutically acceptable salt, solvate, hydrate, isomer, or tautomer thereof, is selected from the group consisting of A-1, A-2, A-3, A-4, A-5, A-6, A-7, A-8, A-9, A-10, A-11, A-12, A-13, A-14, A-15, A-16, A-17, A-18, A-19, A-20, A-21, A-22, A-23, A-24, A-25, A-26, A-27, A-28, A-29, A-30, A-31, A-32, A-33, A-34, A-35, A-36, A-37, A-38, A-39, A-40, A-41, A-42, A-43, A-44, A-45, A-46, A-47, A-48, A-49, A-50, A-51, A-52, A-53, A-54, A-55, A-56, A-57, A-58, A-59, A-60, A-61, A-62, A-63 and A-64.

In one embodiment the present disclosure provides compounds having the structure of Formula (II):

X₁—[X₂—(X₃)ₘ]ₙ(II),

and pharmaceutically acceptable salts, solvates, hydrates, isomers, or tautomers thereof, wherein:
m is 1, 2, 3, 4, 5, 6, 7, 8, 9, or 10;
n is 1, 2, 3, 4, 5, 6, 7, 8, 9, 10, or 11;
X₁ is a biologically active polypeptide or hormone;
X₂ is a linker; and
X₃ is a therapeutic compound.

In another embodiment the present disclosure provides compounds having the structure of Formula (III):

X₁—[X₂—(X₃)ₘ]ₙ (III),

and pharmaceutically acceptable salts, solvates, hydrates, isomers, or tautomers thereof, wherein:
m is 1, 2, 3, 4, 5, 6, 7, 8, 9, or 10;
n is 1, 2, 3, 4, 5, 6, 7, 8, 9, 10, or 11;
X₁ is bispecific antibodies and soluble checkpoint FC's thereof;
X₂ is a linker; and
X₃ is a therapeutic compound.

As shown in Formula (II) and Formula (III), multiple X₃ moieties, the number defined by m, may be attached to X₂, and multiple X₂—(X₃)ₘ moieties, the number defined by n, may be attached to X₁. In other words, multiple therapeutic compounds may be linked to a single linker. And the therapeutic drug-linker compound may be attached to the polypeptide or hormone (X₃) at one or more positions on the polypeptide or hormone. In some embodiments of the compound of Formula (II) or Formula (III), X₂ is bound to X₁ at a Cys residue thereof. In some embodiments of the compound of Formula (II) or Formula (III), X₂ is bound to X₁ at a Lys residue thereof. In some embodiments of the compound of Formula (II) or Formula (III), X₂ is bound to X₁ at two different sites on X₁. In some embodiments of the compound of Formula (II) or Formula (III), X₂ is bound to X₁ at two different Cys residues on X₁. In some embodiments of the compound of Formula (II) or Formula (III), X₂ is bound to X₁ at two different Lys residues on X₁. In some embodiments of the compound of Formula (II) or Formula (III), X₂ is a mixture of X_{2b} and X_{2c}, wherein X_{2b} is a linker that is bound to one Cys residue on X₁ and X_{2c} is a linker that is bound to two Cys residues on X₁, wherein n is a combination of n1 and n2, wherein n1 corresponds to the number of X_{2b} moieties bound to X₁ and n2 corresponds to the number of X_{2c} moieties bound to X₁, and wherein the sum of n1 and n2 is 2, 3, 4, 5, 6, 7, 8, 9, 10, or 11. By forming bonds with two Cys residues on the bioactive polypeptide, the two Cys residues may be rebridged into a disulfide bond, maintaining the structural integrity of the polypeptide and preserving receptor binding and function. The formula of the compound of Formula (II) or Formula (III), when X₂ is a mixture of X_{2b} and X_{2c}, X_{2b} is a linker that is bound to one Cys residue on X₁, X_{2c} is a linker that is bound to two different Cys residues on X₁, n is a combination of n1 and n2, wherein n1 corresponds to the number of X_{2b} moieties bound to X₁ and n2 corresponds to the number of X_{2c} moieties bound to X₁, and wherein the sum of n1 and n2 is 2, 3, 4, 5, 6, 7, 8, 9, 10, or 11, may be illustrated by formula (IV):

[(X₃)ₘ—X_{2b}]ₙ₁—X₁[X_{2c}—(X₃)ₘ]ₙ₂ (IV),

and pharmaceutically acceptable salts, solvates, hydrates, isomers, or tautomers thereof.

The description of n as used in the disclosure is inclusive of n1, n2, and the combination of n1 and n2, with, the sum of n1 and n2 being between 2 and 11.

In some embodiments of the compound of Formula (II) or Formula (III), m is 1. In some embodiments of the compound of Formula (II) or Formula (III), m is 2, 3, 4, 5, 6, 7, or 8. In some embodiments of the compound of Formula (II) or Formula (III), m is 1, 2, or 3. In some embodiments of the compound of Formula (II) or Formula (III), m is 2, 3, 4, or 5. In some embodiments of the compound of Formula (II) or Formula (III), m is 2. In some embodiments of the compound of Formula (II) or Formula (III), m is 3. In some embodiments of the compound of Formula (II) or Formula (III), m is 4. In some embodiments of the compound of Formula (II) or Formula (III), m is 5. In some embodiments of the compound of Formula (II) or Formula (III), m is 6. In some embodiments of the compound of Formula (II) or Formula (III), m is 7. In some embodiments of the compound of Formula (II) or Formula (III), m is 8. In some embodiments of the compound of Formula (II) or Formula (III), m is 9. In some embodiments of the compound of Formula (II) or Formula (III), m is 10. In some embodiments, m is an integer between 1 and 10.

In some embodiments of the compound of Formula (II) or Formula (III), n is 2, 3, 4, 5, 6, 7, 8, or 9. In some embodiments of the compound of Formula (II) or Formula (III), n is 3, 4, 5, 6, 7, or 8. In some embodiments of the compound of Formula (II) or Formula (III), n is 4, 5, 6, or 7. In some embodiments of the compound of Formula (II) or Formula (III), n is 1, 2, 3, 4, 5, or 6. In some embodiments of the compound of Formula (II) or Formula (III), n is 1 or 2. In some embodiments of the compound of Formula (II) or Formula (III), n is 3 or 4. In some embodiments of the compound of Formula (II) or Formula (III), n is 5 or 6. In some embodiments of the compound of Formula (II) or Formula (III), n is 1. In some embodiments of the compound of Formula (II) or Formula (III), n is 2. In some embodiments of the compound of Formula (II) or Formula (III), n is 3. In some embodiments of the compound of Formula (II) or Formula (III), n is 4. In some embodiments of the compound of Formula (II) or Formula (III), n is 5. In some embodiments of the compound of Formula (II) or Formula (III), n is 6. In some embodiments of the compound of Formula (II) or Formula (III), n is 7. In some embodiments of the compound of Formula (II) or Formula (III), n is 8. In some embodiments of the compound of Formula (II) or Formula (III), n is 9. In some embodiments of the compound of Formula (II) or Formula (III), n is 10. In some embodiments of the compound of Formula (II) or Formula (III), n is 11. In some embodiments of the compound of Formula (II) or Formula (III), n is an integer between 2 and 9, an integer between 3 and 8, an integer between 4 and 7, or n can be 5 or 6. In some embodiments of the compound of Formula (II) or Formula (III), n is an integer between 1 and 11.

In some embodiments of the compound of Formula (II) or Formula (III), m=n. In some embodiments of the compound of Formula (II) or Formula (III), n is greater than m. In some embodiments of the compound of Formula (II) or Formula (III), m is 1 and n is 1, 2, 3, 4, 5, 6, 7, 8, 9, 10, or 11. In some embodiments of the compound of Formula (II) or Formula (III), m is 2 and n is 1, 2, 3, 4, 5, 6, 7, 8, 9, 10, or 11. In some embodiments of the compound of Formula (II) or Formula (III), m is 3 and n is 1, 2, 3, 4, 5, 6, 7, 8, 9, 10, or 11. In some embodiments of the compound of Formula (II) or Formula (III), m is 4 and n is 1, 2, 3, 4, 5, 6, 7, 8, 9, 10, or 11. In some embodiments of the compound of Formula (II) or Formula (III), m is 5 and n is 1, 2, 3, 4, 5, 6, 7, 8, 9, 10, or 11. In some embodiments of the compound of Formula (II) or Formula (III), m is 1 and n is 1. In some embodiments of the compound of Formula (II) or Formula (III), m is 1 and n is 2. In some embodiments of the compound of Formula (II) or Formula (III), m is 1 and n is 3. In some embodiments of the compound of Formula (II) or Formula (III), m is 1 and n is 4. In some embodiments of the compound of Formula (II) or Formula (III), m is 1 and n is 5. In some embodiments of the compound of Formula (II) or Formula (III), m is 1 and n is 6. In some embodiments of the compound of Formula (II) or Formula (III), m is 1 and n is 7. In some embodiments of the compound of Formula (II) or Formula (III), m is 1 and n is 8. In some embodiments of the compound of Formula (II) or Formula (III), m is 1 and n is 9. In some embodiments of the compound of Formula (II) or Formula (III), m is 1 and n is 10. In some embodiments of the compound of Formula (II) or Formula (III), m is 1 and n is 11. In some embodiments of the compound of Formula (II) or Formula (III), m is 2 and n is 1. In some embodiments of the compound of Formula (II) or Formula (III), m is 2 and n is 2. In some embodiments of the compound of Formula (II) or Formula (III), m is 2 and n is 3. In some embodiments of the compound of Formula (II) or Formula (III), m is 2 and n is 4. In some embodiments of the compound of Formula (II) or Formula (III), m is 2 and n is 5. In some embodiments of the compound of Formula (II) or Formula (III), m is 2 and n is 6. In some embodiments of the compound of Formula (II) or Formula (III), m is 2 and n is 7.

In some embodiments of the compound of Formula (II) or Formula (III), m is 2 and n is 8. In some embodiments of the compound of Formula (II) or Formula (III), m is 2 and n is 9. In some embodiments of the compound of Formula (II) or Formula (III), m is 2 and n is 10. In some embodiments of the compound of Formula (II) or Formula (III), m is 2 and n is 11. In some embodiments of the compound of Formula (II) or Formula (III), m is 3 and n is 1. In some embodiments of the compound of Formula (II) or Formula (III), m is 3 and n is 2. In some embodiments of the compound of Formula (II) or Formula (III), m is 3 and n is 3. In some embodiments of the compound of Formula (II) or Formula (III), m is 3 and n is 4. In some embodiments of the compound of Formula (II) or Formula (III), m is 3 and n is 5. In some embodiments of the compound of Formula (II) or Formula (III), m is 3 and n is 6. In some embodiments of the compound of Formula (II) or Formula (III), m is 3 and n is 7. In some embodiments of the compound of Formula (II) or Formula (III), m is 3 and n is 8. In some embodiments of the compound of Formula (II) or Formula (III), m is 3 and n is 9. In some embodiments of the compound of Formula (II) or Formula (III), m is 3 and n is 10. In some embodiments of the compound of Formula (II) or Formula (III), m is 3 and n is 11. In some embodiments of the compound of Formula (II) or Formula (III), m is 4 and n is 1. In some embodiments of the compound of Formula (II) or Formula (III), m is 4 and n is 2. In some embodiments of the compound of Formula (II) or Formula (III), m is 4 and n is 3. In some embodiments of the compound of Formula (II) or Formula (III), m is 4 and n is 4. In some embodiments of the compound of Formula (II) or Formula (III), m is 4 and n is 5. In some embodiments of the compound of Formula (II) or Formula (III), m is 4 and n is 6. In some embodiments of the compound of Formula (II) or Formula (III), m is 4 and n is 7. In some embodiments of the compound of Formula (II) or Formula (III), m is 4 and n is 8. In some embodiments of the compound of Formula (II) or Formula (III), m is 4 and n is 9. In some embodiments of the compound of Formula (II) or Formula (III), m is 4 and n is 10. In some embodiments of the compound of Formula (II) or Formula (III), m is 4 and n is 11. In some embodiments of the compound of Formula (II) or Formula (III), m is 5 and n is 1. In some embodiments of the compound of Formula (II) or Formula (III), m is 5 and n is 2. In some embodiments of the compound of Formula (II) or Formula (III), m is 5 and n is 3. In some embodiments of the compound of Formula (II) or Formula (III), m is 5 and n is 4.

In some embodiments of the compound of Formula (II) or Formula (III), m is 5 and n is 5. In some embodiments of the compound of Formula (II) or Formula (III), m is 5 and n is 6. In some embodiments of the compound of Formula (II) or Formula (III), m is 5 and n is 7. In some embodiments of the compound of Formula (II) or Formula (III), m is 5 and n is 8. In some embodiments of the compound of Formula (II) or Formula (III), m is 5 and n is 9. In some embodiments of the compound of Formula (II) or Formula (III), m is 5 and n is 10. In some embodiments of the compound of Formula (II) or Formula (III), m is 5 and n is 11. In some embodiments of the compound of Formula (II) or Formula (III), m is 6 and n is 1. In some embodiments of the compound of Formula (II) or Formula (III), m is 6 and n is 2. In some embodiments of the compound of Formula (II) or Formula (III), m is 6 and n is 3. In some embodiments of the compound of Formula (II) or Formula (III), m is 6 and n is 4. In some embodiments of the compound of Formula (II) or Formula (III), m is 6 and n is 5. In some embodiments of the compound of Formula (II) or Formula (III), m is 6 and n is 6. In some embodiments of the compound of Formula (II) or Formula (III), m is 6 and n is 7. In some embodiments of the compound of Formula (II) or Formula (III), m is 6 and n is 8. In some embodiments of the compound of Formula (II) or Formula (III), m is 6 and n is 9. In some embodiments of the compound of Formula (II) or Formula (III), m is 6 and n is 10. In some embodiments of the compound of Formula (II) or Formula (III), m is 6 and n is 11.

In some embodiments of the compound of Formula (II) or Formula (III), m is 7 and n is 1. In some embodiments of the compound of Formula (II) or Formula (III), m is 7 and n is 2. In some embodiments of the compound of Formula (II) or Formula (III), m is 7 and n is 3. In some embodiments of the compound of Formula (II) or Formula (III), m is 7 and n is 4. In some embodiments of the compound of Formula (II) or Formula (III), m is 7 and n is 5. In some embodiments of the compound of Formula (II) or Formula (III), m is 7 and n is 6. In some embodiments of the compound of Formula (II) or Formula (III), m is 7 and n is 7. In some embodiments of the compound of Formula (II) or Formula (III), m is 7 and n is 8. In some embodiments of the compound of Formula (II) or Formula (III), m is 7 and n is 9. In some embodiments of the compound of Formula (II) or Formula (III), m is 7 and n is 10. In some embodiments of the compound of Formula (II) or Formula (III), m is 7 and n is 11. In some embodiments of the compound of Formula (II) or Formula (III), m is 8 and n is 1. In some embodiments of the compound of Formula (II) or Formula (III), m is 8 and n is 2. In some embodiments of the compound of Formula (II) or Formula (III), m is 8 and n is 3. In some embodiments of the compound of Formula (II) or Formula (III), m is 8 and n is 4. In some embodiments of the compound of Formula (II) or Formula (III), m is 8 and n is 5. In some embodiments of the compound of Formula (II) or Formula (III), m is 8 and n is 6. In some embodiments of the compound of Formula (II) or Formula (III), m is 8 and n is 7. In some embodiments of the compound of Formula (II) or Formula (III), m is 8 and n is 8. In some embodiments of the compound of Formula (II) or Formula (III), m is 8 and n is 9. In some embodiments of the compound of Formula (II) or Formula (III), m is 8 and n is 10. In some embodiments of the compound of Formula (II) or Formula (III), m is 8 and n is 11. In some embodiments of the compound of Formula (II) or Formula (III), m is 9 and n is 1. In some embodiments of the compound of Formula (II) or Formula (III), m is 9 and n is 2. In some embodiments of the compound of Formula (II) or Formula (III), m is 9 and n is 3. In some embodiments of the compound of Formula (II) or Formula (III), m is 9 and n is 4. In some embodiments of the compound of Formula (II) or Formula (III), m is 9 and n is 5. In some embodiments of the compound of Formula (II) or Formula (III), m is 9 and n is 6. In some embodiments of the compound of Formula (II) or Formula (III), m is 9 and n is 7. In some embodiments of the compound of Formula (II) or Formula (III), m is 9 and n is 8. In some embodiments of the compound of Formula (II) or Formula (III), m is 9 and n is 9. In some embodiments of the compound of Formula (II) or Formula (III), m is 9 and n is 10. In some embodiments of the compound of Formula (II) or Formula (III), m is 9 and n is 11. In some embodiments of the compound of Formula (II) or Formula (III), m is 10 and n is 1. In some embodiments of the compound of Formula (II) or Formula (III), m is 10 and n is 2. In some embodiments of the compound of Formula (II) or Formula (III), m is 10 and n is 3. In some embodiments of the compound of Formula (II) or Formula (III), m is 10 and n is 4. In some embodiments of the compound of Formula (II) or Formula (III), m is 10 and n is 5. In some embodiments of the compound of Formula (II) or Formula (III), m is 10 and n is 6. In some embodiments of the compound of Formula (II) or Formula (III), m is 10 and n is 7. In some embodiments of the compound of Formula (II) or Formula (III), m is 10 and n is 8. In some embodiments of the compound of Formula (II) or Formula (III), m is 10 and n is 9. In some embodiments of the compound of Formula (II) or Formula (III), m is 10 and n is 10. In some embodiments of the compound of Formula (II) or Formula (III), m is 10 and n is 11.

In some embodiments of the compound of Formula (II) or Formula (III), X₂ is a mixture of X_{2b} and X_{2c}, wherein X_{2b} is a linker that is bound to one Cys residue on X₁, X_{2c} is a linker that is bound to two Cys residues on X₁, n is a combination of n1 and n2, wherein n1 corresponds to the number of X_{2b} moieties bound to X₁ and n2 corresponds to the number of X_{2c} moieties bound to X₁, and wherein the sum of n1 and n2 is 2, 3, 4, 5, 6, 7, 8, 9, 10, or 11. In some embodiments of the compound of Formula (II) or Formula (III), when X₂ is a mixture of X_{2b} and X_{2c} and n is a combination of n1 and n2, m is 1 and n is 2. In some embodiments of the compound of Formula (II) or Formula (III), when X₂ is a mixture of X_{2b} and X_{2c} and n is a combination of n1 and n2, m is 1 and n is 3. In some embodiments of the compound of Formula (II) or Formula (III), when X₂ is a mixture of X_{2b} and X_{2c} and n is a combination of n1 and n2, m is 1 and n is 4. In some embodiments of the compound of Formula (II) or Formula (III), when X₂ is a mixture of X_{2b} and X_{2c} and n is a mixture of n1 and n2, m is 1 and n is 5. In some embodiments of the compound of Formula (II) or Formula (III), wherein X₂ is a mixture of X_{2b} and X_{2c} and n is a mixture of n1 and n2, m is 1 and n is 6. In some embodiments of the compound of Formula (II) or Formula (III), wherein X₂ is a mixture of X_{2b} and X_{2c} and n is a mixture of n1 and n2, m is 1 and n is 7. In some embodiments of the compound of Formula (II) or Formula (III), wherein X₂ is a mixture of X_{2b} and X_{2c} and n is a mixture of n1 and n2, m is 1 and n is 8. In some embodiments of the compound of Formula (II) or Formula (III), wherein X₂ is a mixture of X_{2b} and X_{2c} and n is a mixture of n1 and n2, m is 1 and n is 9. In some embodiments of the compound of Formula (II) or Formula (III), wherein X₂ is a mixture of X_{2b} and X_{2c} and n is a mixture of n1 and n2, m is 1 and n is 10. In some embodiments of the compound of Formula (II) or Formula (III), wherein X₂ is a mixture of X_{2b} and X_{2c} and n is a mixture of n1 and n2, m is 1 and n is 11. In some embodiments of the compound of Formula (II) or Formula (III), wherein X₂ is a mixture of X_{2b} and X_{2c} and n is a mixture of n1 and n2, wherein X₂ is a mixture of X_{2b} and X_{2c} and n is a mixture of n1 and n2, m is 2 and n is 2. In some embodiments of the compound of Formula (II) or Formula (III), wherein X₂ is a mixture of X_{2b} and X_{2c} and n is a mixture of n1 and n2, m is 2 and n is 3. In some embodiments of the compound of Formula (II) or Formula (III), wherein X₂ is a mixture of X_{2b} and X_{2c} and n is a mixture of n1 and n2, m is 2 and n is 4. In some embodiments of the compound of Formula (II) or Formula (III), wherein X₂ is a mixture of X_{2b} and X_{2c} and n is a mixture of n1 and n2, m is 2 and n is 5. In some embodiments of the compound of Formula (II) or Formula (III), wherein X₂ is a mixture of X_{2b} and X_{2c} and n is a mixture of n1 and n2, m is 2 and n is 6. In some embodiments of the compound of Formula (II) or Formula (III), wherein X₂ is a mixture of X_{2b} and X_{2c} and n is a mixture of n1 and n2, m is 2 and n is 7. In some embodiments of the compound of Formula (II) or Formula (III), wherein X₂ is a mixture of X_{2b} and X_{2c} and n is a mixture of n1 and n2, m is 2 and n is 8. In some embodiments of the compound of Formula (II) or Formula (III), wherein X₂ is a mixture of X_{2b} and X_{2c} and n is a mixture of n1 and n2, m is 2 and n is 9. In some embodiments of the compound of Formula (II) or Formula (III), wherein X₂ is a mixture of X_{2b} and X_{2c} and n is a mixture of n1 and n2, m is 2 and n is 10. In some embodiments of the compound of Formula (II) or Formula (III), wherein X₂ is a mixture of X_{2b} and X_{2c} and n is a mixture of n1 and n2, m is 2 and n is 11.

In some embodiments of the compound of Formula (II) or Formula (III), wherein X₂ is a mixture of X_{2b} and X_{2c} and n is a mixture of n1 and n2, m is 3 and n is 2. In some embodiments of the compound of Formula (II) or Formula (III), wherein X₂ is a mixture of X_{2b} and X_{2c} and n is a mixture of n1 and n2, m is 3 and n is 3. In some embodiments of the compound of Formula (II) or Formula (III), wherein X₂ is a mixture of X_{2b} and X_{2c} and n is a mixture of n1 and n2, m is 3 and n is 4. In some embodiments of the compound of Formula (II) or Formula (III), wherein X₂ is a mixture of X_{2b} and X_{2c} and n is a mixture of n1 and n2, m is 3 and n is 5. In some embodiments of the compound of Formula (II) or Formula (III), wherein X₂ is a mixture of X_{2b} and X_{2c} and n is a mixture of n1 and n2, m is 3 and n is 6. In some embodiments of the compound of Formula (II) or Formula (III), wherein X₂ is a mixture of X_{2b} and X_{2c} and n is a mixture of n1 and n2, m is 3 and n is 7. In some embodiments of the compound of Formula (II) or Formula (III), wherein X₂ is a mixture of X_{2b} and X_{2c} and n is a mixture of n1 and n2, m is 3 and n is 8. In some embodiments of the compound of Formula (II) or Formula (III), wherein X₂ is a mixture of X_{2b} and X_{2c} and n is a mixture of n1 and n2, m is 3 and n is 9. In some embodiments of the compound of Formula (II) or Formula (III), wherein X₂ is a mixture of X_{2b} and X_{2c} and n is a mixture of n1 and n2, m is 3 and n is 10. In some embodiments of the compound of Formula (II) or Formula (III), wherein X₂ is a mixture of X_{2b} and X_{2c} and n is a mixture of n1 and n2, m is 3 and n is 11. In some embodiments of the compound of Formula (II) or Formula (III), wherein X₂ is a mixture of X_{2b} and X_{2c} and n is a mixture of n1 and n2, m is 4 and n is 2. In some embodiments of the compound of Formula (II) or Formula (III), wherein X₂ is a mixture of X_{2b} and X_{2c} and n is a mixture of n1 and n2, m is 4 and n is 3. In some embodiments of the compound of Formula (II) or Formula (III), wherein X₂ is a mixture of X_{2b} and X_{2c} and n is a mixture of n1 and n2, m is 4 and n is 4. In some embodiments of the compound of Formula (II) or Formula (III), wherein X₂ is a mixture of X_{2b} and X_{2c} and n is a mixture of n1 and n2, m is 4 and n is 5. In some embodiments of the compound of Formula (II) or Formula (III), wherein X₂ is a mixture of X_{2b} and X_{2c} and n is a mixture of n1 and n2, m is 4 and n is 6. In some embodiments of the compound of Formula (II) or Formula (III), wherein X₂ is a mixture of X_{2b} and X_{2c} and n is a mixture of n1 and n2, m is 4 and n is 7. In some embodiments of the compound of Formula (II) or Formula (III), wherein X₂ is a mixture of X_{2b} and X_{2c} and n is a mixture of n1 and n2, m is 4 and n is 8. In some embodiments of the compound of Formula (II) or Formula (III), wherein X₂ is a mixture of X_{2b} and X_{2c} and n is a mixture of n1 and n2, m is 4 and n is 9. In some embodiments of the compound of Formula (II) or Formula (III), wherein X₂ is a mixture of X_{2b} and X_{2c} and n is a mixture of n1 and n2, m is 4 and n is 10. In some embodiments of the compound of Formula (II) or Formula (III), wherein X₂ is a mixture of X_{2b} and X_{2c} and n is a mixture of n1 and n2, m is 4 and n is 11. In some embodiments of the compound of Formula (II) or Formula (III), wherein X₂ is a mixture of X_{2b} and X_{2c} and n is a mixture of n1 and n2, m is 5 and n is 2. In some embodiments of the compound of Formula (II) or Formula (III), wherein X₂ is a mixture of X_{2b} and X_{2c} and n is a mixture of n1 and n2, m is 5 and n is 3. In some embodiments of the compound of Formula (II) or Formula (III), wherein X₂ is a mixture of X_{2b} and X_{2c} and n is a mixture of n1 and n2, m is 5 and n is 4. In some embodiments of the compound of Formula (II) or Formula (III), wherein X₂ is a mixture of X_{2b} and X_{2c} and n is a mixture of n1 and n2, m is 5 and n is 5. In some embodiments of the compound of Formula (II) or Formula (III), wherein X₂ is a mixture of X_{2b} and X_{2c} and n is a mixture of n1 and n2, m is 5 and n is 6. In some embodiments of the compound of Formula (II) or Formula (III), wherein X₂ is a mixture of X_{2b} and X_{2c} and n is a mixture of n1 and n2, m is 5 and n is 7. In some embodiments of the compound of Formula (II) or Formula (III), wherein X₂ is a mixture of X_{2b} and X_{2c} and n is a mixture of n1 and n2, m is 5 and n is 8. In some embodiments of the compound of Formula (II) or Formula (III), wherein X₂ is a mixture of X_{2b} and X_{2c} and n is a mixture of n1 and n2, m is 5 and n is 9. In some embodiments of the compound of Formula (II) or Formula (III), wherein X₂ is a mixture of X_{2b} and X₂ and n is a mixture of n1 and n2, m is 5 and n is 10. In some embodiments of the compound of Formula (II) or Formula (III), wherein X₂ is a mixture of X_{2b} and X_{2c} and n is a mixture of n1 and n2, m is 5 and n is 11. In some embodiments of the compound of Formula (II) or Formula (III), wherein X₂ is a mixture of X_{2b} and X_{2c} and n is a mixture of n1 and n2, m is 6 and n is 2. In some embodiments of the compound of Formula (II) or Formula (III), wherein X₂ is a mixture of X_{2b} and X_{2c} and n is a mixture of n1 and n2, m is 6 and n is 3. In some embodiments of the compound of Formula (II) or Formula (III), wherein X₂ is a mixture of X_{2b} and X_{2c} and n is a mixture of n1 and n2, m is 6 and n is 4. In some embodiments of the compound of Formula (II) or Formula (III), wherein X₂ is a mixture of X_{2b} and X_{2c} and n is a mixture of n1 and n2, m is 6 and n is 5. In some embodiments of the compound of Formula (II) or Formula (III), wherein X₂ is a mixture of X_{2b} and X_{2c} and n is a mixture of n1 and n2, m is 6 and n is 6. In some embodiments of the compound of Formula (II) or Formula (III), wherein X₂ is a mixture of X_{2b} and X_{2c} and n is a mixture of n1 and n2, m is 6 and n is 7. In some embodiments of the compound of Formula (II) or Formula (III), wherein X₂ is a mixture of X_{2b} and X_{2c} and n is a mixture of n1 and n2, m is 6 and n is 8. In some embodiments of the compound of Formula (II) or Formula (III), wherein X₂ is a mixture of X_{2b} and X_{2c} and n is a mixture of n1 and n2, m is 6 and n is 9. In some embodiments of the compound of Formula (II) or Formula (III), wherein X₂ is a mixture of X_{2b} and X_{2c} and n is a mixture of n1 and n2, m is 6 and n is 10. In some embodiments of the compound of Formula (II) or Formula (III), wherein X₂ is a mixture of X_{2b} and X_{2c} and n is a mixture of n1 and n2, m is 6 and n is 11. In some embodiments of the compound of Formula (II) or Formula (III), wherein X₂ is a mixture of X_{2b} and X_{2c} and n is a mixture of n1 and n2, m is 7 and n is 2. In some embodiments of the compound of Formula (II) or Formula (III), wherein X₂ is a mixture of X_{2b} and X_{2c} and n is a mixture of n1 and n2, m is 7 and n is 3. In some embodiments of the compound of Formula (II) or Formula (III), wherein X₂ is a mixture of X_{2b} and X_{2c} and n is a mixture of n1 and n2, m is 7 and n is 4. In some embodiments of the compound of Formula (II) or Formula (III), wherein X₂ is a mixture of X_{2b} and X_{2c} and n is a mixture of n1 and n2, m is 7 and n is 5. In some embodiments of the compound of Formula (II) or Formula (III), wherein X₂ is a mixture of X_{2b} and X_{2c} and n is a mixture of n1 and n2, m is 7 and n is 6. In some embodiments of the compound of Formula (II) or Formula (III), wherein X₂ is a mixture of X_{2b} and X_{2c} and n is a mixture of n1 and n2, m is 7 and n is 7. In some embodiments of the compound of Formula (II) or Formula (III), wherein X₂ is a mixture of X_{2b} and X_{2c} and n is a mixture of n1 and n2, m is 7 and n is 8. In some embodiments of the compound of Formula (II) or Formula (III), wherein X₂ is a mixture of X_{2b} and X_{2c} and n is a mixture of n1 and n2, m is 7 and n is 9. In some embodiments of the compound of Formula (II) or Formula (III), wherein X₂ is a mixture of X_{2b} and X_{2c} and n is a mixture of n1 and n2, m is 7 and n is 10. In some embodiments of the compound of Formula (II) or Formula (III), wherein X₂ is a mixture of X_{2b} and X₂ and n is a mixture of n1 and n2, m is 7 and n is 11. In some embodiments of the compound of Formula (II) or Formula (III), wherein X₂ is a mixture of X_{2b} and X_{2c} and n is a mixture of n1 and n2, m is 8 and n is 2. In some embodiments of the compound of Formula (II) or Formula (III), wherein X₂ is a mixture of X_{2b} and X_{2c} and n is a mixture of n1 and n2, m is 8 and n is 3. In some embodiments of the compound of Formula (II) or Formula (III), wherein X₂ is a mixture of X_{2b} and X_{2c} and n is a mixture of n1 and n2, m is 8 and n is 4. In some embodiments of the compound of Formula (II) or Formula (III), wherein X₂ is a mixture of X_{2b} and X_{2c} and n is a mixture of n1 and n2, m is 8 and n is 5. In some embodiments of the compound of Formula (II) or Formula (III), wherein X₂ is a mixture of X_{2b} and X_{2c} and n is a mixture of n1 and n2, m is 8 and n is 6. In some embodiments of the compound of Formula (II) or Formula (III), wherein X₂ is a mixture of X_{2b} and X_{2c} and n is a mixture of n1 and n2, m is 8 and n is 7. In some embodiments of the compound of Formula (II) or Formula (III), wherein X₂ is a mixture of X_{2b} and X_{2c} and n is a mixture of n1 and n2, m is 8 and n is 8. In some embodiments of the compound of Formula (II) or Formula (III), wherein X₂ is a mixture of X_{2b} and X_{2c} and n is a mixture of n1 and n2, m is 8 and n is 9. In some embodiments of the compound of Formula (II) or Formula (III), wherein X₂ is a mixture of X_{2b} and X_{2c} and n is a mixture of n1 and n2, m is 8 and n is 10. In some embodiments of the compound of Formula (II) or Formula (III), wherein X₂ is a mixture of X_{2b} and X_{2c} and n is a mixture of n1 and n2, m is 8 and n is 11. In some embodiments of the compound of Formula (II) or Formula (III), wherein X₂ is a mixture of X_{2b} and X_{2c} and n is a mixture of n1 and n2, m is 9 and n is 2. In some embodiments of the compound of Formula (II) or Formula (III), wherein X₂ is a mixture of X_{2b} and X_{2c} and n is a mixture of n1 and n2, m is 9 and n is 3. In some embodiments of the compound of Formula (II) or Formula (III), wherein X₂ is a mixture of X_{2b} and X_{2c} and n is a mixture of n1 and n2, m is 9 and n is 4. In some embodiments of the compound of Formula (II) or Formula (III), wherein X₂ is a mixture of X_{2b} and X_{2c} and n is a mixture of n1 and n2, m is 9 and n is 5. In some embodiments of the compound of Formula (II) or Formula (III), wherein X₂ is a mixture of X_{2b} and X_{2c} and n is a mixture of n1 and n2, m is 9 and n is 6. In some embodiments of the compound of Formula (II) or Formula (III), wherein X₂ is a mixture of X_{2b} and X_{2c} and n is a mixture of n1 and n2, m is 9 and n is 7. In some embodiments of the compound of Formula (II) or Formula (III), wherein X₂ is a mixture of X_{2b} and X_{2c} and n is a mixture of n1 and n2, m is 9 and n is 8. In some embodiments of the compound of Formula (II) or Formula (III), wherein X₂ is a mixture of X_{2b} and X_{2c} and n is a mixture of n1 and n2, m is 9 and n is 9. In some embodiments of the compound of Formula (II) or Formula (III), wherein X₂ is a mixture of X_{2b} and X_{2c} and n is a mixture of n1 and n2, m is 9 and n is 10. In some embodiments of the compound of Formula (II) or Formula (III), wherein X₂ is a mixture of X_{2b} and X_{2c} and n is a mixture of n1 and n2, m is 9 and n is 11. In some embodiments of the compound of Formula (II) or Formula (III), wherein X₂ is a mixture of X_{2b} and X_{2c} and n is a mixture of n1 and n2, m is 10 and n is 2. In some embodiments of the compound of Formula (II) or Formula (III), wherein X₂ is a mixture of X_{2b} and X_{2c} and n is a mixture of n1 and n2, m is 10 and n is 3. In some embodiments of the compound of Formula (II) or Formula (III), wherein X₂ is a mixture of X_{2b} and X_{2c} and n is a mixture of n1 and n2, m is 10 and n is 4. In some embodiments of the compound of Formula (II) or Formula (III), wherein X₂ is a mixture of X_{2b} and X_{2c} and n is a mixture of n1 and n2, m is 10 and n is 5. In some embodiments of the compound of Formula (II) or Formula (III), wherein X₂ is a mixture of X_{2b} and X_{2c} and n is a mixture of n1 and n2, m is 10 and n is 6. In some embodiments of the compound of Formula (II) or Formula (III), wherein X₂ is a mixture of X_{2b} and X₂ and n is a mixture of n1 and n2, m is 10 and n is 7. In some embodiments of the compound of Formula (II) or Formula (III), wherein X₂ is a mixture of X_{2b} and X_{2c} and n is a mixture of n1 and n2, m is 10 and n is 8. In some embodiments of the compound of Formula (II) or Formula (III), wherein X₂ is a mixture of X_{2b} and X_{2c} and n is a mixture of n1 and n2, m is 10 and n is 9. In some embodiments of the compound of Formula (II) or Formula (III), wherein X₂ is a mixture of X_{2b} and X_{2c} and n is a mixture of n1 and n2, m is 10 and n is 10. In some embodiments of the compound of Formula (II) or Formula (III), wherein X₂ is a mixture of X_{2b} and X_{2c} and n is a mixture of n1 and n2, m is 10 and n is 11. In some embodiments of the compound of Formula (II) or Formula (III), wherein X₂ is a mixture of X_{2b} and X_{2c} and n is a mixture of n1 and n2, n1 is 2 or 3 and n2 is 1. In some embodiments of the compound of Formula (II) or Formula (III), wherein X₂ is a mixture of X_{2b} and X_{2c} and n is a mixture of n1 and n2, n1 is 2 and n2 is 1. In some embodiments of the compound of Formula (II) or Formula (III), wherein X₂ is a mixture of X_{2b} and X_{2c} and n is a mixture of n1 and n2, n1 is 3 and n2 is 1. In some embodiments of the compound of Formula (II) or Formula (III), wherein X₂ is a mixture of X_{2b} and X_{2c} and n is a mixture of n1 and n2, n1 is 1, 2, 3, 4, or 5 and n2 is 1, 2 or 3. In some embodiments of the compound of Formula (II) or Formula (III), wherein X₂ is a mixture of X_{2b} and X_{2c} and n is a mixture of n1 and n2, n1 is 1, 2, 3, 4, or 5 and n2 is 1. In some embodiments of the compound of Formula (II) or Formula (III), wherein X₂ is a mixture of X_{2b} and X_{2c} and n is a mixture of n1 and n2, n1 is 1, 2, 3, 4, or 5 and n2 is 2. In some embodiments of the compound of Formula (II) or Formula (III), wherein X₂ is a mixture of X_{2b} and X_{2c} and n is a mixture of n1 and n2, n1 is 1, 2, 3, 4, or 5 and n2 is 3. In some embodiments of the compound of Formula (II) or Formula (III), wherein X₂ is a mixture of X_{2b} and X_{2c} and n is a mixture of n1 and n2, n1 is 1 and n2 is 1, 2 or 3. In some embodiments of the compound of Formula (II) or Formula (III), wherein X₂ is a mixture of X_{2b} and X_{2c} and n is a mixture of n1 and n2, n1 is 2 and n2 is 1, 2 or 3. In some embodiments of the compound of Formula (II) or Formula (III), wherein X₂ is a mixture of X_{2b} and X_{2c} and n is a mixture of n1 and n2, n1 is 3 and n2 is 1, 2 or 3. In some embodiments of the compound of Formula (II) or Formula (III), wherein X₂ is a mixture of X_{2b} and X_{2c} and n is a mixture of n1 and n2, n1 is 4 and n2 is 1, 2 or 3. In some embodiments of the compound of Formula (II) or Formula (III), wherein X₂ is a mixture of X_{2b} and X_{2c} and n is a mixture of n1 and n2, n1 is 5 and n2 is 1, 2 or 3. In some embodiments of the compound of Formula (II) or Formula (III), wherein X₂ is a mixture of X_{2b} and X_{2c} and n is a mixture of n1 and n2, n1=n2. In some embodiments, each X₂ bound to X₁ is an X_{2b} moiety. In some embodiments, each X₂ bound to X₁ is an X_{2c} moiety.

In some embodiments of the compound of Formula (II) or Formula (III), the molar ratio of X₃ to X₁ is between 1:1 and 110:1. In some embodiments of the compound of Formula (II) or Formula (III), the molar ratio of X₃ to X₁ is between 1:1 and 50:1. In some embodiments of the compound of Formula (II) or Formula (III), the molar ratio of X₃ to X₁ is between 1:1 and 10:1. In some embodiments of the compound of Formula (II) or Formula (III), the molar ratio of X₃ to X₁ is between 1:1 and 5:1. In some embodiments of the compound of Formula (II) or Formula (III), the molar ratio of X₃ to X₁ is between 10:1 and 50:1. In some embodiments of the compound of Formula (II) or Formula (III), the molar ratio of X₃ to X₁ is between 50:1 and 110:1. In some embodiments of the compound of Formula (II) or Formula (III), the molar ratio of X₃ to X₁ is 1:1. In some embodiments of the compound of Formula (II) or Formula (III), the molar ratio of X₃ to X₁ is 2:1. In some embodiments of the compound of Formula (II) or Formula (III), the molar ratio of X₃ to X₁ is 3:1. In some embodiments of the compound of Formula (II) or Formula (III), the molar ratio of X₃ to X₁ is 4:1. In some embodiments of the compound of Formula (II) or Formula (III), the molar ratio of X₃ to X₁ is 5:1. In some embodiments of the compound of Formula (II) or Formula (III), the molar ratio of X₃ to X₁ is 6:1. In some embodiments of the compound of Formula (II) or Formula (III), the molar ratio of X₃ to X₁ is 7:1. In some embodiments of the compound of Formula (II) or Formula (III), the molar ratio of X₃ to X₁ is 8:1. In some embodiments of the compound of Formula (II) or Formula (III), the molar ratio of X₃ to X₁ is 9:1. In some embodiments of the compound of Formula (II) or Formula (III), the molar ratio of X₃ to X₁ is 10:1. In some embodiments of the compound of Formula (II) or Formula (III), the molar ratio of X₃ to X₁ is 11:1. In some embodiments of the compound of Formula (II) or Formula (III), the molar ratio of X₃ to X₁ is 12:1. In some embodiments of the compound of Formula (II) or Formula (III), the molar ratio of X₃ to X₁ is 13:1. In some embodiments of the compound of Formula (II) or Formula (III), the molar ratio of X₃ to X₁ is 14:1. In some embodiments of the compound of Formula (II) or Formula (III), the molar ratio of X₃ to X₁ is 15:1. In some embodiments of the compound of Formula (II) or Formula (III), the molar ratio of X₃ to X₁ is 16:1. In some embodiments of the compound of Formula (II) or Formula (III), the molar ratio of X₃ to X₁ is 17:1. In some embodiments of the compound of Formula (II) or Formula (III), the molar ratio of X₃ to X₁ is 18:1. In some embodiments of the compound of Formula (II) or Formula (III), the molar ratio of X₃ to X₁ is 19:1. In some embodiments of the compound of Formula (II) or Formula (III), the molar ratio of X₃ to X₁ is 20:1.

X₁ is checkpoint ligand targeting recombinant proteins and antibodies. PD1-ECD-FC was designed as the extracellular domain of the Human PD1 gene product fused to the Human IgG4 isotype heavy chain constant region gene product. TIM3-ECD-FC was designed as the extracellular domain of the Human TIM3 gene product fused to the Human IgG4 isotype heavy chain constant region gene product. BTLA-ECD-FC was designed as the extracellular domain of the Human BTLA gene product fused to the Human IgG4 isotype heavy chain constant region gene product. CTLA-4-ECD-FC was designed as the extracellular domain of the Human CTLA-4 gene product fused to the Human IgG4 isotype heavy chain constant region gene product. GITR-ECD-FC was designed as the extracellular domain of the Human GITR gene product fused to the Human IgG4 isotype heavy chain constant region gene product. Anti-Human PDL1-HVEM bispecific antibody is based on the primary published sequence for the Atezolimumab anti-Human PDL1 antibody (Genentech/Roche) and the anti-Human HVEM antibody (4C Biomed) in a bispecific single chain format consisting only of the variable heavy and light chains from both antibodies.

In some embodiments, the bispecific antibodies and soluble checkpoint FC's of the disclosure have at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% amino acid sequence identity to an amino acid sequence of Table 4. In some embodiments bispecific antibodies and soluble checkpoint FC's of the disclosure have at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% amino acid sequence identity.

TABLE 4
Amino Acid Sequences of Proteins and Polypeptides

| SEQ ID & Name | Amino Acid Sequence |
|---|---|
| PD1-ECD-FC | FLDSPDRPWNPPTFSPALLVVTEGDNATFTCSFSNTSESFVLNWYRMSPSNQTDKLAAFPEDRSQPGQDCRF |
| | RVTQLPNGRDFHMSVVRARRNDSGTYLCGAISLAPKAQIKESLRAELRVTERRAEVPTAHPSPSPRPAGQFQ |
| | TLVGSAM P P C P S C P A P E F L G G P S V F L F P P K P K D T L M I S R |
| | T P E V T C V V V D V S Q E D P E V Q F N W Y V D G V E V H N A K T K P |
| | R E E Q F N S T Y R V V S V L T V L H Q D W L N G K E Y K C K V S N K G |
| | L P S S I E K T I S K A K G Q P R E P Q V Y T L P P S Q E E M T K N Q V |
| | S L T C L V K G F Y P S D I A V E W E S N G Q P E N N Y K T T P P V L D |
| | S D G S F F L Y S R L T V D K S R W Q E G N V F S C S V M H E A L H N H |
| | Y T Q K S L S L S L G K |
| TIM3-ECD-FC | QSALTQPRSVSGSPGQSVTISCTGTSSDVGGYNYVSWYQQHPGKAPKLMIYDVSKRPSGVPDRFSGSKSGNT |
| | ASLTISLQAEDEADYYCSSYADSVVFGGGTKVTVLGQPKAAPSVTLFPPSSEELQANKATLVCLISDFYPGA |
| | VTVAWKADSSPVKAGVETTTPSKQSNNKYAASSYLSLPTEQWKSHKSYSCQVTHEGSTVEKTVAPTECSGSA |
| | M P P C P S C P A P E F L G G P S V F L F P P K P K D T L M I S R T P E |
| | V T C V V V D V S Q E D P E V Q F N W Y V D G V E V H N A K T K P R E E |
| | Q F N S T Y R V V S V L T V L H Q D W L N G K E Y K C K V S N K G L P S |
| | S I E K T I S K A K G Q P R E P Q V Y T L P P S Q E E M T K N Q V S L T |
| | C L V K G F Y P S D I A V E W E S N G Q P E N N Y K T T P P V L D S D G |
| | S F F L Y S R L T V D K S R W Q E G N V F S C S V M H E A L H N H Y T Q |
| | K S L S L S L G K |
| BTLA-ECD-FC | KESCDVQLYIKRQSEHSILAGDPFELECPVKYCANRPHVTWCKLNGTTCVKLEDRQTSWKEEKNISFFILHF |
| | EPVLPNDNGSYRCSANFQSNLIESHSTTLYVTDVKSASERPSKDEMASRPWLLYRGSAM P P C P S C |
| | P A P E F L G G P S V F L F P P K P K D T L M I S R T P E V T C V V V D |
| | V S Q E D P E V Q F N Y V D G V E V H N A K T K P R E E Q F N S T Y R V |
| | V S V L T V L H Q D W L N G K E Y K C K V S N K G L P S S I E K T I S K |
| | A K G Q P R E P Q V Y T L P P S Q E E M T K N Q V S L T C L V K G F Y P |
| | S D I A V E W E S N G Q P E N N Y K T T P P V L D S D G S F F L Y S R L |
| | T V D K S R W Q E G N V F S C S V M H E A L H N H Y T Q K S L S L S L G |
| | K |
| CTLA-4-ECD-FC | KAMHVAQPAVVLASSRGIASFVCEYASPGKATEVRVTVLRQADSQVTEVCAATYMMGNELTFLDDSICTGTS |
| | SGNQVNLTIQGLRAMDTGLYICKVELMYPPPYYLGIGNGTQIYVIDPEPCPDSDGSAM P P C P S C P |
| | A P E F L G G P S V F L F P P K P K D T L M I S R T P E V T C V V V D V |
| | S Q E D P E V Q F N W Y V D G V E V H N A K T K P R E E Q F N S T Y R V |
| | V S V L T V L H Q D W L N G K E Y K C K V S N K G L P S S I E K T I S K |
| | A K G Q P R E P Q V Y T L P P S Q E E M T K N Q V S L T C L V K G F Y P |
| | S D I A V E W E S N G Q P E N N Y K T T P P V L D S D G S F F L Y S R L |
| | T V D K S R W Q E G N V F S C S V M H E A L H N H Y T Q K S L S L S L G |
| | K |
| GITR-ECD-FC | QRPTGGPGCGPGRLLLGTGTDARCCRVHTTRCCRDYPGEECCSEWDCMCVQPEFHCGDPCCTTCRHHPCPPG |
| | QGVQSQGKTFSFGFQCIDCASGTFSGGHEGHCKPWTDCTQFGFLTVFPGNKTHNAVCVPGSPPAEP GSAM |
| | P P C P S C P A P E F L G G P S V F L F P P K P K D T L M I S R T P E V |
| | T C V V V D V S Q E D P E V Q F N W Y V D G V E V H N A K T K P R E E Q |
| | F N S T Y R V V S V L T V L H Q D W L N G K E Y K C K V S N K G L P S S |
| | I E K T I S K A K G Q P R E P Q V Y T L P P S Q E E M T K N Q V S L T C |
| | L V K G F Y P S D I A V E W E S N G Q P E N N Y K T T P P V L D S D G S |
| | F F L Y S R L T V D K S R W Q E G N V F S C S V M H E A L H N H Y T Q K |
| | S L S L S L G K |
| Anti-Human PDL1- | EVQLVESGGGLVQPGGSLRLSCAASGFTFSDSWIHWVRQAPGKGLEWVAWISPYGGSTYYADSVKGRFTISA |
| HVEM bispecific | DTSKNTAYLQMNSLRAEDTAVYYCARRHWPGGFDYWGQGTLVTVSSASTKGPSVFPLAPSSKSTSGGGGGGS |
| antibody | DIQMTQSPSSLSASVGDRVTITCRASQDVSTAVAWYQQKPGKAPKLLIYSASFLYSGVPSRFSGSGSGTDFT |
| | LTISSLQPEDFATYYCQQYLYHPATFGQGTKVEIKRTVAAPSVFIGGGGSGGGGSGGGGSGGGGSQ V QLV |
| | QS GGGL V QPGGS LRLS C A AS GFTF S S Y AMS W VRQ APGKGLEW V S AIS GS GGS |
| | T Y Y ADS VKGRFTIS RDN S KNTLYLQMN S LR AEDT A VY YC AKAPGD YTA YFD |
| | YWGQGTLVTV S SGGGGS EL VLTQS P ATLSLS PGER ATLS CRAS QS VS S YLA W Y |
| | QQKPGQ APRLLIY GAS S RATGIPDRFS GS GS GTDFTLTISRLEPEDFA V YYCQQY GS |
| | SPPYTFGQGTKVEIK |

Primary amion acid Sequence: (GSAM = Linker)

[symbol]

[symbol]

indicates data missing or illegible when filed

In some embodiments of the compound of Formula (II) or Formula (III), X₂ is a linker, which can take many forms as shown herein. In some embodiments of the compound of Formula (II) or Formula (III), X₂ is a cleavable linker. In some embodiments of the compound of Formula (II) or Formula (III), X₂ is a non-cleavable linker. In some embodiments of the compound of Formula (II) or Formula (III), X₂ is a mixture of X_{2b} and X_{2c} c, wherein X_{2b} is a linker that is bound to one Cys residue on X₁ and X₂ is a linker that is bound to two Cys residues on X₁, wherein n is a combination of n1 and n2, wherein n1 corresponds to the number of X_{2b} moieties bound to X₁ and n2 corresponds to the number of X_{2c} moieties bound to X₁, and wherein the sum of n1 and n2 is 2, 3, 4, 5, 6, 7, 8, 9, 10, or 11. In some embodiments, each X₂ bound to X₁ is an X_{2b} moiety. In some embodiments, each X₂ bound to X₁ is an X_{2c} moiety. In some embodiments of the compound of Formula (II) or Formula (III), X₂ is a linker listed in Table 5, wherein the left side of X₂, as drawn, is bound to X₁, and the right side of X₂, as drawn, is bound to X₃. In Table 5, R=H, alkyl, aryl, arylalkyl, glycol ether, or an additional glycol linker attaching to the therapeutic compound.

TABLE 5

| Linker | Structure |
|---|---|
| K-1 Cleavable Linker | 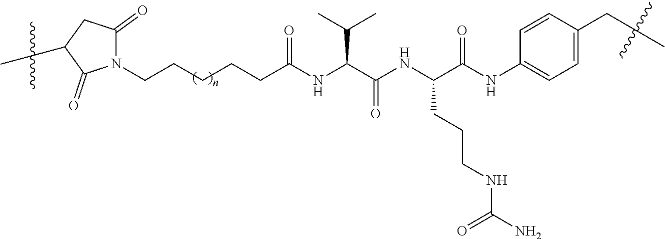 |
| K-2 Cleavable Linker | 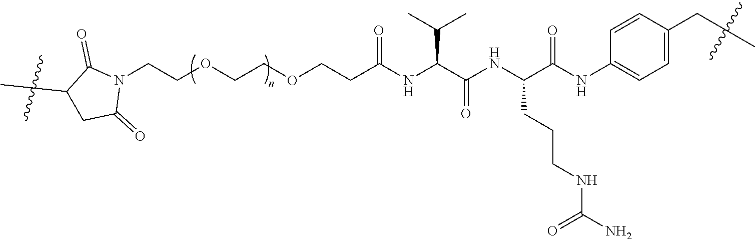 |
| K-3 Cleavable Linker | 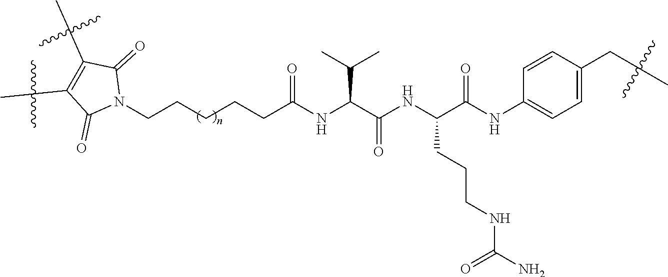 |
| K-4 Cleavable Linker | 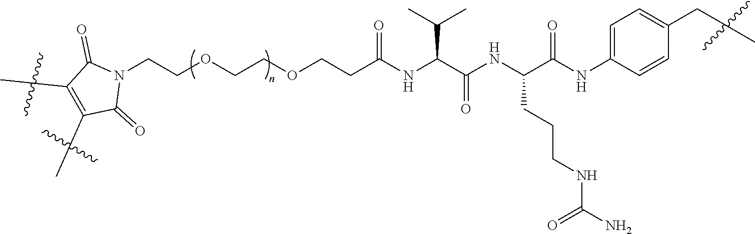 |
| K-5 Cleavable Linker | 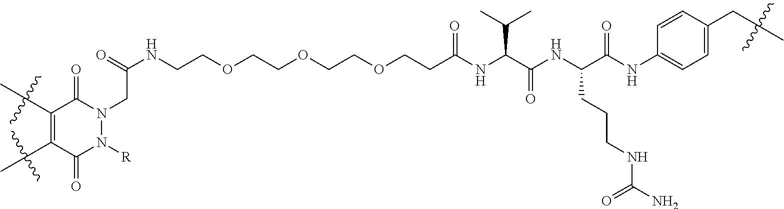 |
| K-6 Cleavable Linker | 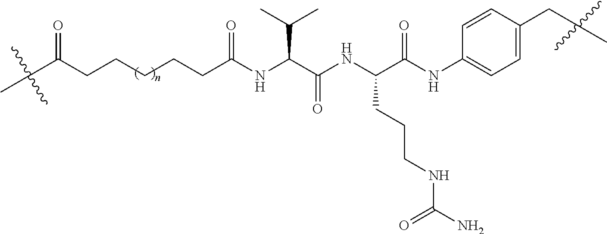 |
| K-7 Cleavable Linker | 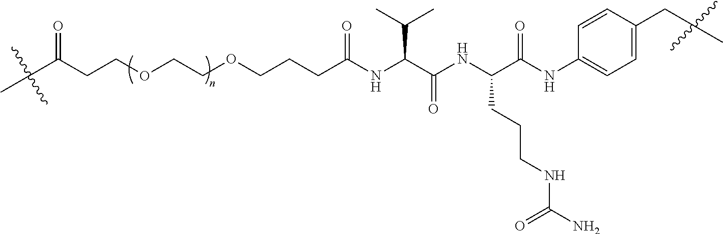 |
| K-8 Cleavable Linker | 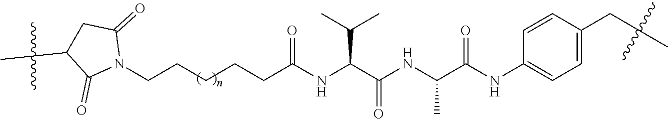 |
| K-9 Cleavable Linker | 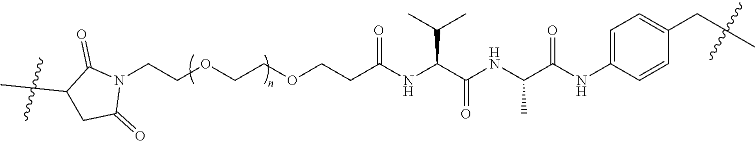 |
| K-10 Cleavable Linker | 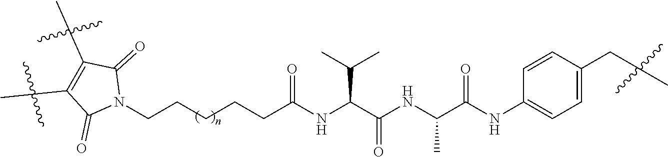 |
| K-11 Cleavable Linker | 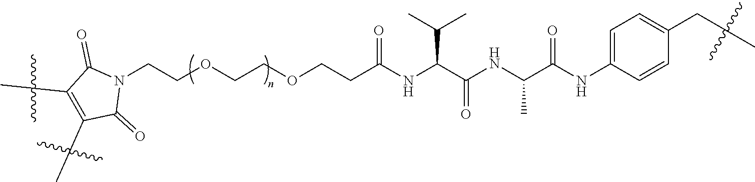 |
| K-12 Cleavable Linker | 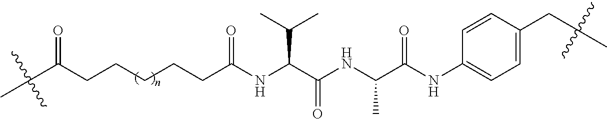 |
| K-13 Cleavable Linker | 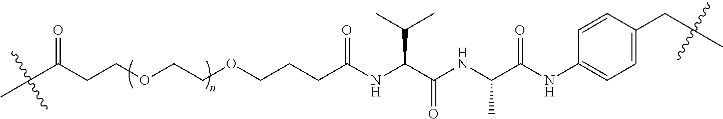 |
| K-14 Cleavable Linker | 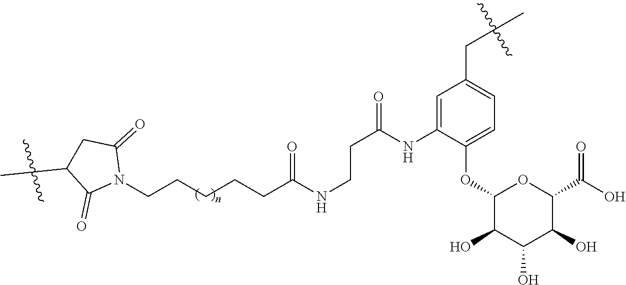 |
| K-15 Cleavable Linker | 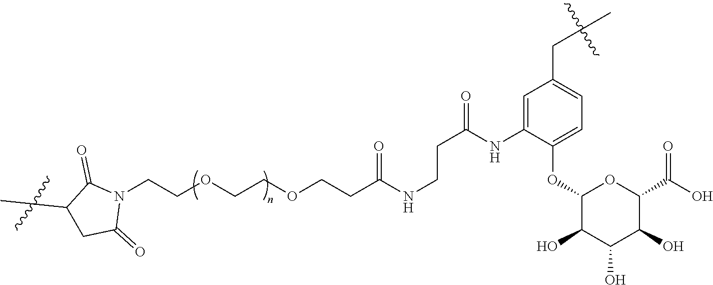 |
| K-16 Cleavable Linker | 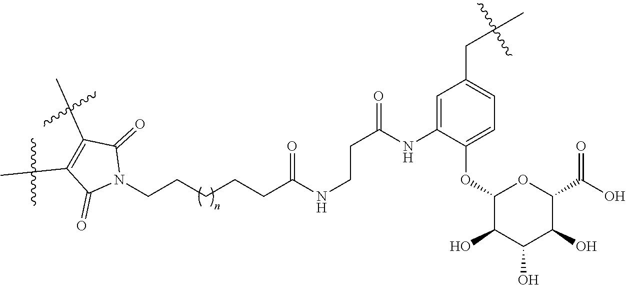 |
| K-16 Cleavable Linker | 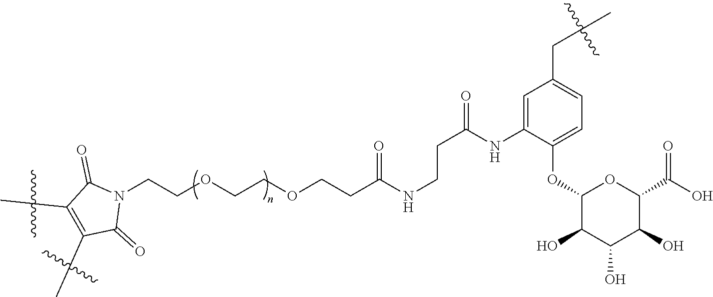 |
| K-17 Cleavable Linker | 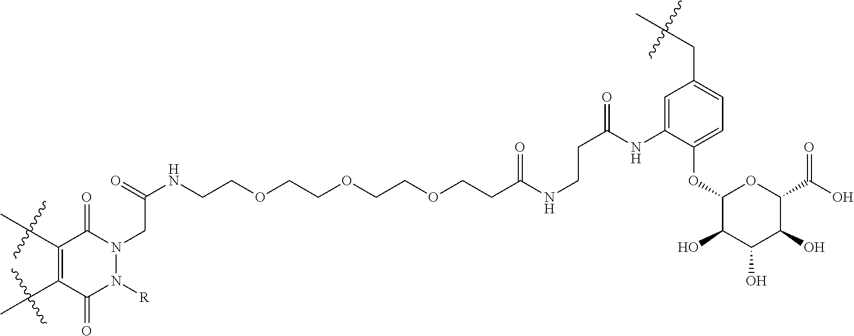 |
| K-18 Cleavable Linker | 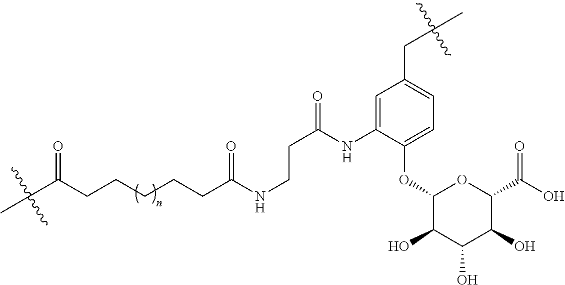 |
| K-19 Cleavable Linker | 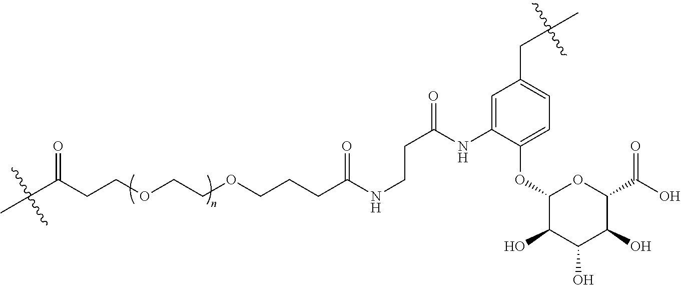 |
| K-20 Non- Cleavable Linker | 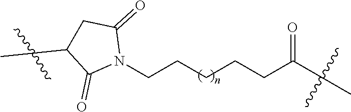 |
| K-21 Non- Cleavable Linker | 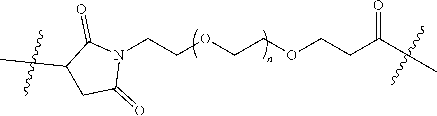 |
| K-22 Non- Cleavable Linker | 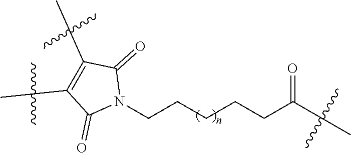 |
| K-23 Non- Cleavable Linker | 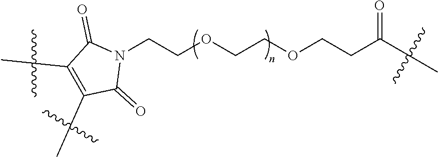 |
| K-24 Non- Cleavable Linker | 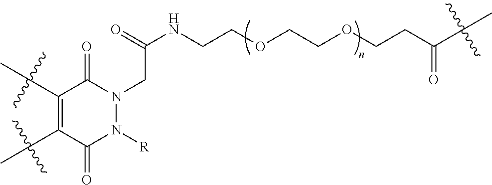 |
| K-25 Non- Cleavable Linker | 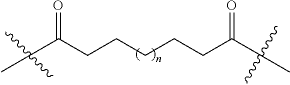 |
| K-26 Non- Cleavable Linker | 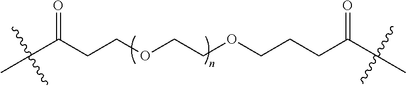 |

In some embodiments of the compound of Formula (II) or Formula (III), X₂ is a non-cleavable linker selected from the group consisting of K-20, K-21, K-22, K-23, K-24, K-25 and K-26.

In some embodiments of the compound of Formula (II) or Formula (III), X₂ is a mixture of X_{2b} and X_{2c}, wherein X_{2b} is a linker that is bound to one Cys residue on X₁ and X₂ is a linker that is bound to two Cys residues on X₁, n is a combination of n1 and n2, wherein n1 corresponds to the number of X_{2b} moieties bound to X₁ and n2 corresponds to the number of X_{2c} moieties bound to X₁, and wherein the sum of n1 and n2 is 2, 3, 4, 5, 6, 7, 8, 9, 10, or 11. In certain such embodiments, X_{2b} is K-1 and X₂ is K-19. In some embodiments of the compound of Formula (II) or Formula (III), wherein X₂ is a mixture of X_{2b} and X_{2c} c, X_{2b} is selected from the group consisting of K-1, K-2, K-3, K-4, K-5, K-8, K-9, K-10, K-11, K-14, K-15, and K-16 and X_{2c} is K-17. In some embodiments, each X₂ bound to X₁ is an X_{2b} moiety. In some embodiments, each X₂ bound to X₁ is an X_{2c} moiety.

In some embodiments of the compound of Formula (II) or Formula (III), X₃ is a therapeutic compound as detailed herein.

In some embodiments, the compound of Formula (II) or Formula (III) is a compound listed in Tables 6, or a pharmaceutically acceptable salt, solvate, hydrate, isomer, or tautomer thereof, wherein X₁ is a biologically active polypeptide or hormone or if the compound is of Formula (III), an Insulin polypeptide or a bio-active homolog polypeptide thereof, and n is 1, 2, 3, 4, 5, 6, 7, 8, 9, 10 or 11. In Table 6, R=H, alkyl, aryl, arylalkyl, glycol ether, or an additional glycol linker attaching to the therapeutic compound; q is 1, 2, 3, or 4; n1 is 1, 2, 3, 4, 5, 6, 7, 8, 9, or 10; and n2 is 1, 2, 3, 4, 5, 6, 7, 8, 9, or 10, wherein the sum of n1 and n2 is 2, 3, 4, 5, 6, 7, 8, 9, 10, or 11.

TABLE 6
Exemplary Compounds of Formula (II) or Formula (III)

| Com- | |
|---|---|
| pound | Structure |
| B-1 | 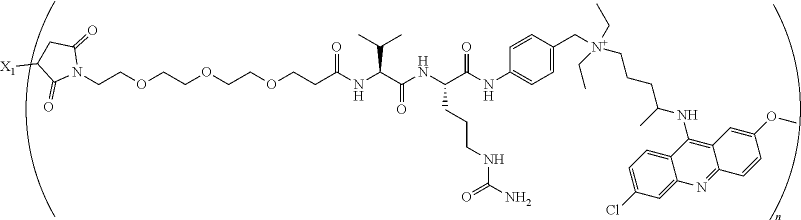 |
| B-2 | 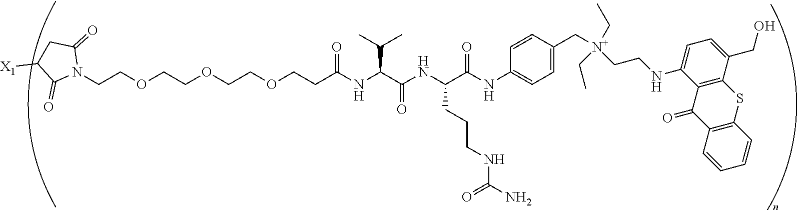 |
| B-3 | 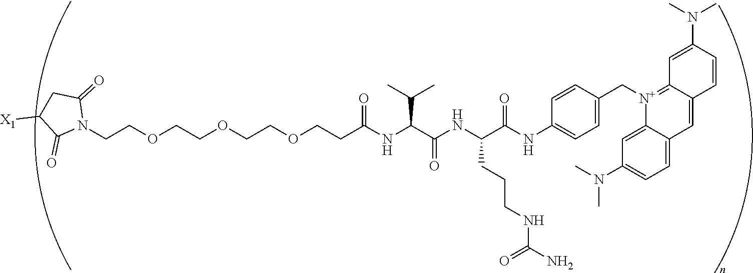 |
| B-4 | 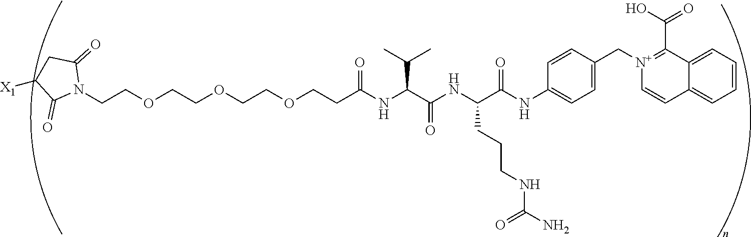 |
| B-5 | 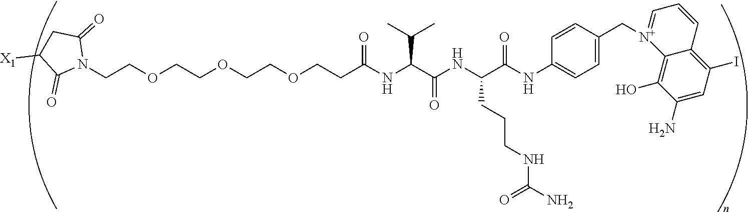 |
| B-6 | 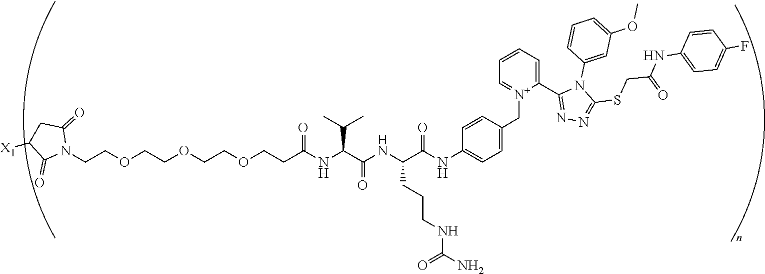 |
| B-7 | 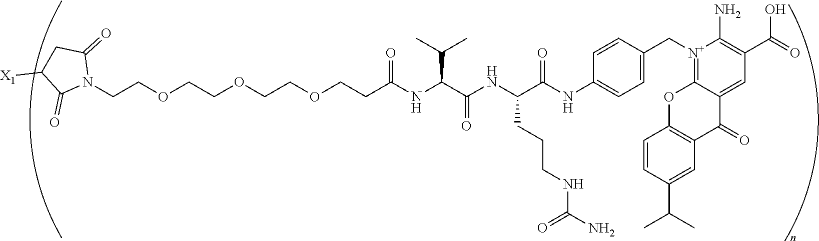 |
| B-8 | 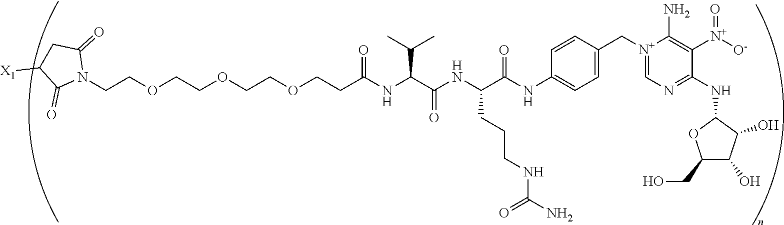 |
| B-9 | 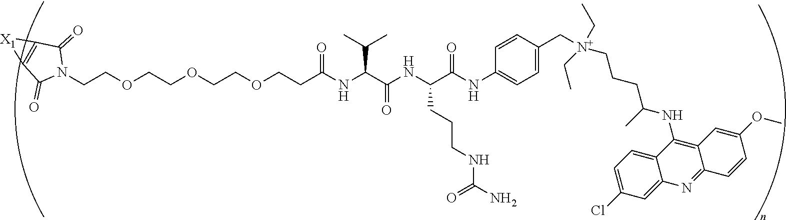 |
| B-10 | 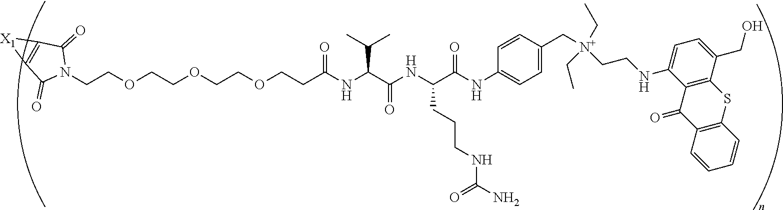 |
| B-11 | 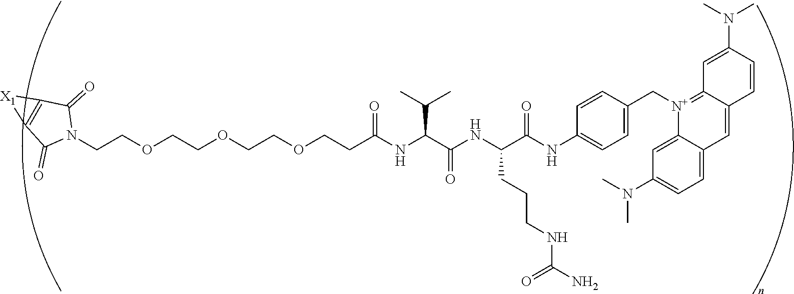 |
| B-12 | 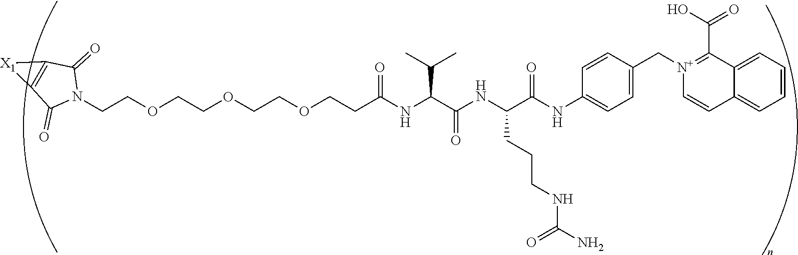 |
| B-13 | 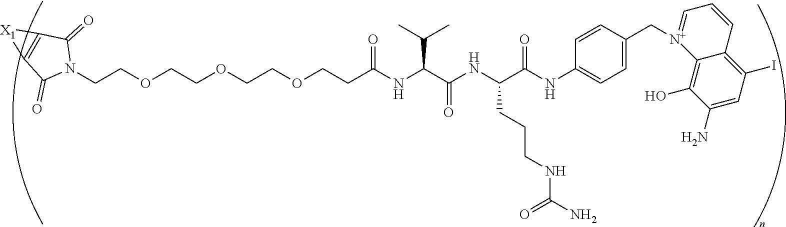 |
| B-14 | 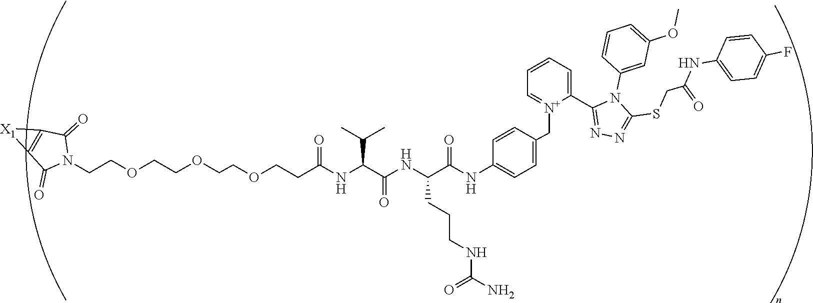 |
| B-15 | 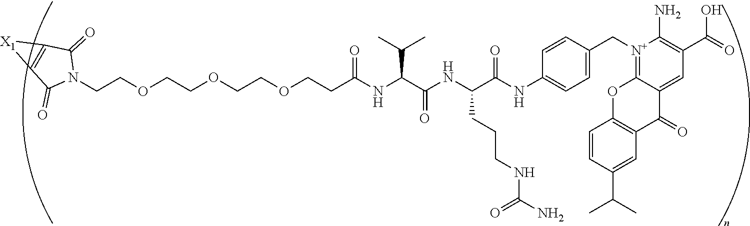 |
| B-16 | 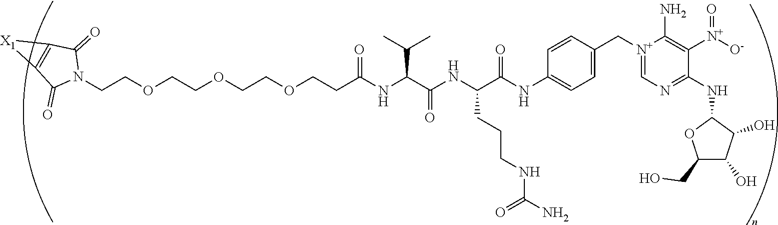 |
| B-17 | 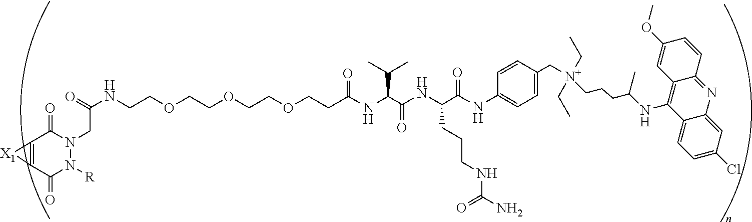 |
| B-18 | 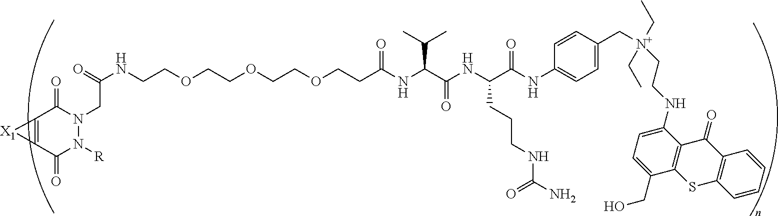 |
| B-19 | 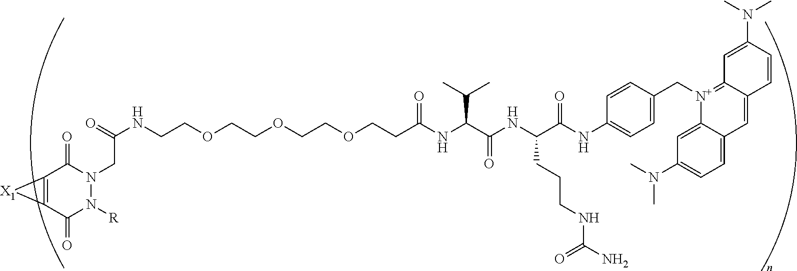 |
| B-20 | 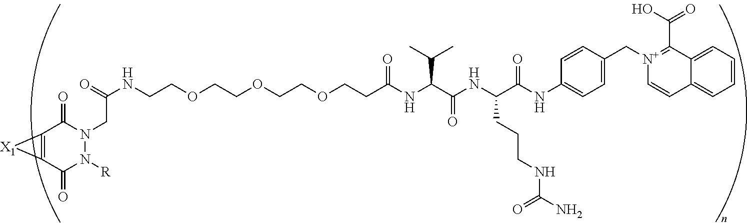 |
| B-21 | 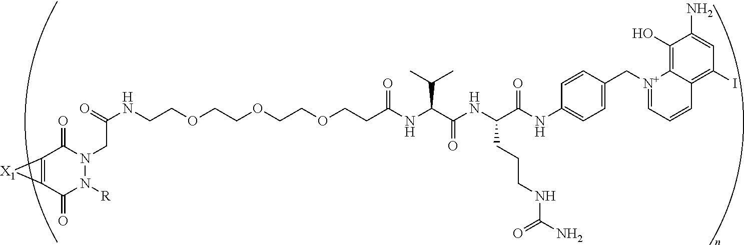 |
| B-22 | 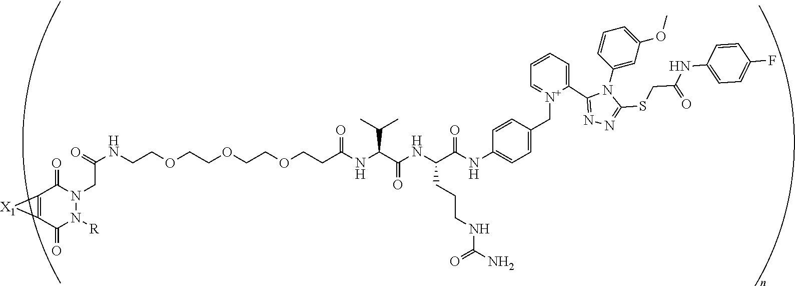 |
| B-23 | 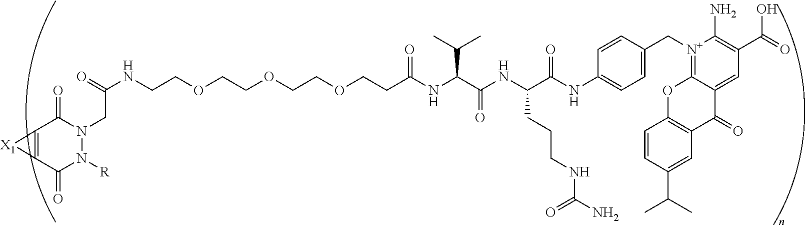 |
| B-24 | 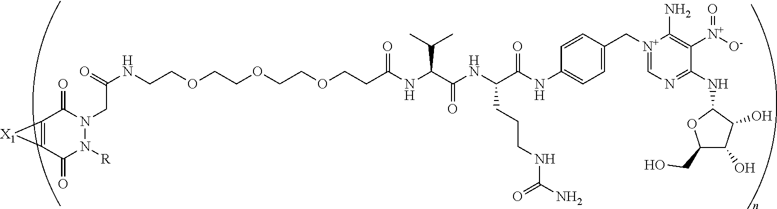 |
| B-25 | 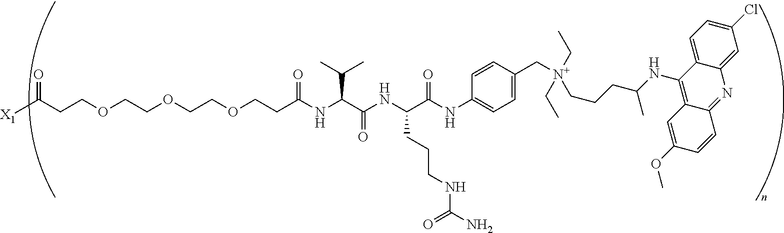 |
| B-26 | 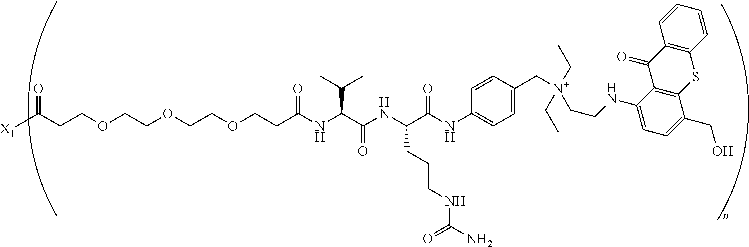 |
| B-27 | 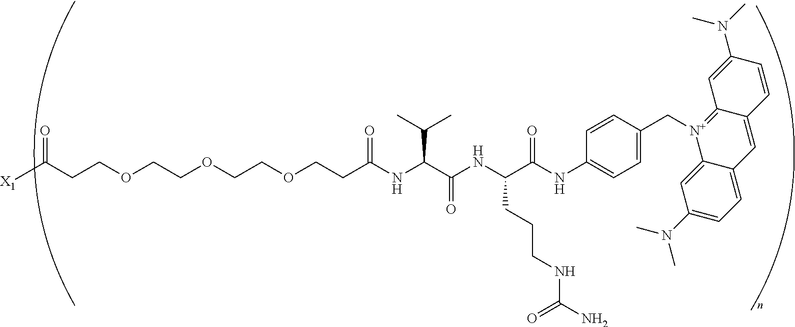 |
| B-28 | 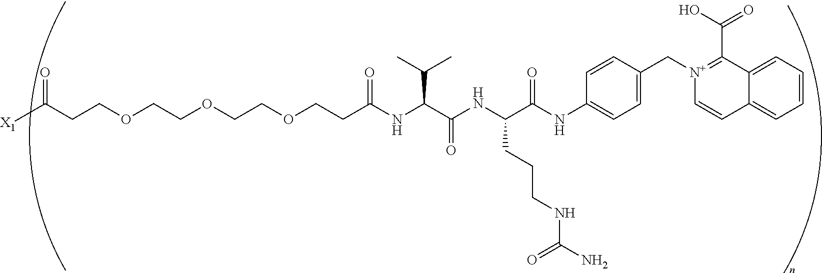 |
| B-29 | 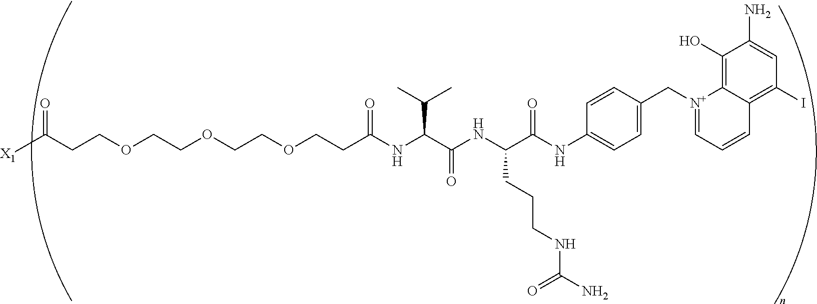 |
| B-30 | 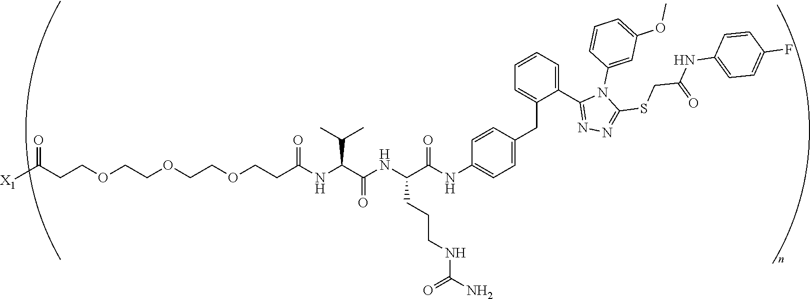 |
| B-31 | 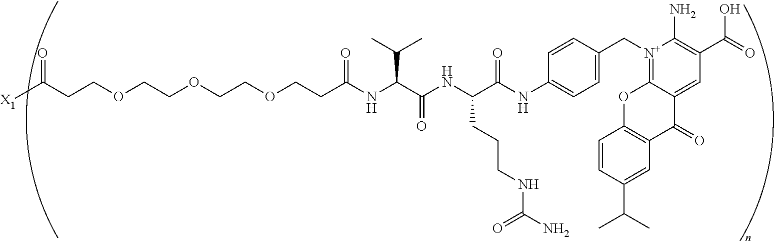 |
| B-32 | 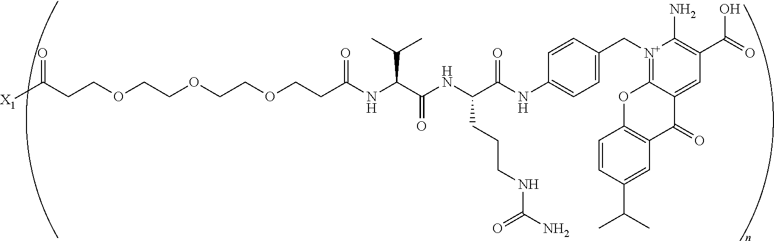 |
| B-33 | 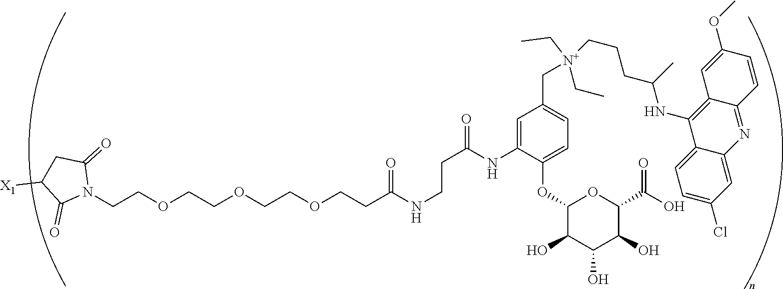 |
| B-34 | 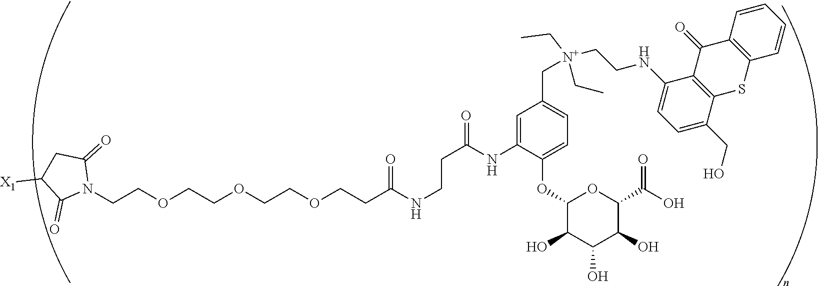 |
| B-35 | 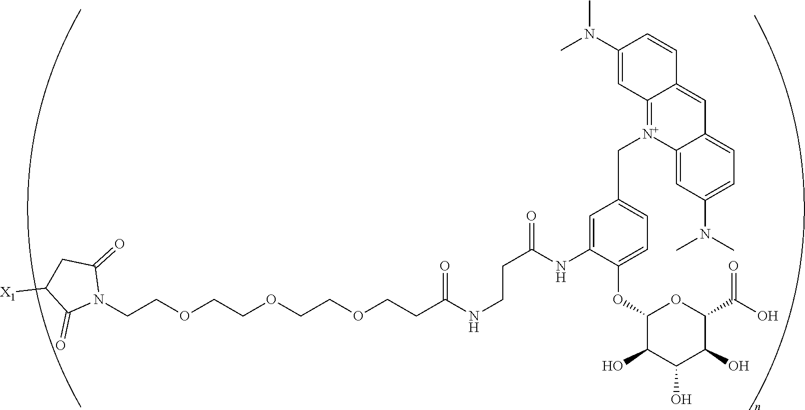 |
| B-36 | 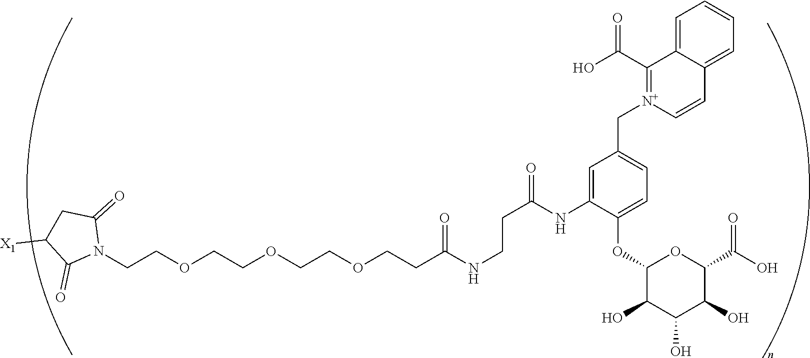 |
| B-37 | 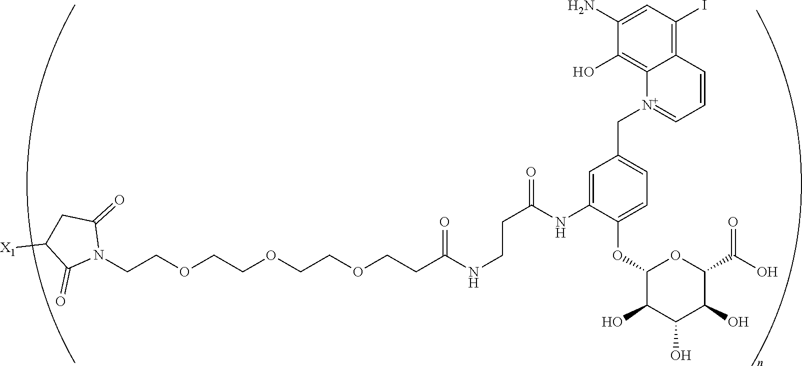 |
| B-38 | 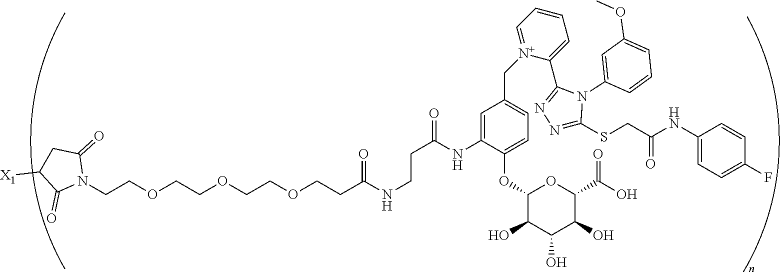 |
| B-39 | 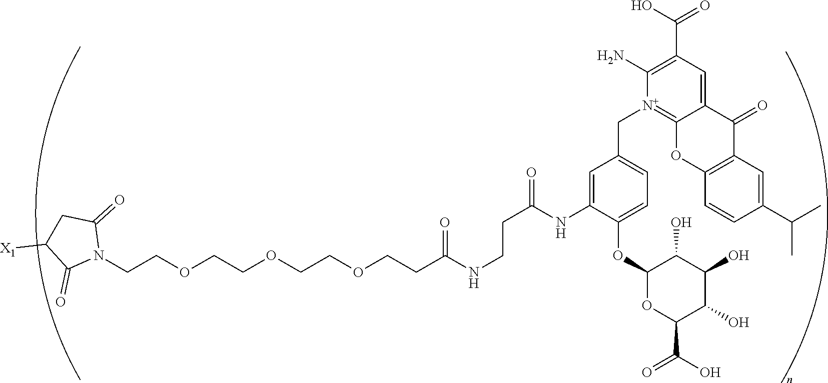 |
| B-40 | 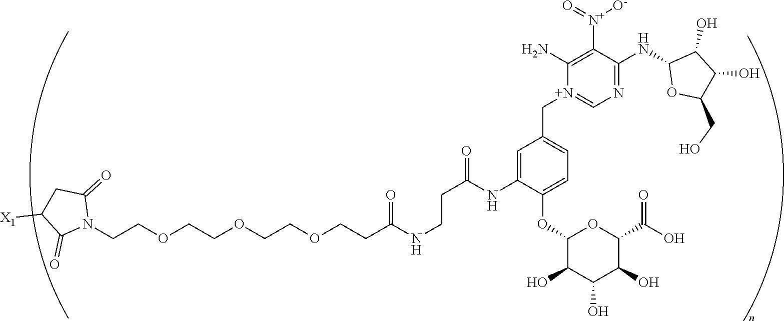 |
| B-41 | 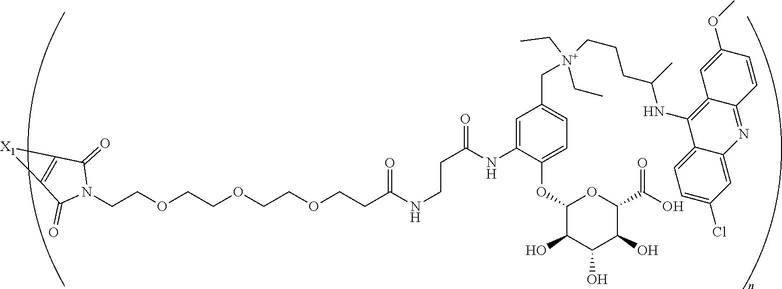 |
| B-42 | 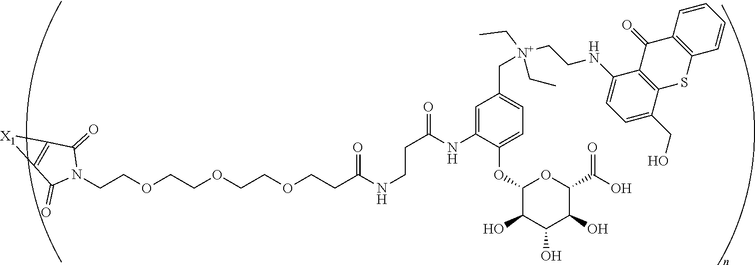 |
| B-43 | 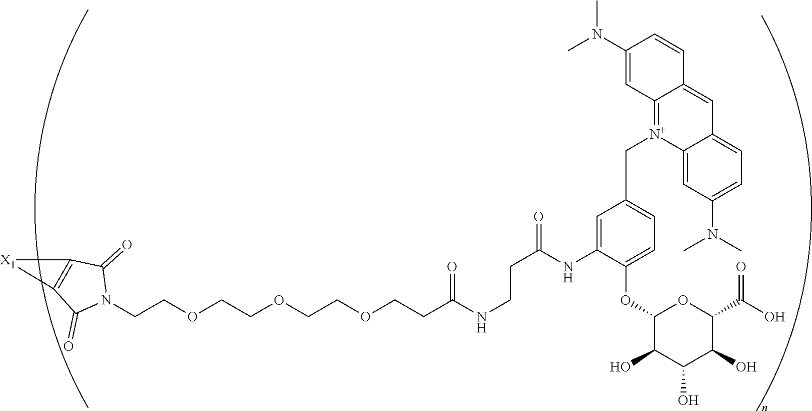 |
| B-44 | 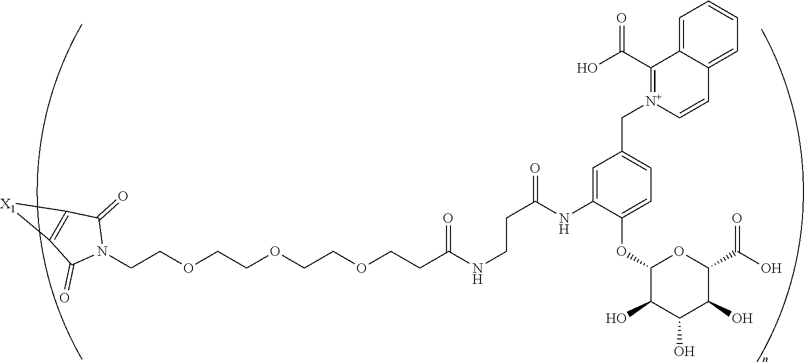 |
| B-45 | 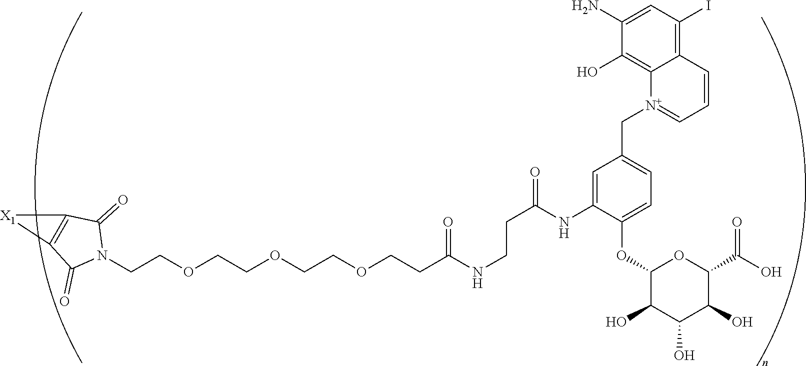 |
| B-46 | 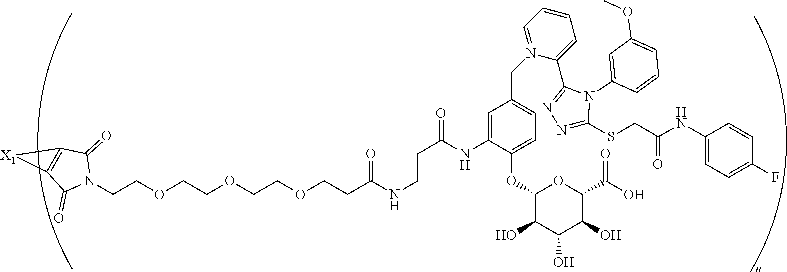 |
| B-47 | 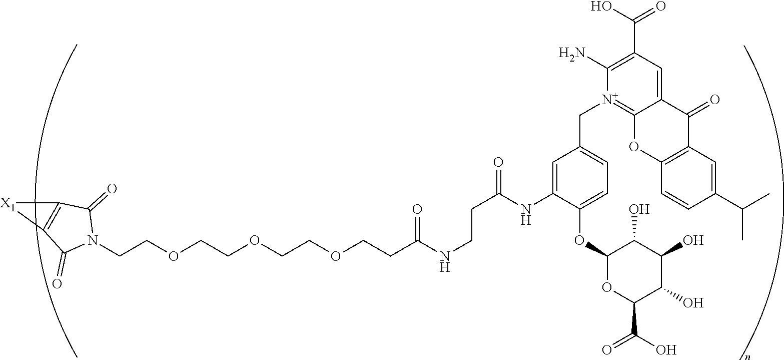 |
| B-48 | 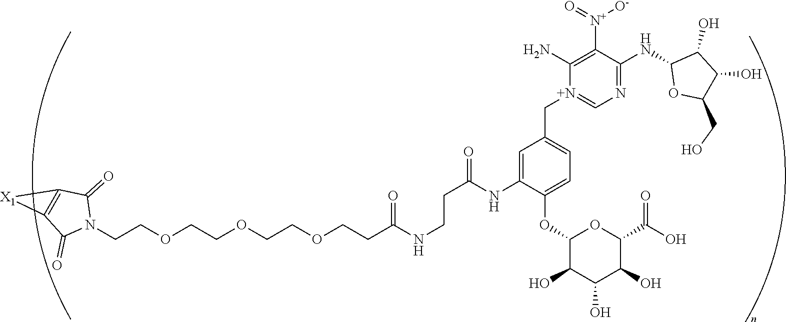 |
| B-49 | 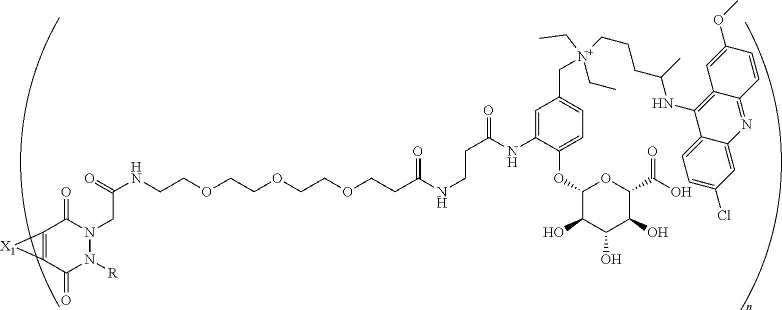 |
| B-50 | 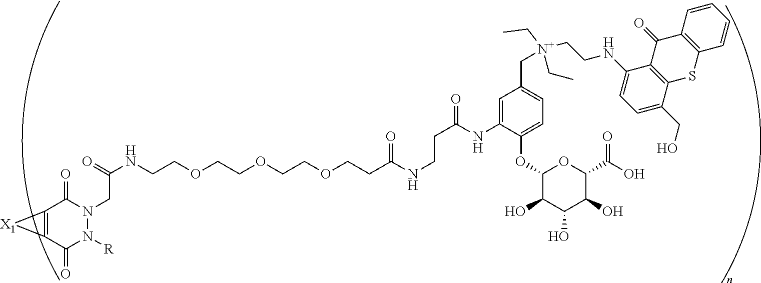 |
| B-51 | 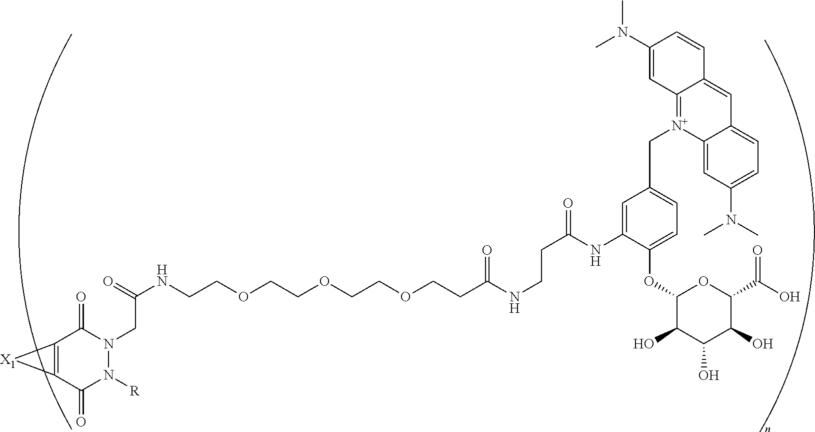 |
| B-52 | 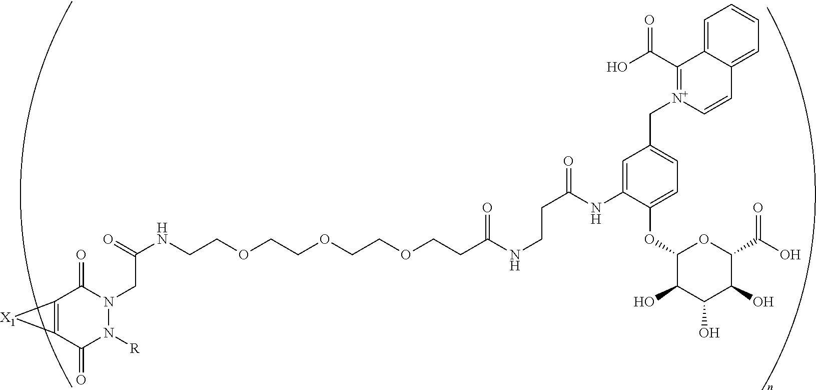 |
| B-53 | 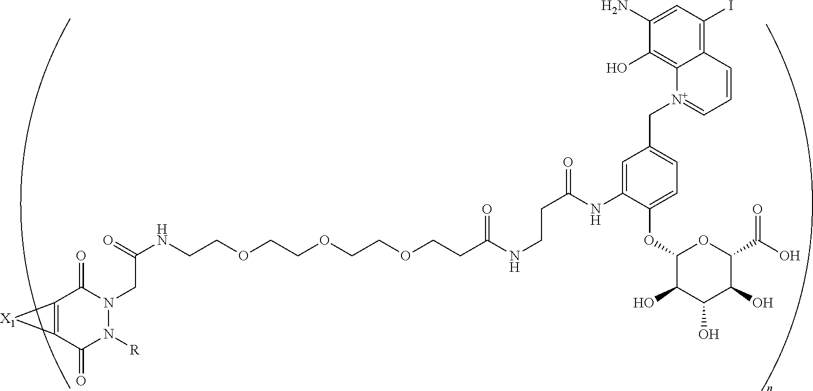 |
| B-54 | 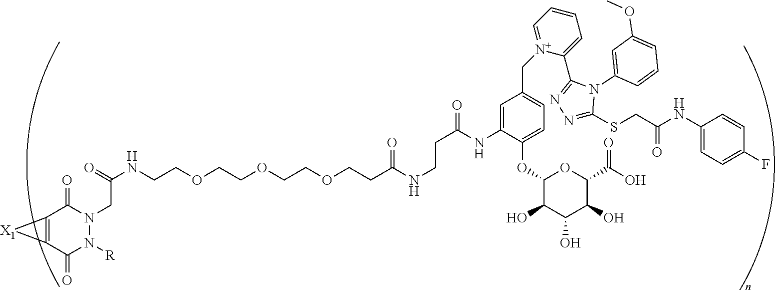 |
| B-55 | 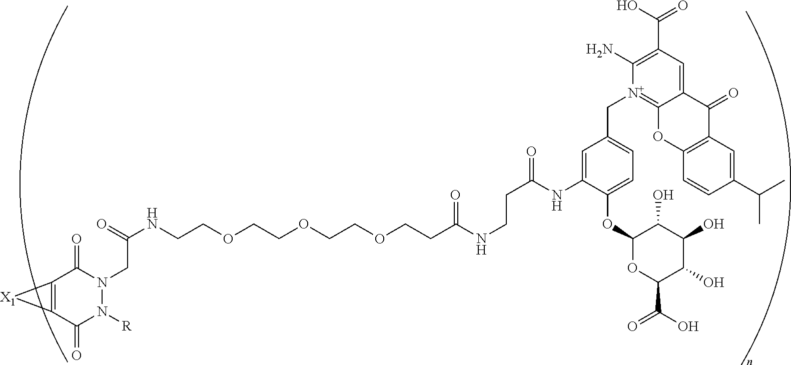 |
| B-56 | 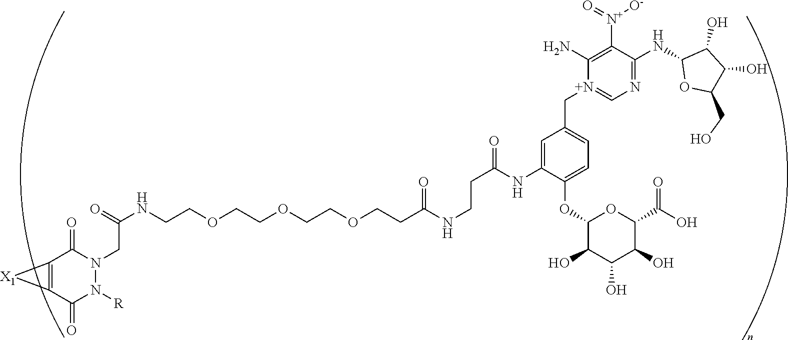 |
| B-57 | 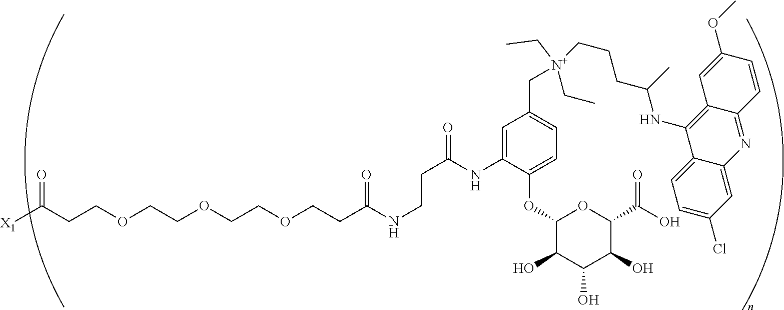 |
| B-58 | 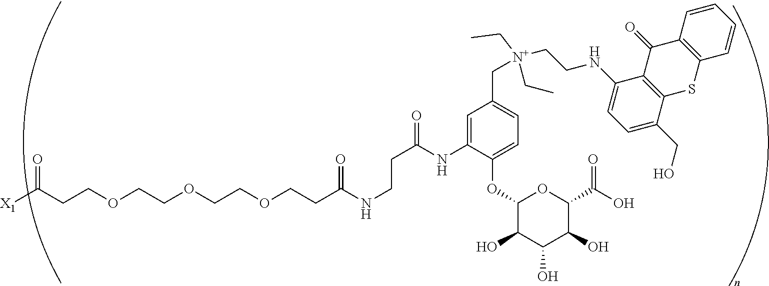 |
| B-59 | 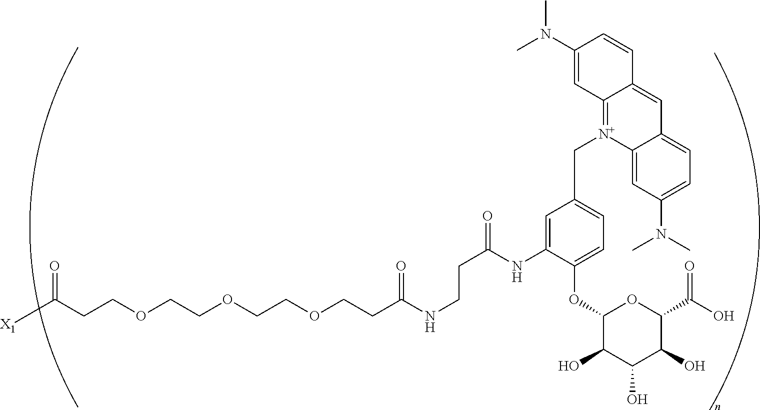 |
| B-60 | 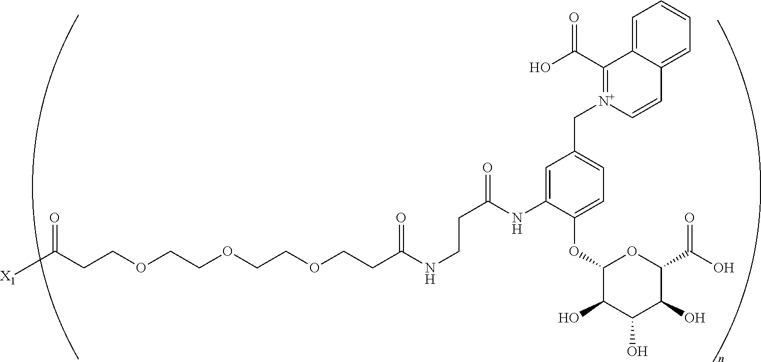 |
| B-61 | 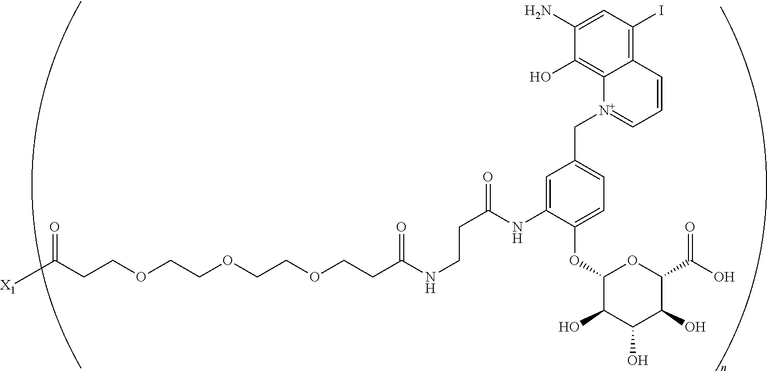 |
| B-62 | 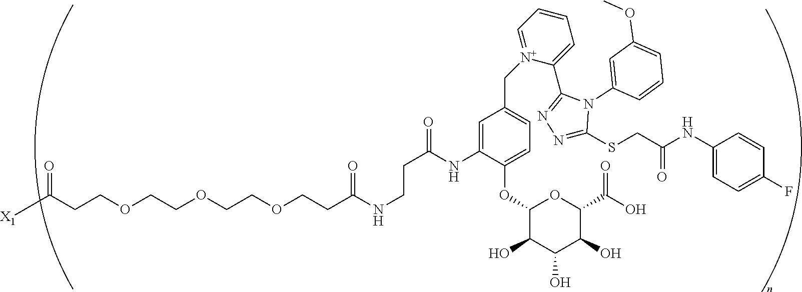 |
| B-63 | 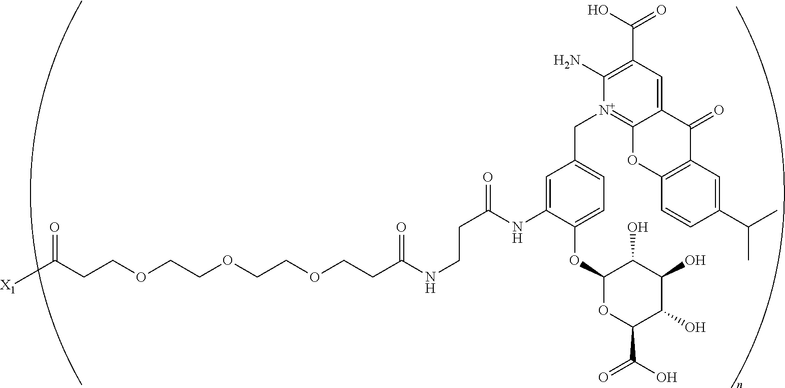 |
| B-64 | 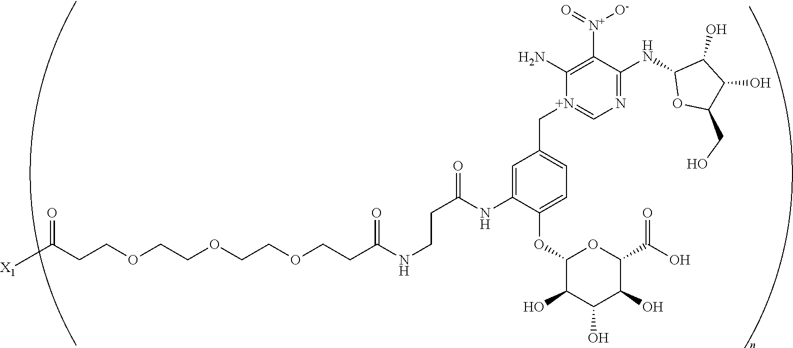 |

### Biological Assays

### Methodology:

We performed the B16 melanoma tumor model experiment according to the method described by Overwijk and Restifo (1) with some modifications. Briefly, 8-week-old female C57BL/6 mice were immunized subcutaneously at the base of the tail with 100 micrograms of ovalbumin mixed with 10 micrograms of LPS (lipopolysaccharide). 8-10 days after immunizations mice (n=5 per group) were injected subcutaneously in the abdomen with 5×105 B16 cells from previously developed B16 cell lines transfected with gene constructs coding for 1. PDL-1 (B16-PDL1) (Table I and III); 2. PDL-1+OVA (B16 OVA-PDL1) (Table II). Starting at the day of the tumor injection (day 0) and then on days 3 and 5, some animals were injected intravenously at the tail vein with 200 micrograms of an anti-T cell cocktail (anti-CD4+anti-CD8) (Table IV). On days 6, 9 and 12 treatment group animals were injected with intravenously with 100 micrograms of either regular anti-PDL1, anti-HVEM and control IgG (Table I and II), or anti-PDL1-ADC (Deruxtecan) (Anti-PDL1-E) anti-HVEM-ADC (Deruxtecan) (Anti-HVEM-E), or a control antibody-ADC (Deruxtecan) (IgG) (Table III and IV). Tumor growth was evaluated every 3 days with the help of a caliper. 30 days after tumor inoculation all animals were euthanized.

In Table V, animals were subjected to the same experimental conditions as in Table III but with the following modifications: antibodies to PDL-1 were the same used in table IV or were modified so that the C-terminal sequence is genetically fused to the full length alpha-melanocortin stimulating hormone peptide (Anti-PDL1-MSH-E). Pre-inoculum B16-PDL1+ tumor cells for each duplicate group (sample 1 and sample 2 (S1 and S2) were RNA sequenced (*) and again from tumor biopsies at day 18 post-inoculation. *Paired-end 50 bps raw reads were obtained from Illumina Hiseq-2500 sequencer. SNV called against mouse reference mRNA (RefSeq RNA).

The results are shown in the data tables below:

TABLE I
B16-PDL1 + tumor

| Tumor | | | |
|---|---|---|---|
| volume | | Anti- | Anti- |
| at day: | IgG | PDL1 | HVEM |
| 0 | 0 | 0 | 0 |
| 3 | 2, 9 | 2, 6 | 3, 1 |
| 6 | 3, 9 | 4, 1 | 3, 5 |
| 9 | 6, 1 | 6, 8 | 7, 2 |
| 12 | 8, 8 | 9, 3 | 8, 6 |
| 15 | 12, 7 | 11, 9 | 11, 4 |
| 18 | 15, 2 | 14, 8 | 14 |
| 21 | 17, 9 | 18, 4 | 17, 8 |
| 24 | 22, 4 | 21, 7 | 21 |
| 27 | 27, 1 | 25, 6 | 23, 7 |
| 30 | 30, 2 | 29, 1 | 28, 9 |

TABLE II
B16-PDL1 + OVA + Tumor

| Tumor | | | |
|---|---|---|---|
| volume | | Anti- | Anti- |
| at day: | IgG | PDL1 | HVEM |
| 0 | 0 | 0 | 0 |
| 3 | 2, 5 | 2, 8 | 2, 2 |
| 6 | 3, 4 | 3, 6 | 3 |
| 9 | 5, 5 | 6, 2 | 5, 9 |
| 12 | 7, 2 | 8, 1 | 8 |
| 15 | 9, 6 | 8 | 9 |
| 18 | 12, 9 | 7, 3 | 13, 6 |
| 21 | 18, 1 | 7, 9 | 16, 9 |
| 24 | 20, 7 | 6, 1 | 19, 7 |
| 27 | 24, 9 | 5, 4 | 22, 6 |
| 30 | 27, 8 | 6 | 25, 8 |

TABLE III
B16-PDL1 + E-ADC treatment

| Tumor | | | |
|---|---|---|---|
| volume | | Anti- | Anti- |
| at day: | IgG | PDL1-E | HVEM-E |
| 0 | 0 | 0 | 0 |
| 3 | 3, 1 | 2, 8 | 3, 3 |
| 6 | 4, 2 | 4, 9 | 4 |
| 9 | 6, 6 | 7, 3 | 7, 2 |
| 12 | 10, 2 | 11, 4 | 10, 8 |
| 15 | 12, 7 | 13, 6 | 12, 9 |
| 18 | 15, 9 | 17, 5 | 16, 8 |
| 21 | 19, 2 | 20, 4 | 22, 7 |
| 24 | 24 | 18 | 25, 3 |
| 27 | 28, 3 | 13, 7 | 29, 4 |
| 30 | 32 | 11, 3 | 33, 1 |

TABLE IV
B16-PDL1 + E-ADC treatment +
anti-T cell depleting antibody cocktail

| Tumor | | | |
|---|---|---|---|
| volume | | Anti- | Anti- |
| at day: | IgG | PDL1-E | HVEM-E |
| 0 | 0 | 0 | 0 |
| 3 | 3, 3 | 2, 8 | 2, 4 |
| 6 | 5, 1 | 4, 6 | 4 |
| 9 | 7, 2 | 7, 6 | 6, 8 |
| 12 | 14, 6 | 13, 1 | 11, 9 |
| 15 | 19, 3 | 17, 4 | 16, 8 |
| 18 | 24, 1 | 25 | 20, 4 |
| 21 | 29, 3 | 28, 8 | 27 |
| 24 | 33, 3 | 31 | 29, 7 |
| 27 | 38, 5 | 36, 2 | 32, 6 |
| 30 | 41 | 39, 9 | 36, 2 |

TABLE V
Single nucleotide variation number

| | Pre- | |
|---|---|---|
| | inoculum | Day 18 |
| Treatment | tumor | tumor |
| | 0 | 18 |
| IgG-S1 | 17 | 19 |
| IgG-S2 | 26 | 24 |
| Anti-PDL1-E-S1 | 27 | 63 |
| Anti-PDL1-E-S2 | 19 | 52 |
| Anti-PDL1- | 22 | 118 |
| MSH-E-S1 | | |
| Anti-PDL1- | 15 | 131 |
| MSH-E-S2 | | |

In some embodiments, the disclosure is directed to compounds as described herein and pharmaceutically acceptable salts, solvates, hydrates, isomers, or tautomers thereof. The use of the terms “salt,” “hydrate,” “solvate,” and the like, is intended to equally apply to the salt, hydrate, or solvate of isomers, tautomers, or racemates of the disclosed compounds.

It should be understood that all isomeric forms are included within the present disclosure, including mixtures thereof. The term “isomer” may refer to compounds that have the same composition and molecular weight but differ in physical and/or chemical properties. The structural difference may be in constitution (geometric or positional isomers) or in the ability to rotate the plane of polarized light (stereoisomers). With regard to stereoisomers, the compounds of the disclosure may have one or more asymmetric carbon atom and may occur as racemates, racemic mixtures and as individual enantiomers or diastereomers. Individual isomers of the compounds of the disclosure may, for example, be substantially free of other isomers, or may be admixed, for example, as racemates or with all other, or other selected, isomers. If the compound contains a double bond, the substituent may be in the E or Z configuration or cis or trans configuration or mixtures of any of the foregoing. Disclosed assay results may reflect the data collected for the racemic form, the enantiomerically pure form, or any other form in terms of stereochemistry or constitution (e.g., geometric or positional isomers).

The compounds of the disclosure may contain asymmetric or chiral centers, and, therefore, exist in different stereoisomeric forms. The term “stereoisomers” may refer to the set of compounds which have the same number and type of atoms and share the same bond connectivity between those atoms, but differ in the three-dimensional structure. The term “stereoisomer” may refer to any member of this set of compounds. For instance, a stereoisomer may be an enantiomer or a diastereomer. It is intended that all stereoisomeric forms of the compounds of the disclosure as well as mixtures thereof, including racemic mixtures, form part of the present disclosure.

The term “enantiomers” may refer to a pair of stereoisomers which are non-superimposable mirror images of one another. The term “enantiomer” may refer to a single member of this pair of stereoisomers. The term “racemic” may refer to a 1:1 mixture of a pair of enantiomers. Each compound herein disclosed may include all the enantiomers (which may exist even in the absence of asymmetric carbons) that conform to the general structure of the compound, unless the stereochemistry is specifically indicated. The compounds may be in a racemic or enantiomerically pure form, or any other form in terms of stereochemistry. The chiral centers of the present disclosure may have the S or R configuration as defined by the IUPAC 1974 Recommendations. In some examples presented, the synthetic route may produce a single enantiomer or a mixture of enantiomers. In some embodiments of the disclosure, the compounds of the disclosure are enantiomers. In some embodiments, the compounds of the disclosure are the (S)-enantiomer. In other embodiments, the compounds of the disclosure are the (R)-enantiomer. In yet other embodiments, the compounds of the disclosure may be (+) or (−) enantiomers.

The term “diastereomers” may refer to the set of stereoisomers which cannot be made superimposable by rotation around single bonds. For example, cis- and trans-double bonds, endo- and exo-substitution on bicyclic ring systems, and compounds containing multiple stereogenic centers with different relative configurations may be considered to be diastereomers. The term “diastereomer” may refer to any member of this set of compounds. In some examples presented, the synthetic route may produce a single diastereomer or a mixture of diastereomers. The disclosure may include diastereomers of the compounds described herein.

In some embodiments, pharmaceutical compositions of the disclosure may be enriched to provide predominantly one enantiomer of a compound described herein. An enantiomerically enriched mixture may comprise, for example, at least 60 mol percent of one enantiomer, or more preferably at least 75, at least 80, at least 85, at least 90, at least 95, at least 96, at least 97, at least 98, at least 99, at least 99.5 or even 100 mol percent. In some embodiments, the compound described herein enriched in one enantiomer may be substantially free of the other enantiomer, wherein substantially free may mean that the substance in question makes up less than 10%, or less than 5%, or less than 4%, or less than 3%, or less than 2%, or less than 1% as compared to the amount of the other enantiomer, e.g., in the pharmaceutical composition or compound mixture. For example, if a pharmaceutical composition or compound mixture contains 98 grams of a first enantiomer and 2 grams of a second enantiomer, it would be said to contain 98 mol percent of the first enantiomer and only 2 mol percent of the second enantiomer.

In some embodiments, the pharmaceutical compositions of the disclosure may be enriched to provide predominantly one diastereomer of a compound disclosed herein. A diastereomerically enriched mixture may comprise, for example, at least 60 mol percent of one diastereomer, or more preferably at least 75, at least 80, at least 85, at least 90, at least 95, at least 96, at least 97, at least 98, at least 99, at least 99.5, or even 100 mol percent. In some embodiments, the compound described herein enriched in one diastereomer may be substantially free of other diastereomers, wherein substantially free may mean that the substance in question makes up less than 10%, or less than 5%, or less than 4%, or less than 3%, or less than 2%, or less than 1% as compared to the amount of other disastereomers, e.g., in the pharmaceutical composition or compound mixture.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column. Also, some of the compounds of the disclosure may be atropisomers or rotameric forms and are considered as part of this disclosure.

Compounds of the disclosure may exist in their tautomeric form (for example, as an amide or imino ether). All such tautomeric forms are contemplated herein as part of the present disclosure.

In some embodiments, the present disclosure provides compounds of Formula (I), (II), or (III), or pharmaceutically acceptable salts, solvates, hydrates, or tautomers thereof, or pharmaceutical compositions comprising the compounds, or pharmaceutically acceptable salts, solvates, hydrates, or tautomers thereof, wherein the isomeric form and/or stereochemistry is not determined. All isomers, including stereoisomers, of Formula (I), (II), or (III), or pharmaceutically acceptable salts, solvates, hydrates, or tautomers thereof, are hereby included in the disclosure.

The disclosure may include pharmaceutically acceptable salts of the compounds disclosed herein. A “pharmaceutically acceptable salt” may be acceptable for use in humans or domestic animals and may refer to those salts that retain the biological effectiveness and properties of the free forms, which are not biologically or otherwise undesirable. Representative “pharmaceutically acceptable salts” may include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, 1,1-methene-bis-2-hydroxy-3-naphthoate, einbonate, pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

Pharmaceutically acceptable salts may also include both acid and base addition salts. “Pharmaceutically acceptable acid addition salt” may refer to those salts which retain the biological effectiveness and properties of the free bases, which are not biologically or otherwise undesirable, and which may be formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” may refer to those salts that retain the biological effectiveness and properties of the free acids, which are not biologically or otherwise undesirable. These salts may be prepared from addition of an inorganic base or an organic base to the free acid. Salts derived from inorganic bases may include, but are not limited to, the sodium, potassium, lithium, ammonium, calcium, magnesium, iron, zinc, copper, manganese, aluminum salts and the like. For example, inorganic salts may include, but are not limited to, ammonium, sodium, potassium, calcium, and magnesium salts. Salts derived from organic bases may include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like.

Compounds of the disclosure may exist as solvates. The term “solvate” may refer to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates may include compositions containing stoichiometric amounts of water, as well as compositions containing variable amounts of water.

The compounds described herein further include all pharmaceutically acceptable isotopically labeled compounds. An “isotopically” or “radio-labeled” compound may be a compound where one or more atoms are replaced or substituted by an atom having an atomic mass or mass number different from the atomic mass or mass number typically found in nature (i.e., naturally occurring). For example, in some embodiments, in the compounds described herein hydrogen atoms are replaced or substituted by one or more deuterium or tritium. Certain isotopically labeled compounds of this disclosure, for example, those incorporating a radioactive isotope, may be useful in drug and/or substrate tissue distribution studies. The radioactive isotopes tritium, i.e., ³H, and carbon 14, i.e., ¹⁴C, may be particularly useful for this purpose in view of their ease of incorporation and ready means of detection. Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances. Suitable isotopes that may be incorporated in compounds described herein include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I. Substitution with positron emitting isotopes, such as ¹¹C, ¹⁸F, ¹⁵O, and ¹³N, can be useful in Positron Emission Topography (PET) studies.

### Methods of Synthesizing the Compounds

The compounds of the present disclosure (e.g., a compound of Formula (I), (II), or (III)), or pharmaceutically acceptable salts, hydrates, solvates, isomers, or tautomers thereof, may be made by a variety of methods, including standard chemistry. Suitable synthetic routes are depicted in the schemes given herein.

The compounds disclosed herein (e.g., a compound of Formula (I), (II), or (III)), or pharmaceutically acceptable salts, hydrates, solvates, isomers, or tautomers thereof, may be prepared by methods known in the art of organic synthesis as set forth in part by the following synthetic schemes. In the schemes described herein, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis,” Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of disclosed herein (e.g., a compound of Formula (I), (II), or (III)), or pharmaceutically acceptable salts, hydrates, solvates, isomers, or tautomers thereof.

Those skilled in the art will recognize if a stereocenter exists in the compounds disclosed herein (e.g., a compound of Formula (I), (II), or (III)), or pharmaceutically acceptable salts, hydrates, solvates, isomers, or tautomers thereof. Compounds disclosed herein can exist as enantiomeric or diastereomeric stereoisomers. Accordingly, the present disclosure includes all possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. For example, enantiomerically pure compounds of the disclosure can be prepared using enantiomerically pure chiral building blocks. Alternatively, racemic mixtures of the final compounds or a racemic mixture of an advanced intermediate can be subjected to chiral purification as described herein to deliver the desired enantiomerically pure intermediates or final compounds. In the instances where an advanced intermediate is purified into its individual enantiomers, each individual enantiomer can be carried on separately to deliver the final enantiomerically pure compounds of the disclosure. Resolution of the final product, an intermediate, or a starting material may be affected by any suitable method known in the art. See, for example, “Stereochemistry of Organic Compounds,” by E. L. Eliel, S. H. Wilen, and L. N. Mander (Wiley-lnterscience, 1994). This reference is incorporated by reference in its entirety

The compounds described herein (e.g., a compound of Formula (I), (II), or (III)), or pharmaceutically acceptable salts, hydrates, solvates, isomers, or tautomers thereof, may be made from commercially available starting materials or synthesized using known organic, inorganic, and/or enzymatic processes.

### Dipeptide Linkers

In some cases, the dipeptide based linkers described herein have been utilized as cleavable groups for the delivery of therapeutic compound payloads in other constructs to cells or tissue of interest. Several combinations of dipeptides have been utilized, with the valine-citrulline (Val-Cit) and valine-alanine (Val-Ala) as the most successful combinations. [13, 14, 15, 16, 17] The synthetic pathway leading to the synthesis of Val-Cit and Val-Ala dipeptides are very similar and shown in the Scheme 1 herein. The amino group of Valine is initially protected with a Fluorenylmethyloxycarbonyl (Fmoc) group, followed by the activation of the carboxylic acid group with N-Hydroxysuccinimide (NHS) to form compound 3. Compound 3 can then be reacted with either citrulline or alanine in an aqueous solution of NaHCO₃ with DMF and THF present to ensure proper solubility. The resulting compound 4 can be coupled to para-aminobenzylalcohol group (PABOH) using N-ethoxycarbonyl-2-ethoxy-1,2-dihydroquinoline (“EEDQ”), which leads to the formation of compound 5.[18] Removal of the Fmoc group may be carried out with diethyl amine in DMF at room temperature to give amino-alcohol 6.

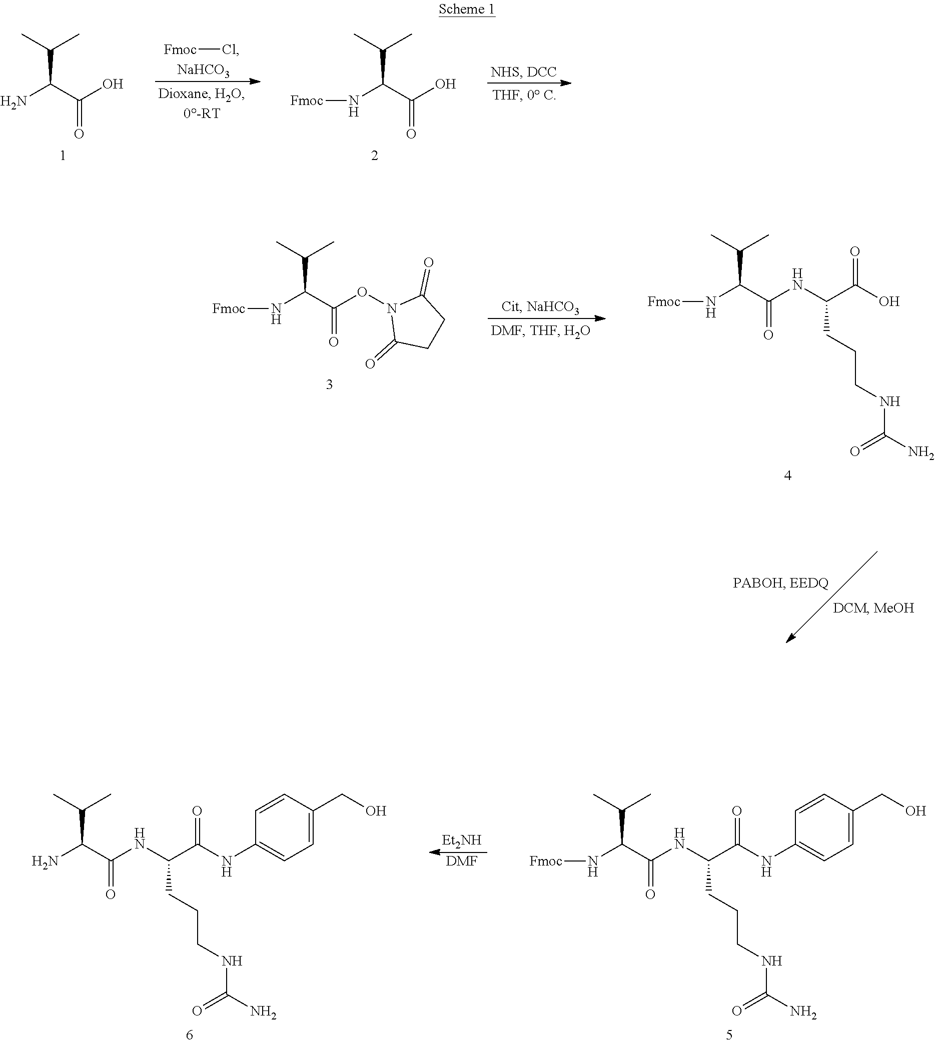

As shown in Scheme 2, glycols of various lengths may be reacted with a catalytic amount of sodium metal under anhydrous condition to promote the addition to tert-butyl acrylate for the Michael Addition product 9.[19] Next, maleimide can be reacted with glycol portion of 9 under Mitsunobu conditions to produce compound 10 in good yields.[20] Deprotection of the tert-butyl ester with trifluoroacetic acid may be followed by the esterification under Steglich conditions with DIC and pentafluorophenol to give compound 12.[21] An alternative approach to the formation of the pentafluorophenol ester is the reaction of the carboxylic acid with perfluorophenyl 2,2,2-trifluoroacetate (PPTA) in the presence of pyridine in DMF. [22] The conditions are very mild and produce very good yields of the pentafluoroester. NHS has also been frequently been used to form the activated ester. These methods allow for the introduction of halogenated maleimides as well as the thiophenol derivatives.

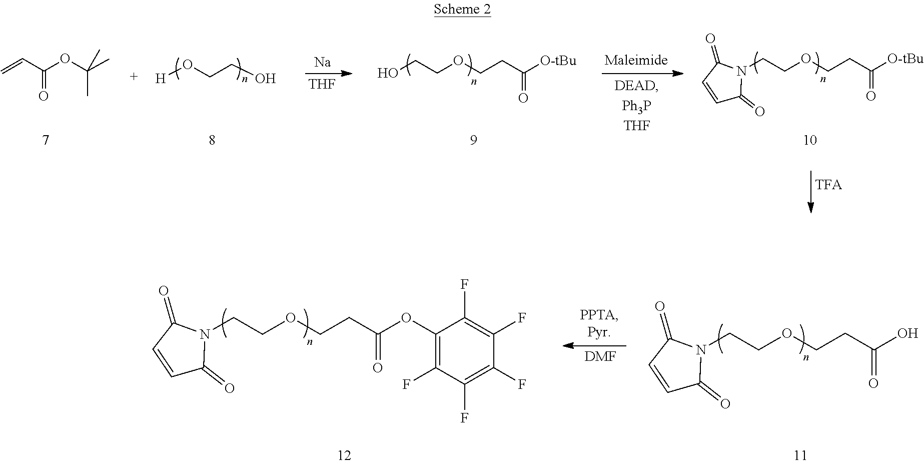

Scheme 3 shows the synthesis of a non-cleavable linker using the maleiminde as the key group to attach to a cysteine residue. With the incorporation of halogens on the maleimide, multiple cysteine residues or sulfides resulting from the cleavage of a disulfide bond can react at both halogenated positions. The synthesis of this versatile linker begins with the condensation of maleic anhydride (with or without halogens) with 3-(2-(2-(2-aminoethoxy) ethoxy)ethoxy)propanoic acid in refluxing acid to give a near quantitative yield of compound 15. [23] Carboxylic acid 15 can be converted to the pentafluoro ester through either a DCC coupling in ethyl acetate or using perfluorophenyl 2,2,2-trifluoroacetate (PPTA) in the presence of pyridine in DMF to give compound 16 [22]. The pentafluoro ester 16 is very amenable to displacement by amines under mild conditions to give a non-cleavable drug conjugate, where the maleimide moiety reacts under mild conditions with available cysteine residues and n is 1, 2, 3, 4, 5, 6, 7, 8, 9, or 10.

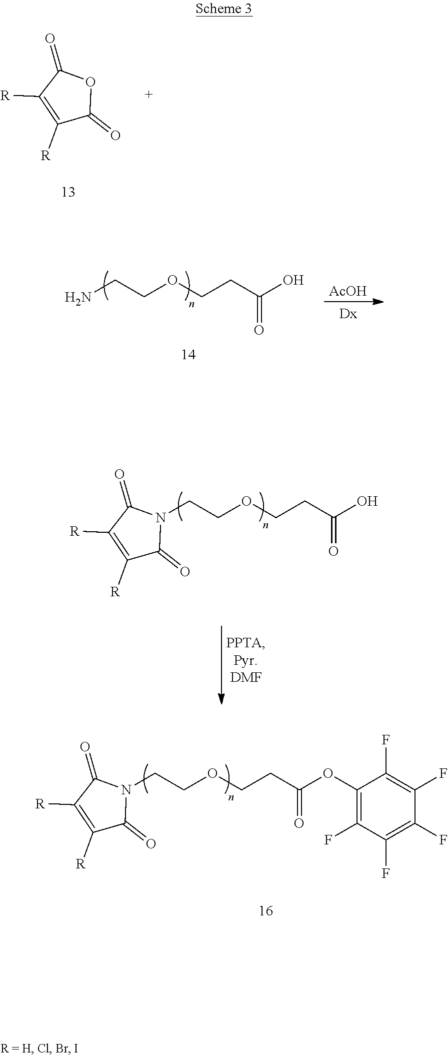

### Key Structures and Abbreviations

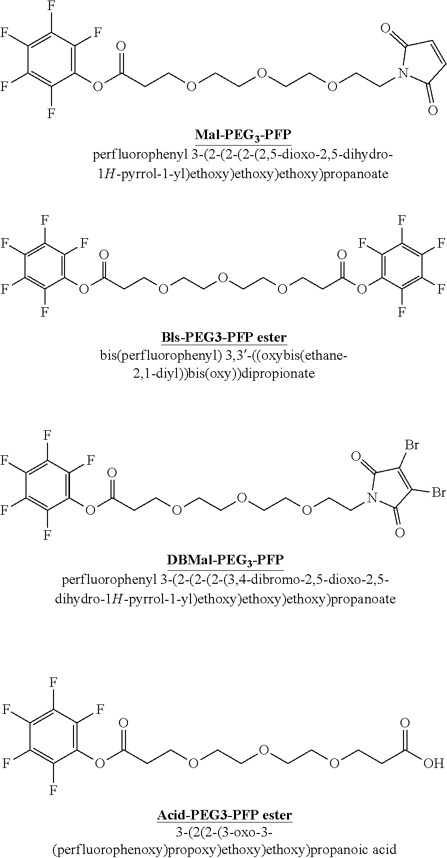 [SMILES: O=C(CCOCCOCCOCCN1C(=O)C=CC1=O)Oc1c(F)c(F)c(F)c(F)c1F ; O=C(CCOCCOCCOCCC(=O)Oc1c(F)c(F)c(F)c(F)c1F)Oc1c(F)c(F)c(F)c(F)c1F ; O=C(CCOCCOCCOCCN1C(=O)C(Br)=C(Br)C1=O)Oc1c(F)c(F)c(F)c(F)c1F ; O=C(O)CCOCCOCCOCCC(=O)Oc1c(F)c(F)c(F)c(F)c1F]

To improve the stability of the heterobifunctional cross-linking reagent, 6-maleimidohexanoic acid pentafluorophenyl ester (Mal-PEGn-PFP, where n is defined below) may be prepared as a substitute for the more popular 6-maleimidohexanoic acid N-hydroxysuccinimide ester (MHSu).[24] Mal-PEGn-PFP is prepared in two steps in very good yields to give compounds 12 and 16 as shown herein in Schemes 2 and 3. [14, 15] Subsequent reaction of compound 6 with Mal-PEG₃-PFP and DBMal-PEG₃-PFP in DMF provided very good yields of compounds 17 and 18 shown in Scheme 4.

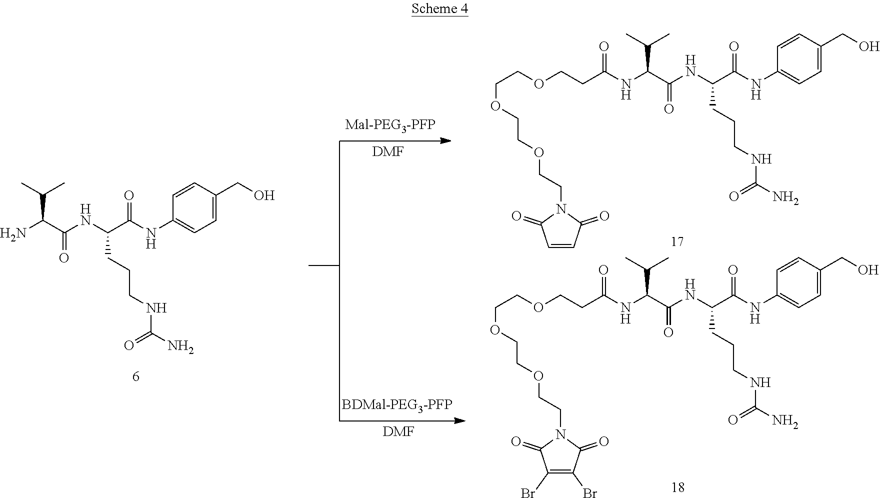

As shown in Scheme 5, compound 17 can be activated using at least two different methods to facilitate the attachment of various therapeutic compound as payloads before bio-conjugation to either Cys or Lys residues of biologically active polypeptides, such as bispecific antibodies and soluble checkpoint FC's. Initially, Bis(4-nitrophenyl) carbonate was reacted with compound 7 in the presence of N,N-Diisopropylethylamine and DMF to give carbonate 12. [24] Alternatively, N,N-disuccinimidyl carbonate can be used in a similar fashion under the same conditions. Both the p-Nitrophenol and the N-Hydroxysuccinimde function as leaving groups in the subsequent reaction. In a similar fashion, the mesylate 20 can be generated from the benzyl alcohol using Ms₂O in dichloromethane.

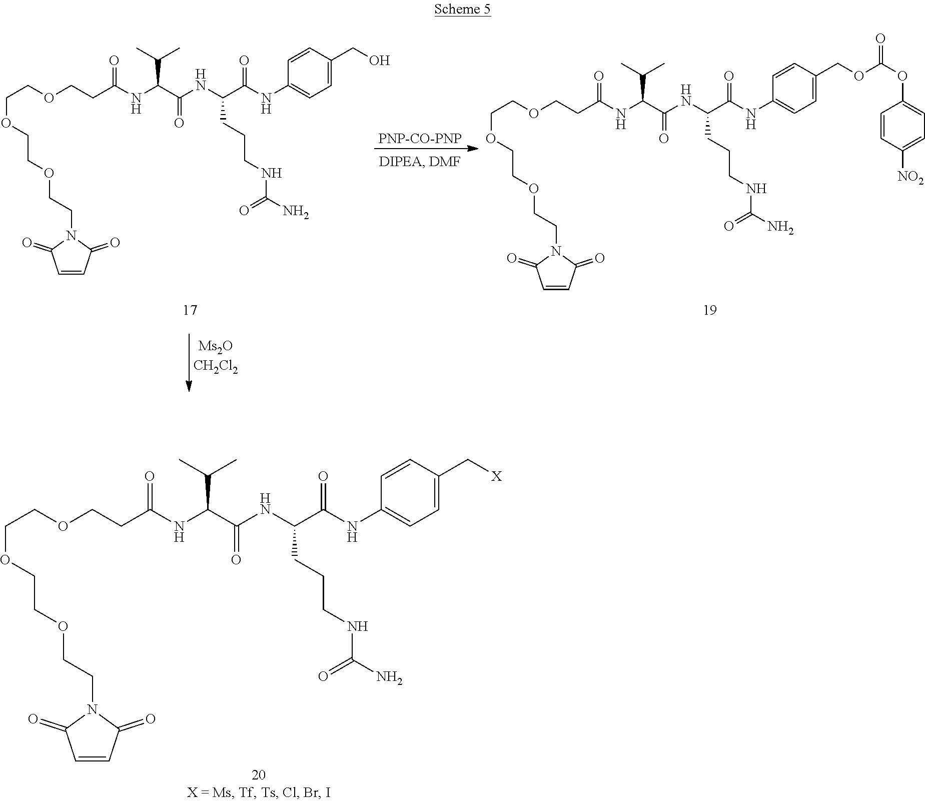

Nitro-carbonate 19 can react with the therapeutic compound to form carbonyl compound 21 which can take the form of a carbamate or a carbonate, depending on whether the reactive site on the therapeutic compound is an amine or an alcohol. When the therapeutic compound possesses a tertiary or heteroaryl amine, it can be reacted with compound 20 to generate a quaternary amine attachment to deliver the payload. [25]

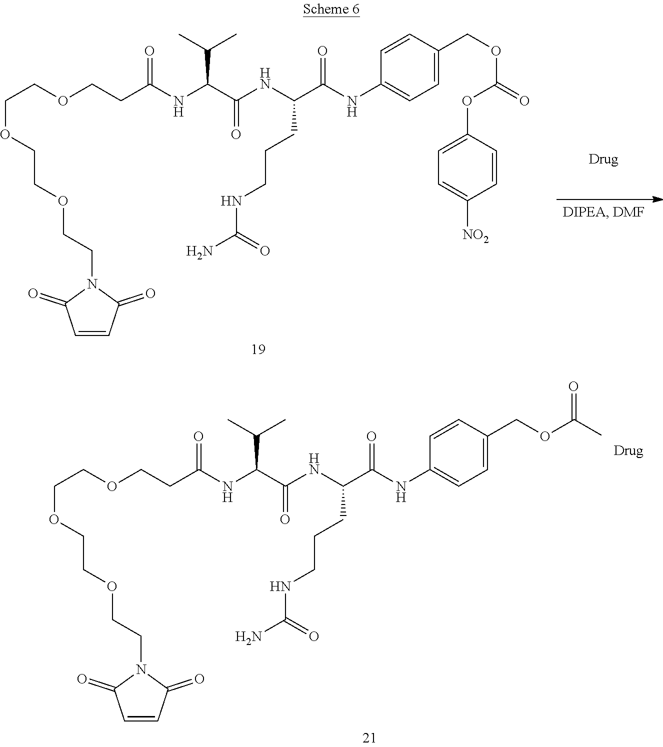 [SMILES: CC(C)[C@H](NC(=O)CCOCCOCCOCCN1C(=O)C=CC1=O)C(=O)N[C@@H](CCCNC(N)=O)C(=O)Nc1ccc(COC(=O)Oc2ccc([N+](=O)[O-])cc2)cc1 ; CC(=O)OCc1ccc(NC(=O)[C@H](CCCNC(N)=O)NC(=O)[C@@H](NC(=O)CCOCCOCCOCCN2C(=O)C=CC2=O)C(C)C)cc1]

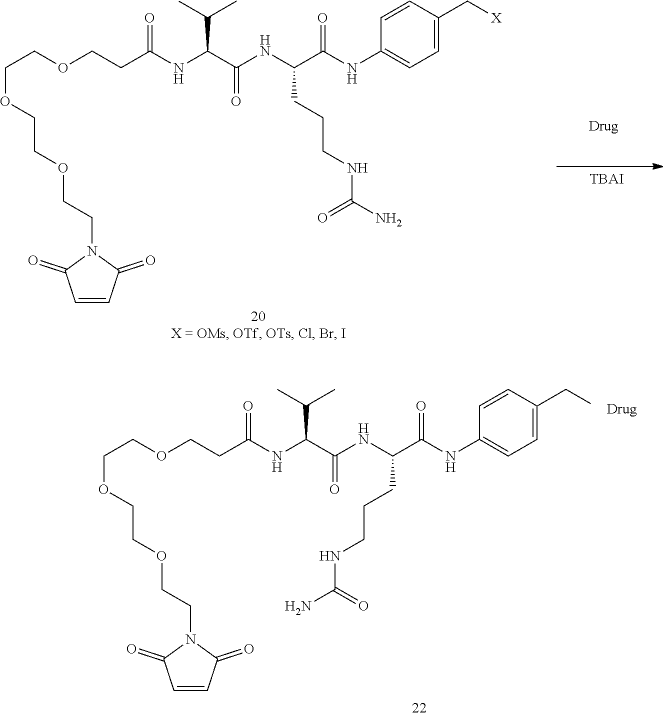

Below in Scheme 7, benzyl alcohol 5 was converted to a Mesylate (or similar leaving group) and subsequently replaced by the drug to form a quaternary ammonium bond with the linker. The mesylate (and potentially the triflate) offer an improvement in reactivity over the Iodo or Bromo substituted benzyl group. To finish functionalizing the PDC, the Fmoc group was deprotected and attach to a linker capable of reacting with Lysine residues on the protein as shown in Scheme 8.

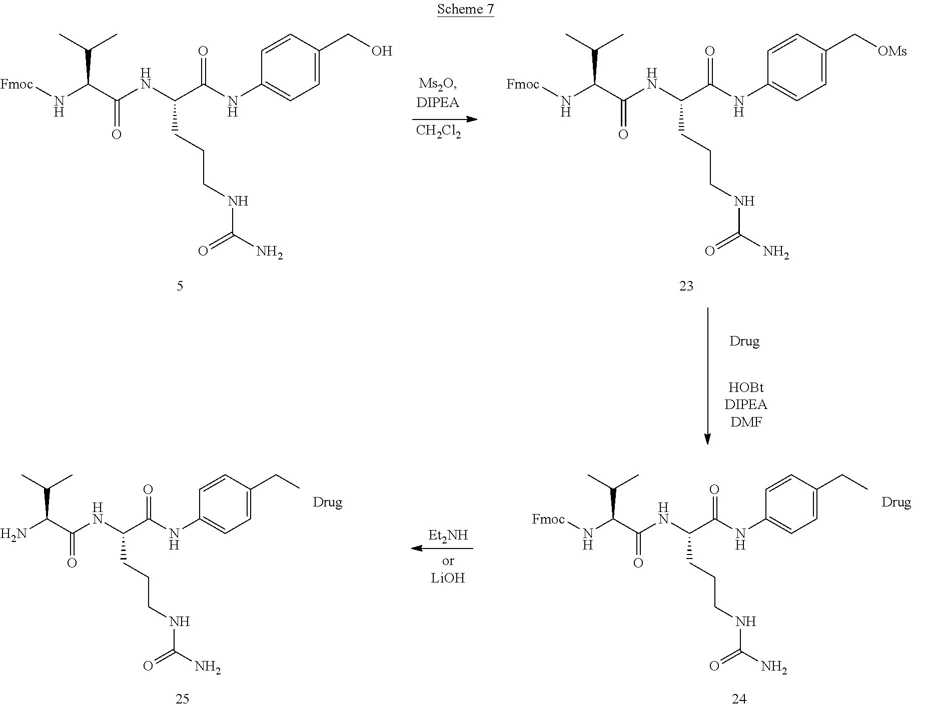

Scheme 8 shows the final deprotection of the Fmoc group of Valine, making the primary amine available for reaction with the Bis-PEGS-PFP ester to generate compound 27. The alternative to this one step conversion is reacting compound 25 with Acid-PEG3-PFP-Ester to generate the carboxylic acid 26. The carboxylic acid can then be converted to the PFP ester using PPTA in DMF with pyridine as a solvent.

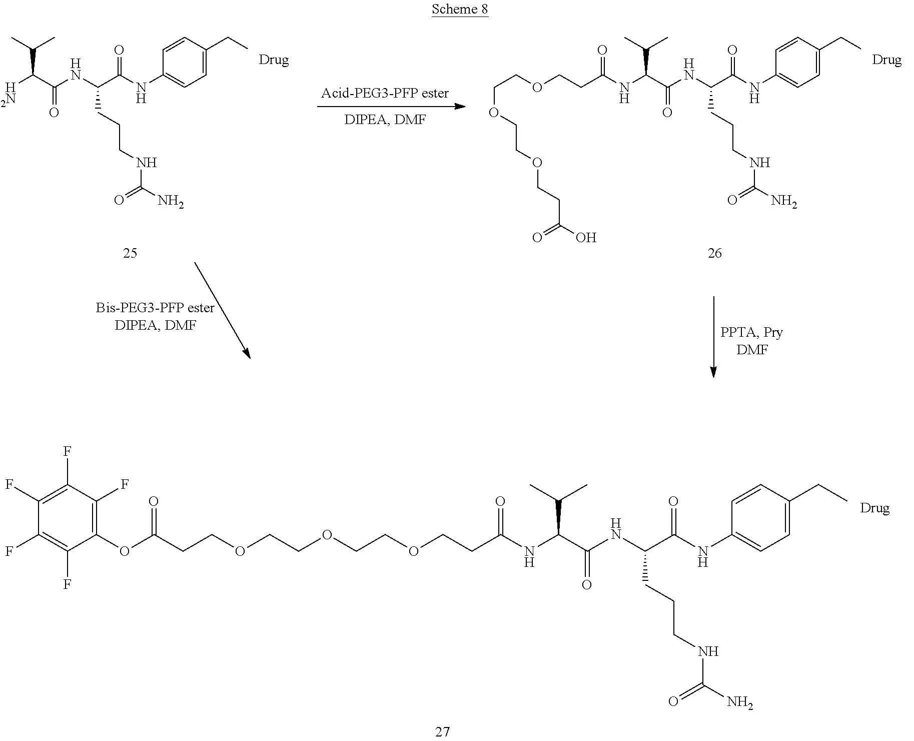 [SMILES: CCc1ccc(NC(=O)[C@H](CCCNC(N)=O)NC(=O)[C@@H](N)C(C)C)cc1 ; CCc1ccc(NC(=O)[C@H](CCCNC(N)=O)NC(=O)[C@@H](NC(=O)CCOCCOCCOCCC(=O)O)C(C)C)cc1 ; CCc1ccc(NC(=O)[C@H](CCCNC(N)=O)NC(=O)[C@@H](NC(=O)CCOCCOCCOCCC(=O)Oc2c(F)c(F)c(F)c(F)c2F)C(C)C)cc1]

### Glucuronide Based Linkers

The Glucuronide scaffold has been utilized in assisting the transport of active biological agents and therapeutic compound payloads to a specific location.[26, 27, 28, 29, 30] As shown in Scheme 9, the synthesis of the transport molecule may be initiated with the commercially available (2S,3R,4S,5S,6S)-6-(methoxycarbonyl)tetrahydro-2H-pyran-2,3,4,5-tetrayl tetraacetate) undergoing a mild bromide substitution of the acetate group through the reaction of compound 28 with HBr in acetic acid to form compound 29. The bromide may be replaced by reacting 4-hydroxy-3-nitrobenzaldehyde with silver oxide in acetonitrile resulting in the formation of compound 30. Reduction of the formal group with sodium borohydride in methanol to give compound 31, may be followed by the reduction of the nitro group by palladium hydroxide to give the amino alcohol 32.

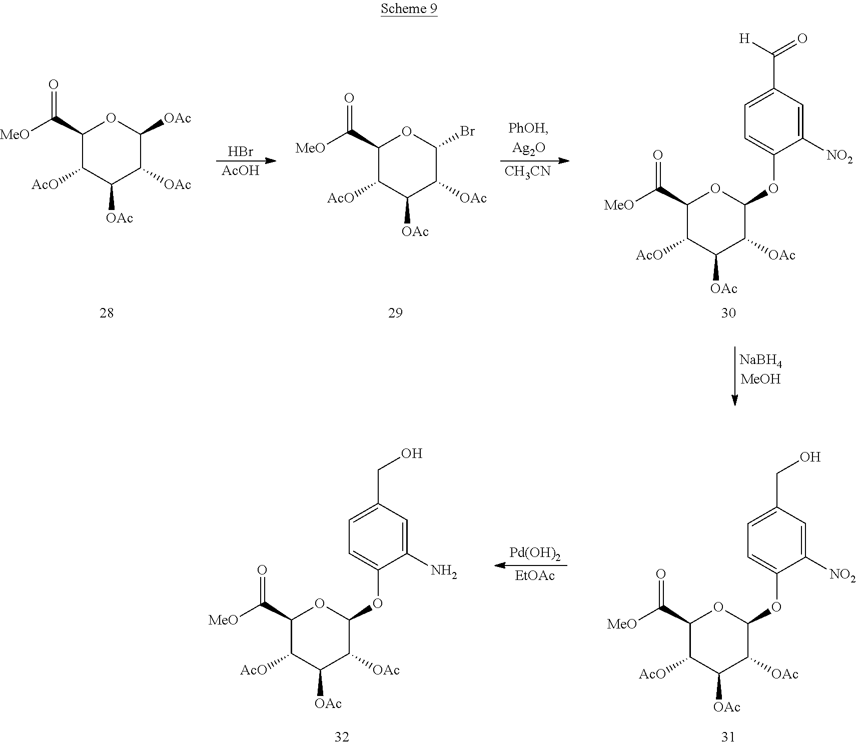

Commercially available Fmoc-B-Alanine was sonicated with thionyl chloride in dichloromethane at room temperature to form the resulting (9H-fluoren-9-yl) methyl (3-chloro-3-oxopropyl)carbamate or compound 34.

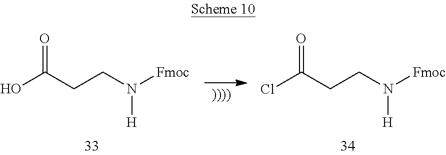

As shown in Scheme 11, the amino group of compound 32 can be selectively reacted with (9H-fluoren-9-yl) methyl (3-chloro-3-oxopropyl)carbamate followed by the formation of mesylate intermediate 35. Mesylate 35 can be reacted with the tertiary amine of the therapeutic compound to form a stable bio-reversible quaternary ammonium linkage of compound 36. Deprotection of the acetate groups can be performed with LiOH, which also lead to the removal of the Fmoc group to give compound 37.

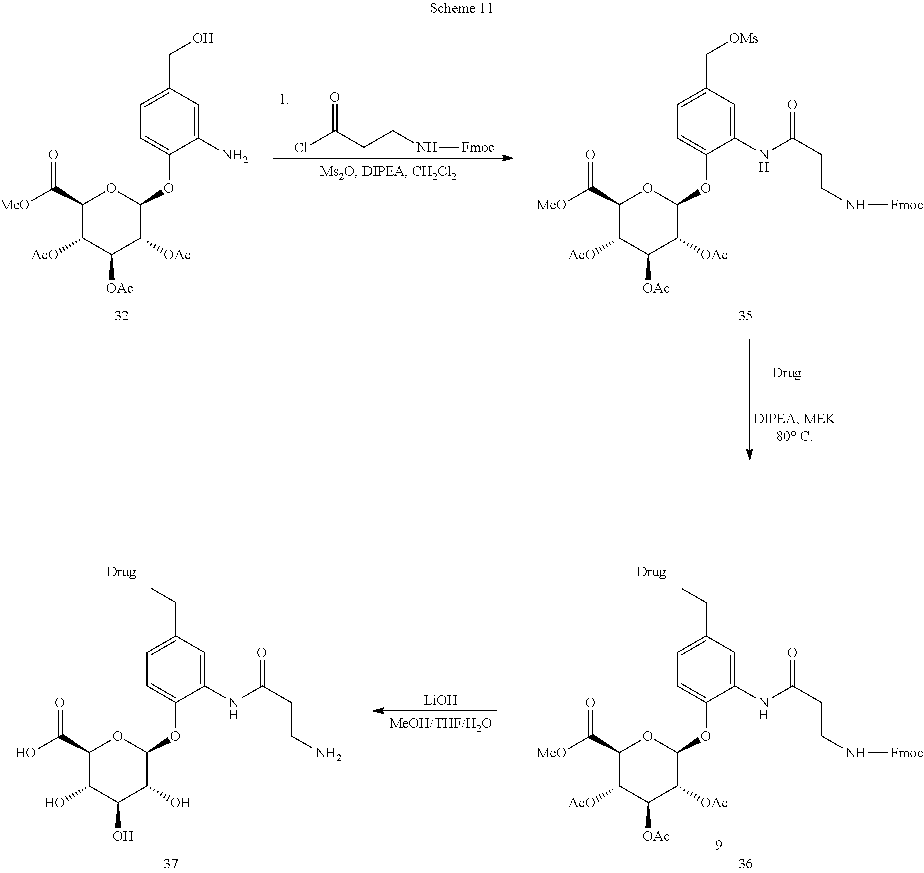

As shown in Schemes 12 and 13, compound 37 can be functionalized to selectively react with either the Cys or Lys residues on the biologically active polypeptides or monoclonal antibody. For a bioconjugation to the Cys residue, the free amine on compound 37 can be reacted with the heterobifunctional cross-linking reagent Mal-PEG3-PFP allowing 38 to react with a single Cysteine of the protein; or reacted with DBMal-PEG3-PFP to form compound 39. Compound 39 can be utilized to react with reduced disulfides and form a re-bridged disulfide to maintain the structural integrity of the protein. For the bioconjugation to Lys residues of biologically active polypeptides or monoclonal antibodies, the free amine 37 is first reacted with Acid-PEG3-PFP ester to give the acid 40, followed by the conversion to the PFP ester 41 using PPTA with pyridine in a solution of DMF. Alternatively, the PFP ester 41 can be formed directly from compound 36 through a reaction with Bis-PEG3-PFP ester shown in Scheme 7.

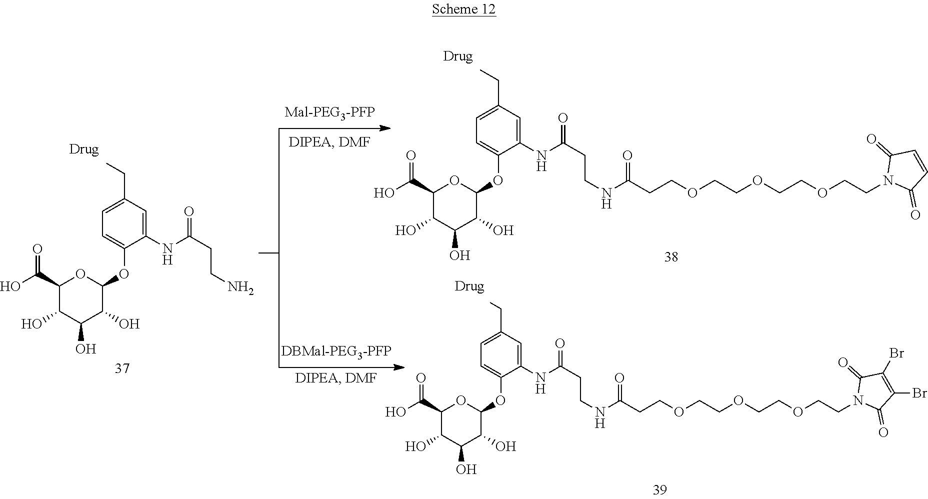 [SMILES: CCc1ccc(O[C@@H]2O[C@H](C(=O)O)[C@@H](O)[C@H](O)[C@H]2O)c(NC(=O)CCN)c1 ; "DIPEA, DMF" ; "DIPEA, DMF" ; CCc1ccc(O[C@@H]2O[C@H](C(=O)O)[C@@H](O)[C@H](O)[C@H]2O)c(NC(=O)CCNC(=O)CCOCCOCCOCCN2C(=O)C=CC2=O)c1 ; CCc1ccc(O[C@@H]2O[C@H](C(=O)O)[C@@H](O)[C@H](O)[C@H]2O)c(NC(=O)CCNC(=O)CCOCCOCCOCCN2C(=O)C(Br)=C(Br)C2=O)c1]

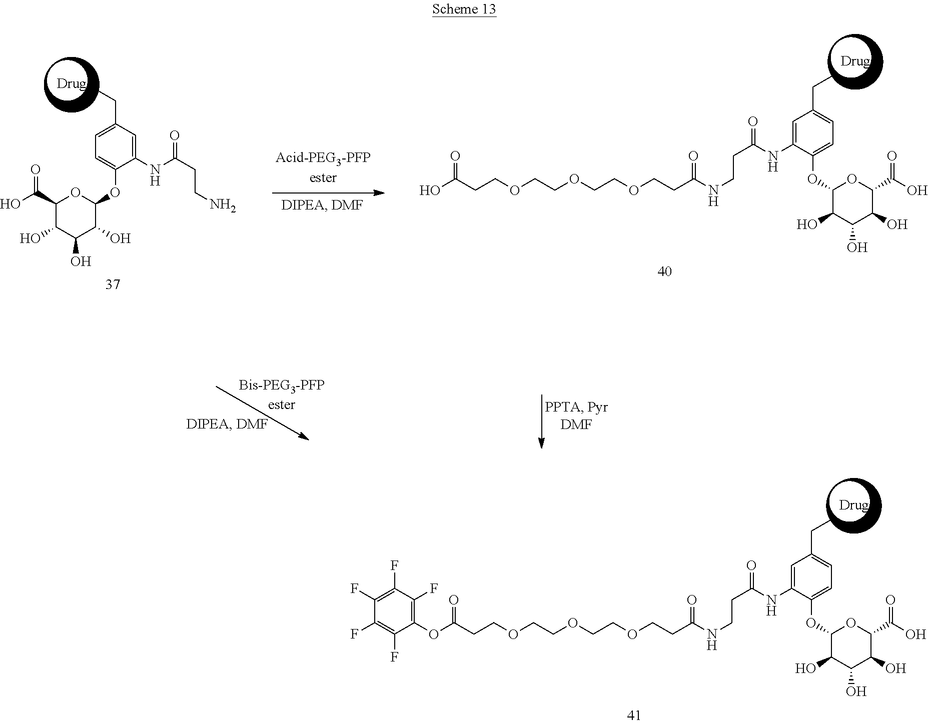 [SMILES: CCc1ccc(O[C@@H]2O[C@H](C(=O)O)[C@@H](O)[C@H](O)[C@H]2O)c(NC(=O)CCN)c1 ; CCc1ccc(O[C@@H]2O[C@H](C(=O)O)[C@@H](O)[C@H](O)[C@H]2O)c(NC(=O)CCNC(=O)CCOCCOCCOCCC(=O)O)c1 ; CCc1ccc(O[C@@H]2O[C@H](C(=O)O)[C@@H](O)[C@H](O)[C@H]2O)c(NC(=O)CCNC(=O)CCOCCOCCOCCC(=O)Oc2c(F)c(F)c(F)c(F)c2F)c1]

### Pyridazinedione Chemistry

Dihalomaleimide compounds often encounter stability problems and hydrolyze to form the open amide-carboxylic acid[x]. The pyridazinediones were developed to address these challenges, as they are resistant to hydrolysis, contain the same two carbon bridge as the maleimides and they possess two sites available for dual functionalization. The synthesis of the pyridazinedione begins with the alkylation of the Boc protected hydrazine to give the monoalkylated derivative of the hydrazine. The monoalkylated intermediate can be reacted with t-butylacrylate in t-BuOH to give intermediate 43. Compound 43 was reacted with dibromomaleic acid in acetic acid to give compound 44.

The protected amino PEG ester was coupled to compound 44 using typical amu\ide forming chemistry to produce compound 45. Subsequent deprotection of t-butyl ester and attachment of the pentafluoro phenol result in compound 46. Scheme 15 shows the reaction of the activated pyridazinedione PEG ester with the glucuronide linked drug intermediate 37 to give compound 47, which will be reacted with cysteine residues for the bioconjugation reaction.

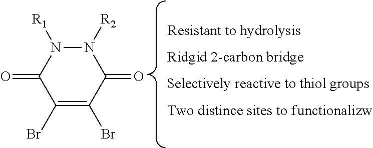

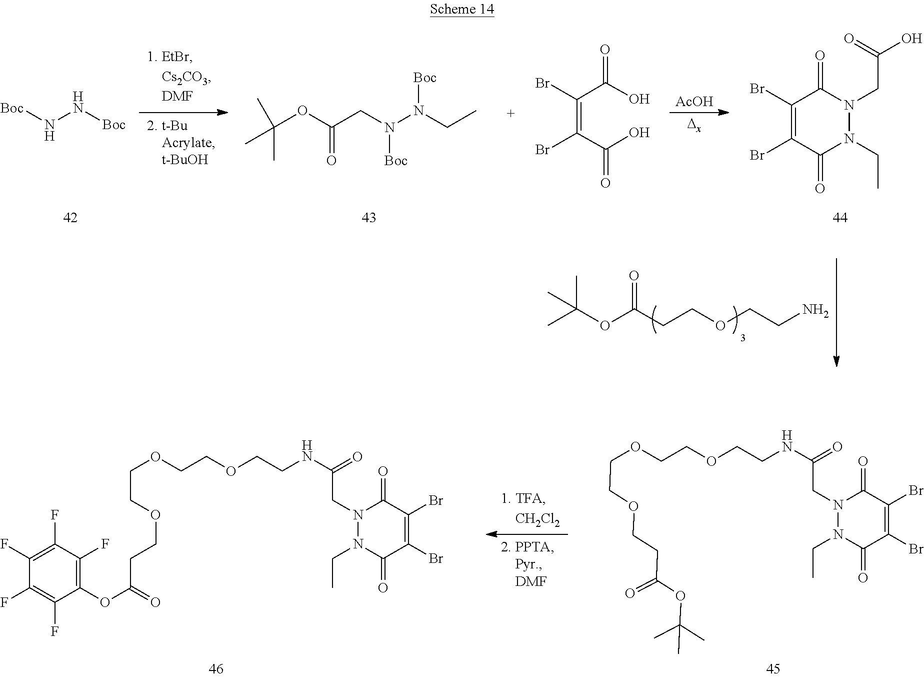

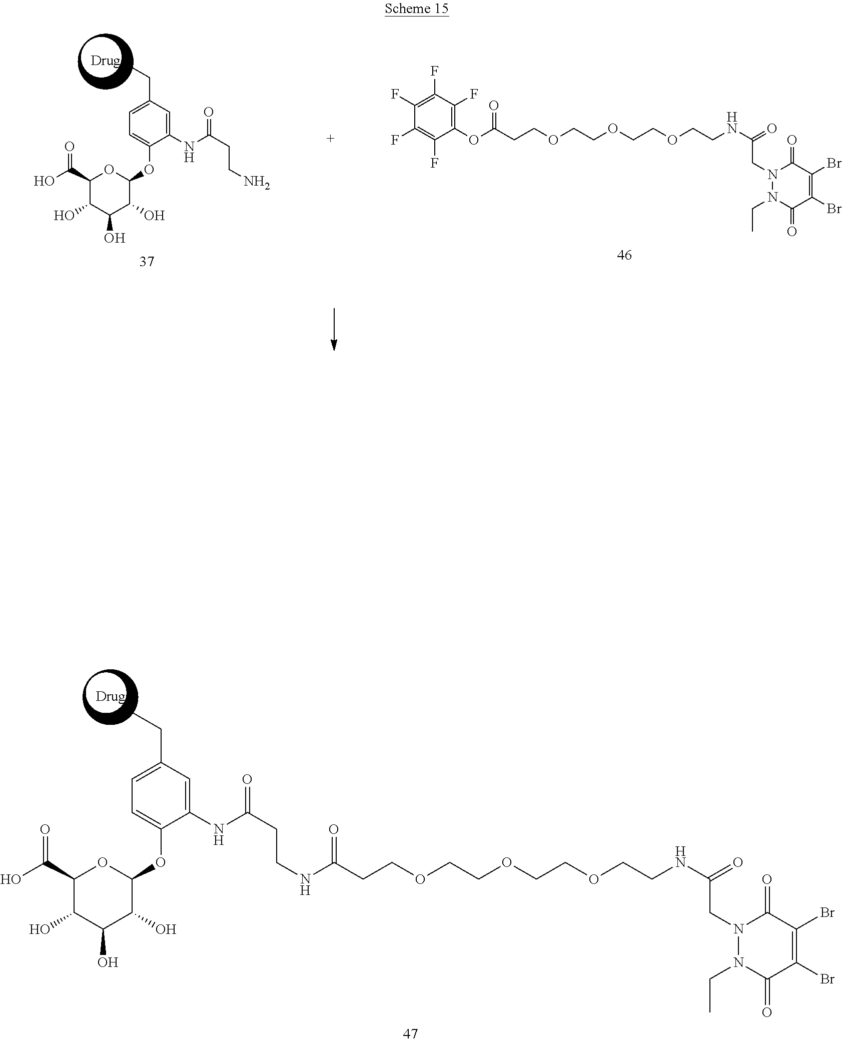

### Additional Linkers

Attachment of the payload to the drug conjugate often requires a functional linker with very good solubility properties as well as serum stability. Glycols may be used in this capacity. Below in schemes 16, 17, and 18, there are several approaches for the synthesis of glycol side chains that allow for easy attachment of both the payload as well as the conjugate linkers. In Scheme 16, an ethylene glycol may be reacted with Tosyl chloride to produce the Bis-Tosylate. The Bis-Tosylate may then be reacted with the Bis-tert-butyl carbamate to form compound 50. Subsequent reaction with LiBr results in the bromide substitution of the Tosylate to give compound 51.[31] In Schemes 16, 17, and 18, n is 1, 2, 3, 4, 5, 6, 7, 8, 9, or 10.

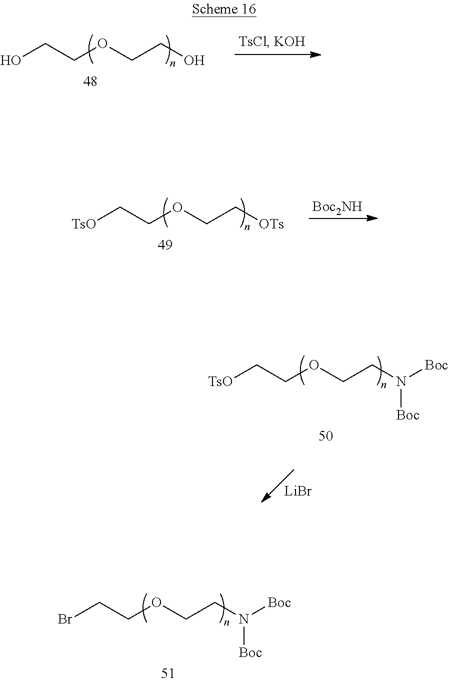

Glycol 52 in Scheme 15 has a terminal chloride, which is available for substitution. Sodium azide may be used to displace the chloride atom and the azide was then reduced with triphenylphosphine followed by protection of the amine with Boc anhydride to give compound 54. The hydroxyl group may then finally be converted to a bromide using triphenylphosphine and carbon tetrabromide to give compound 55.[32]

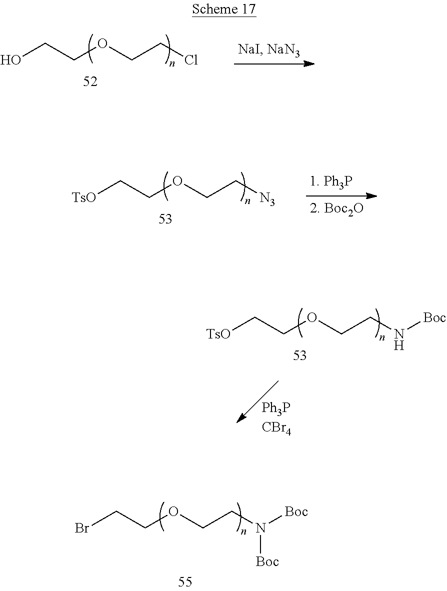

In Scheme 18, the di-iodinated glycol is reacted with the Bis-Boc-carbamate to produce the Bis-Boc protected amino-iodo-glycol 57.

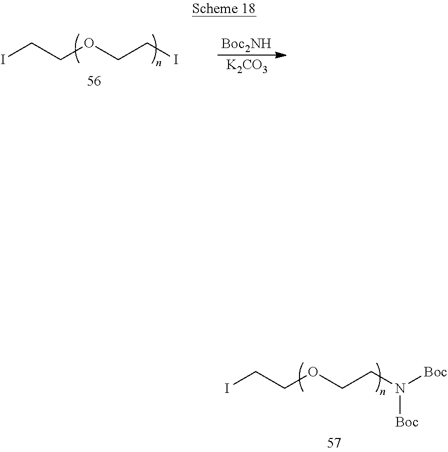

### SEQUENCE LISTING

Below is a sequence listing of the nucleotide and/or amino acid sequences and associated information. This sequence listing is also being submitted as ASCII text file. The ASCII Text file is named OncologyST25.txt and was created on Jun. 20 2021 and has a file size of 34 KB and is hereby incorporated by reference.

What is claimed is:

1. A compound having a structure of Formula (I):
X₂ₐ—(X₃)ₘ (I),
and pharmaceutically acceptable salts, solvates, hydrates, isomers, or tautomers thereof, wherein:
m is 1, 2, 3, 4, 5, 6, 7, 8, 9, or 10;
X₂ₐ is a linker; and
X₃ is a therapeutic compound.

2. A compounds having the structure of Formula (II):
X₁—[X₂—(X₃)ₘ]ₙ (II),
and pharmaceutically acceptable salts, solvates, hydrates, isomers, or tautomers thereof, wherein:
m is 1, 2, 3, 4, 5, 6, 7, 8, 9, or 10;
n is 1, 2, 3, 4, 5, 6, 7, 8, 9, 10, or 11;
X₁ is a biologically active polypeptide or hormone;
X₂ is a linker; and
X₃ is a therapeutic compound.

3. A compound having the structure of Formula (III):
X₁—[X₂—(X₃)ₘ]ₙ (III),
and pharmaceutically acceptable salts, solvates, hydrates, isomers, or tautomers thereof, wherein:
m is 1, 2, 3, 4, 5, 6, 7, 8, 9, or 10;
n is 1, 2, 3, 4, 5, 6, 7, 8, 9, 10, or 11;
X₁ is bispecific antibodies and soluble checkpoint FC's thereof;
X₂ is a linker; and
X₃ is a therapeutic compound.


SEQUENCE LISTING (9 sequences)

SEQ ID NO: 1 - protein, 375 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGSAMPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 442 aa, Homo sapiens
QSALTQPRSVSGSPGQSVTISCTGTSSDVGGYNYVSWYQQHPGKAPKLMIYDVSKRPSGVPDRFSGSKSGNTASLTISGLQAEDEADYYCSSYADSVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECSGSAMPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 355 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRGSAMPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 354 aa, Homo sapiens
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDGSAMPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 365 aa, Homo sapiens
QRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPGSAMPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 514 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIGGGGSGGGGSGGGGSGGGGSQVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKAPGDYTAYFDYWGQGTLVTVSSGGGGSELVLTQSPATLSLSPGERATLSCRASQSVSSYLAWYQQKPGQAPRLLIYGASSRATGIPDRFSGSGSGTDFTLTISRLEPEDFAVYYCQQYGSSPPYTFGQGTKVEIK

SEQ ID NO: 7 - protein, 521 aa, Homo sapiens
EVQLVESGGGLVQPGGSLRLSCAASGFTFSDSWTHWVRQAPGKGLEWVAWLSPYGGSTYYDSVKGRFTISDTSKNTYLQMNSLREDTVYYCRRHWPGGFDYWGQGTLVTVSSSTKGPSVFPLAPSSKSTSGGGGGGSDLQMTQSPSSLSASVGDRVTLTCRASQDVSTAVAWYQQKPGKAPKLYSASFLYSGVPSRFSGSGSGTDFTLTLSSLQPEDFATYYCQQYLYHPATFGQGTKVELKRTVAAPSVFLGGGGSGGGGSGGGGSGGGGSQVQLVQSGGGLVQPGGSLRLSCAASGFTFSSYAMSWVRQAPGKGLEWVSALSGSGGSTYYADSVKGRFTLSRNSKNTLYLQMNSLRAEDTAVYYCAKAPGDYTAYFDYWGQGTLVTVSSGGGGSELVLTQSPATTSTSPGERATFSCRASQSVSSYTAWYQQKPGQAPRTTYGASSRATGTPDRFSGSGSGTDFTLTTSRLEPEDFAVYYCQQYGSSPPYTFGQGTKVETKSGGGGSYSMEHFRWGKPV

SEQ ID NO: 8 - protein, 521 aa, Homo sapiens; identical to SEQ ID NO: 7

SEQ ID NO: 9 - protein, 495 aa, Homo sapiens
QVQLVQSGGGLVQPGGSLRLSCAASGFTFSSYAMSWVRQAPGKGLEWVSALSGSGGSTYYADSVKGRFTLSRDNSKNTLYLQMNSLRAEDTAVYYCAKAPGDYTAYFDYWGQGTLVTVSSGGGGSGGGGSGGGGSGGGGSEIVLTQSPATSILSPGERATSTCRASQSVSSYIAWYQQKPGQAPRIYGASSRATGTPDRFSGSGSGTDFTLTLSRLEPEDFAVYYCQQYGSSPPYTFGQGTKVELKEPKSCDKTHTCPPCPAPELLGGPSVFLFPPKPKDTLMLSRTPEVTCVVVDVSHEDPEVKFNWYVDGVEVHNAKTKPREEQYNSTYRVVSVTTVTTQDWTNGKEYKCKVSNKAPAPTEKTTSKAKGQPREPRVYTTPPSRDETTKNQWSTTCTVKGFYPSDTAVFWESNGQPENNYKTTPPVLVSDGSFTLYSKLTVDKSRWQQGNWFSCSVMHEALHNHYTQKSLSLSPGKSGGGGSYSMEHFRWGKPV